(19) United States
(12) Patent Application Publication
(10) Pub. No.: US 2019/0002563 A1
(43) Pub. Date: Jan. 3, 2019

(54) Bispecific Monovalent Diabodies That are Capable of Binding B7-H3 and CD3, and Uses Thereof

(71) Applicant: MacroGenics, Inc., Rockville, MD (US)

(72) Inventors: Leslie S. Johnson, Darnestown, MD (US); Paul A. Moore, North Potomac, MD (US); Ezio Bonvini, Potomac, MD (US); Ling Huang, Bethesda, MD (US); Kalpana Shah, Boyds, MD (US); Ralph Alderson, North Potomac, MD (US); Gurunadh Reddy Chichili, Germantown, MD (US)

(73) Assignee: MacroGenics, Inc., Rockville, MD (US)

(21) Appl. No.: 15/752,367

(22) PCT Filed: Aug. 12, 2016
(86) PCT No.: PCT/US2016/046680

§ 371 Date: Feb. 13, 2018

Related U.S. Application Data
(60) Provisional application No. 62/206,051, filed on Aug. 17, 2015.
(60) Provisional application No. 62/280,318, filed on Jan. 19, 2016.

(51) Int. Cl. C07K 16/28; A61P 35/00

(52) U.S. Cl. CPC C07K 16/2809; C07K 16/2827; A61P 35/00; A61K 2039/505; C07K 2317/626; C07K 2317/92; C07K 2317/33; C07K 2317/31

(57) ABSTRACT

The present invention is directed to B7-H3×CD3 bispecific monovalent diabodies, and particularly, to B7-H3×CD3 bispecific monovalent Fc diabodies, that are capable of simultaneous binding to B7-H3 and CD3. The invention is also directed to pharmaceutical compositions that contain such bispecific monovalent Fc diabodies. The invention is additionally directed to methods for the use of such diabodies in the treatment of cancer and other diseases and conditions.

### CROSS-REFERENCE TO RELATED APPLICATIONS

This application claims priority to U.S. Patent Application Ser. No. 62/206,051 (filed on Aug. 17, 2015; pending) and 62/280,318 (filed on Jan. 19, 2016), each of which applications is herein incorporated by reference in its entirety.

### REFERENCE TO SEQUENCE LISTING

This application includes one or more Sequence Listings pursuant to 37 C.F.R. 1.821 et seq., which are disclosed in computer-readable media (file name: 1301-0123PCT_ST25.txt, created on Aug. 10, 2016, and having a size of 70,481 bytes), which file is herein incorporated by reference in its entirety.

### FIELD OF THE INVENTION

The present invention is directed to bispecific monovalent diabodies that possess one binding site specific for an epitope of B7-H3 and one binding site specific for an epitope of CD3 (i.e., a “B7-H3×CD3 bispecific monovalent diabody”). Most preferably, such B7-H3×CD3 bispecific monovalent diabodies are composed of three polypeptide chains and possess one binding site specific for an epitope of B7-H3 and one binding site specific for an epitope of CD3 and additionally comprise an immunoglobulin Fc Domain (i.e., a “B7-H3×CD3 bispecific monovalent Fc diabody”). The bispecific monovalent Fc diabodies of the present invention are capable of simultaneous binding to B7-H3 and CD3. The invention is directed to pharmaceutical compositions that contain such bispecific monovalent Fc diabodies. The invention is additionally directed to methods for the use of such diabodies in the treatment of cancer and other diseases and conditions.

### BACKGROUND OF THE INVENTION

### I. The B7 Superfamily and B7-H3

The growth and metastasis of tumors depends to a large extent on their capacity to evade host immune surveillance and overcome host defenses. Most tumors express antigens that can be recognized to a variable extent by the host immune system, but in many cases, an inadequate immune response is elicited because of the ineffective activation of effector T cells (Khawli, L. A. et al. (2008) “Cytokine, Chemokine, and Co-Stimulatory Fusion Proteins for the Immunotherapy of Solid Tumors,” Exper. Pharmacol. 181:291-328).

B7-H3 is a member of the B7 superfamily of immunoglobulin molecules. Members of the B7 superfamily possess an immunoglobulin-V-like domain and an immunoglobulin-C-like domain (e.g., IgV and IgC, respectively) (Sharpe, A. H. et al. (2002) “The B7-CD28 Superfamily,” Nature Rev. Immunol. 2:116-126). The IgV and IgC domains of B7-superfamily members are each encoded by single exons, with additional exons encoding leader sequences, transmembrane and cytoplasmic domains. The cytoplasmic domains are short, ranging in length from 19 to 62 amino-acid residues and can be encoded by multiple exons (Collins, M. et al. (2005) “The B7 Family Of Immune Regulatory Ligands,” Genome Biol. 6:223.1-223.7). Members of the B7 superfamily are predicted to form back-to-back, non-covalent homodimers at the cell surface, and such dimers have been found with respect to B7-1 (CD80) and B7-2 (CD86). B7-1 (CD80) and B7-2 (CD86) exhibit have dual specificity for the stimulatory CD28 receptor and the inhibitory CTLA-4 (CD152) receptor (Sharpe, A. H. et al. (2002) “The B7-CD28 Superfamily,” Nature Rev. Immunol. 2:116-126).

B7-H3 (CD276) is unique in that the major human form contains two extracellular tandem IgV-IgC domains (i.e., IgV-IgC-IgV-IgC) (Collins, M. et al. (2005) “The B7 Family Of Immune-Regulatory Ligands,” Genome Biol. 6:223.1-223.7). Although initially thought to comprise only 2 Ig domains (IgV-IgC) (Chapoval, A. et al. (2001) “B7-H3: A Costimulatory Molecule For T Cell Activation and IFN-γ Production,” Nature Immunol. 2:269-274; Sun, M. et al. (2002) “Characterization of Mouse and Human B7-H3 Genes,” J. Immunol. 168:6294-6297) the four immunoglobulin extracellular domain variant (“4Ig-B7-H3”) has been found to be the more common human form of the protein (Sharpe, A. H. et al. (2002) “The B7-CD28 Superfamily,” Nature Rev. Immunol. 2:116-126). However, the natural murine form is 2Ig, and it and the human 4Ig form exhibit similar function (Hofmeyer, K. et al. (2008) “The Contrasting Role Of B7-H3,” Proc. Natl. Acad. Sci. (U.S.A.) 105(30):10277-10278). The 4Ig-B7-H3 molecule inhibits the natural killer cell-mediated lysis of cancer cells (Castriconi, R. et al. (2004) “Identification Of 4Ig-B7-H3 As A Neuroblastoma Associated Molecule That Exerts A Protective Role From An NK Cell-Mediated Lysis,” Proc. Natl. Acad. Sci. (U.S.A.) 101(34):12640-12645). The 2Ig form of human B7-H3 has been found to promote T cell activation and IFN-γ production by binding to a putative receptor on activated T cells (Chapoval, A. et al. (2001) “B7-H3: A Costimulatory Molecule For T Cell Activation and IFN-γ Production,” Nature Immunol. 2:269-274; Xu, H. et al. (2009) “MicroRNA miR-29 Modulates Expression of Immunoinhibitory Molecule B7-H3: Potential Implications for Immune Based Therapy of Human Solid Tumors,” Cancer Res. 69(15):5275-6281). B7-H4 and B7-H3 are both potent inhibitors of immune function when expressed on tumor cells (Flies, D. B. et al. (2007) “The New B7s: Playing a Pivotal Role in Tumor Immunity,” J. Immunother. 30(3):251-260).

The mode of action of B7-H3 is complex, as the protein mediates both T cell co-stimulation and co-inhibition (Hofmeyer, K. et al. (2008) “The Contrasting Role Of B7-H3,” Proc. Natl. Acad. Sci. (U.S.A.) 105(30):10277-10278; Martin-Orozco, N. et al. (2007) “Inhibitory Costimulation And Anti-Tumor Immunity,” Semin. Cancer Biol. 17(4):288-298; Subudhi, S. K. et al. (2005) “The Balance Of Immune Responses: Costimulation Verse Coinhibition,” J. Mol. Med. 83:193-202). B7-H3 binds to TREM-like transcript 2 (TLT-2) and co-stimulates T cell activation, but also binds to as yet unidentified receptor(s) to mediate co-inhibition of T cells. In addition, B7-H3, through interactions with unknown receptor(s) is an inhibitor for natural killer cells and osteoblastic cells (Hofmeyer, K. et al. (2008) “The Contrasting Role Of B7-H3,” Proc. Natl. Acad. Sci. (U.S.A.) 105(30):10277-10278). The inhibition may operate through interactions with members of the major signaling pathways through which T cell receptors (TCR) regulate gene transcription (e.g., NFTA, NF-κB, or AP-1 factors).

B7-H3 co-stimulates CD4+ and CD8+ T cell proliferation. B7-H3 also stimulates IFN-γ production and CD8+ lytic activity (Chapoval, A. et al. (2001) “B7-H3: A Costimulatory Molecule For T Cell Activation and IFN-γ Production,” Nature Immunol. 2:269-274; Sharpe, A. H. et al. (2002) “The B7-CD28 Superfamily,” Nature Rev. Immunol. 2:116-126). However, the protein also possibly acts through NFAT (Nuclear Factor For Activated T Cells), NF-κB (Nuclear Factor Kappa B), and AP-1 (Activator Protein-1) factors to inhibit T cell activation (Yi. K. H. et al. (2009) “Fine Tuning The Immune Response Through B7-H3 And B7-H4,” Immunol. Rev. 229:145-151). B7-H3 is also believed to inhibit Th1, Th2, or Th17 in vivo (Prasad, D. V. et al. (2004) “Murine B7-H3 Is A Negative Regulator Of T Cells,” J. Immunol. 173:2500-2506; Fukushima, A. et al. (2007) “B7-H3 Regulates The Development Of Experimental Allergic Conjunctivitis In Mice,” Immunol. Lett. 113:52-57; Yi. K. H. et al. (2009) “Fine Tuning The Immune Response Through B7-H3 And B7-H4,” Immunol. Rev. 229:145-151). Several independent studies have shown that human malignant tumor cells exhibit a marked increase in expression of B7-H3 protein and that this increased expression was associated with increased disease severity (Zang, X. et al. (2007) “The B7 Family And Cancer Therapy: Costimulation And Coinhibition,” Clin. Cancer Res. 13:5271-5279), suggesting that B7-H3 is exploited by tumors as an immune evasion pathway (Hofmeyer, K. et al. (2008) “The Contrasting Role Of B7-H3,” Proc. Natl. Acad. Sci. (U.S.A.) 105(30):10277-10278).

Molecules that block the ability of a B7 molecule to bind to a T cell receptor (e.g., CD28) inhibit the immune system and have been proposed as treatments for autoimmune disease (Linsley, P. S. et al. (2009) “The Clinical Utility Of Inhibiting CD28-Mediated Co-Stimulation,” Immunolog. Rev. 229:307-321). Neuroblastoma cells expressing 4Ig-B7-H3 treated with anti-4Ig-B7-H3 antibodies were more susceptible to NK cells. However, it is unclear whether this activity can be attributed to only antibodies against the 4Ig-B7-H3 form because all reported antibodies raised against the 4Ig-B7-H3 also bound the two Ig-like form of B7-H3 (Steinberger, P. et al. (2004) “Molecular Characterization of Human 4Ig-B7-H3, a Member of the B7 Family with Four Ig-Like Domains,” J. Immunol. 172(4): 2352-2359 and Castriconi et al. (2004) “Identification Of 4Ig-B7-H3 As A Neuroblastoma Associated Molecule That Exerts A Protective Role From An NK Cell-Mediated Lysis,” Proc. Natl. Acad. Sci. (U.S.A.) 101(34):12640-12645).

B7-H3 is not expressed on resting B or T cells, monocytes, or dendritic cells, but it is induced on dendritic cells by IFN-γ and on monocytes by GM-CSF (Sharpe, A. H. et al. (2002) “The B7-CD28 Superfamily,” Nature Rev. Immunol. 2:116-126). The receptor(s) that bind B7-H3 have not been fully characterized. Early work suggested one such receptor would need to be rapidly and transiently up-regulated on T cells after activation (Loke, P. et al. (2004) “Emerging Mechanisms Of Immune Regulation: The Extended B7 Family And Regulatory T Cells.” Arthritis Res. Ther. 6:208-214). Recently, the TREM-like transcript 2 (TLT-2, or TREML2) receptor (King, R. G. et al. (2006) “Trem-Like Transcript 2 Is Expressed On Cells Of The Myeloid/Granuloid And B Lymphoid Lineage And Is Up-Regulated In Response To Inflammation,” J. Immunol. 176:6012-6021; Klesney-Tait, J. et al. (2006) “The TREM Receptor Family And Signal Integration,” Nat. Immunol. 7:1266-1273; Yi. K. H. et al. (2009) “Fine Tuning The Immune Response Through B7-H3 And B7-H4,” Immunol. Rev. 229:145-151), which is expressed on myeloid cells has been shown to be capable of binding B7-H3, and of thereby co-stimulating the activation of CD8+ T cells in particular (Zang, X. et al. (2003) “B7x: A Widely Expressed B7 Family Member That Inhibits T Cell Activation,” Proc. Natl. Acad. Sci. (U.S.A.) 100:10388-10392; Hashiguchi, M. et al. (2008) “Triggering Receptor Expressed On Myeloid Cell-Like Transcript 2 (TLT-2) Is A Counter-Receptor For B7-H3 And Enhances T Cell Responses,” Proc. Natl. Acad. Sci. (U.S.A.) 105(30): 10495-10500; Hofmeyer, K. et al. (2008) “The Contrasting Role Of B7-H3,” Proc. Natl. Acad. Sci. (U.S.A.) 105(30): 10277-10278).

In addition to its expression on neuroblastoma cells, human B7-H3 is also known to be expressed on a variety of other cancer cells (e.g., gastric, ovarian and non-small cell lung cancers). B7-H3 protein expression has been immunohistologically detected in tumor cell lines (Chapoval, A. et al. (2001) “B7-H3: A Costimulatory Molecule For T Cell Activation and IFN-γ Production,” Nature Immunol. 2:269-274; Saatian, B. et al. (2004) “Expression Of Genes For B7-H3 And Other T Cell Ligands By Nasal Epithelial Cells During Differentiation And Activation,” Amer. J. Physiol. Lung Cell. Mol. Physiol. 287:L217-L225; Castriconi et al. (2004) “Identification Of 4Ig-B7-H3 As A Neuroblastoma Associated Molecule That Exerts A Protective Role From An NK Cell-Mediated Lysis,” Proc. Natl. Acad. Sci. (U.S.A.) 101(34):12640-12645); Sun, M. et al. (2002) “Characterization of Mouse and Human B7-H3 Genes,” J. Immunol. 168:6294-6297). mRNA expression of B7-H3 has been found in heart, kidney, testes, lung, liver, pancreas, prostate, colon, and osteoblast cells (Collins, M. et al. (2005) “The B7 Family Of Immune-Regulatory Ligands,” Genome Biol. 6:223.1-223.7). At the protein level, B7-H3 is found in human liver, lung, bladder, testis, prostate, breast, placenta, and lymphoid organs (Hofmeyer, K. et al. (2008) “The Contrasting Role Of B7-H3,” Proc. Natl. Acad. Sci. (U.S.A.) 105(30):10277-10278).

### II. CD3

CD3 is a T cell co-receptor composed of four distinct chains (Wucherpfennig, K. W. et al. (2010) “Structural Biology Of The T cell Receptor: Insights Into Receptor Assembly, Ligand Recognition, And Initiation Of Signaling,” Cold Spring Harb. Perspect. Biol. 2(4):a005140; pages 1-14; Chetty, R. et al. (1994) “CD3: Structure, Function, And Role Of Immunostaining In Clinical Practice,” J. Pathol. 173(4):303-307; Guy, C. S. et al. (2009) “Organization Of Proximal Signal Initiation At The TCR: CD3 Complex,” Immunol. Rev. 232(1):7-21).

In mammals, the complex contains a CD3γ chain, a CD3δ chain, and two CD3ε chains. These chains associate with a molecule known as the T cell receptor (TCR) in order to generate an activation signal in T lymphocytes (Smith-Garvin, J. E. et al. (2009) “T Cell Activation,” Annu. Rev. Immunol. 27:591-619). In the absence of CD3, TCRs do not assemble properly and are degraded (Thomas, S. et al. (2010) “Molecular Immunology Lessons From Therapeutic T cell Receptor Gene Transfer,” Immunology 129(2):170-177). CD3 is found bound to the membranes of all mature T cells, and in virtually no other cell type (see, Janeway, C. A. et al. (2005) In: IMMUNOBIOLOGY: THE IMMUNE SYSTEM IN HEALTH AND DISEASE,” 6th ed. Garland Science Publishing, NY, pp. 214-216; Sun, Z. J. et al. (2001) “Mechanisms Contributing To T Cell Receptor Signaling And Assembly Revealed By The Solution Structure Of An Ectodomain Fragment Of The CD3ε:γ Heterodimer,” Cell 105(7): 913-923; Kuhns, M. S. et al. (2006) “Deconstructing The Form And Function Of The TCR/CD3 Complex,” Immunity. 2006 February; 24(2):133-139).

The invariant CD3c signaling component of the T cell receptor (TCR) complex on T cells has been used as a target to force the formation of an immunological synapse between T cells and tumor cells. Co-engagement of CD3 and the tumor antigen activates the T cells, triggering lysis of tumor cells expressing the tumor antigen (Baeuerle et al. (2011) “Bispecific T Cell Engager For Cancer Therapy,” In: BISPECIFIC ANTIBODIES, Kontermann, R. E. (Ed.) Springer-Verlag; 2011:273-287). This approach allows bispecific antibodies to interact globally with the T cell compartment with high specificity for tumor cells and is widely applicable to a broad array of cell-surface tumor antigens.

### III. Antibodies and Other Binding Molecules

Antibodies are immunoglobulin molecules capable of specific binding to a target region (“epitope”) of a molecule, such as a carbohydrate, polynucleotide, lipid, polypeptide, etc. (“antigen”), through at least one epitope-binding site located in the Variable Region of the immunoglobulin molecule. As used herein, the term encompasses not only intact polyclonal or monoclonal antibodies, but also mutants thereof, naturally occurring variants, fusion proteins comprising an antibody portion with an epitope-binding site of the required specificity, humanized antibodies, and chimeric antibodies, and any other modified configuration of the immunoglobulin molecule that comprises an epitope-binding site of the required specificity.

The ability of an intact, unmodified antibody (e.g., an IgG antibody) to bind an epitope of an antigen depends upon the presence of Variable Domains on the immunoglobulin “light” and “heavy” polypeptide chains (i.e., the VL and VH Domains, respectively, and collectively, the “Variable Region”). Each VH and VL is composed of three Complementarity Determining Region (CDR) Domains and four FR Domains arranged from amino-terminus to carboxy-terminus in the following order: FR1-CDR1-FR2-CDR2-FR3-CDR3-FR4. The amino acid sequences of the CDRs determine whether an antibody will be able to bind to a particular epitope. Interaction of an antibody light chain with an antibody heavy chain and, in particular, interaction of their VL and VH Domains, each present on a separate polypeptide, forms one of the epitope binding sites of the antibody. In contrast, an scFv construct comprises a VL and VH Domain of an antibody contained in a single polypeptide chain, wherein the Domains are separated by a flexible linker of sufficient length to allow self-assembly of the two Domains into a functional epitope binding site. Where self-assembly of the VL and VH Domains is rendered impossible due to a linker of insufficient length (e.g., a linker of less than about 12 amino acid residues), two scFv molecules can interact with one another other to form a bivalent “diabody” molecule in which the VL of one molecule associates with the VH of the other (reviewed in Marvin et al. (2005) “Recombinant Approaches To IgG-Like Bispecific Antibodies,” Acta Pharmacol. Sin. 26:649-658).

Natural antibodies are capable of binding to only one epitope species (i.e., mono-specific), although they can bind multiple copies of that species (i.e., exhibiting bi-valency or multi-valency). A wide variety of recombinant bispecific antibody formats have been developed (see, e.g., PCT Publication Nos. WO 2008/003116, WO 2009/132876, WO 2008/003103, WO 2007/146968, WO 2009/018386, WO 2012/009544, WO 2013/070565), most of which use linker peptides either to fuse a further binding domain (e.g. an scFv, VL, VH, etc.) to, or within the antibody core (IgA, IgD, IgE, IgG or IgM), or to fuse multiple antibody binding portions to one another (e.g. two Fab fragments or scFv). Alternative formats use linker peptides to fuse a binding protein (e.g., an scFv, VL, VH, etc.) to a dimerization domain such as the CH2-CH3 Domain or alternative polypeptides (WO 2005/070966, WO 2006/107786A WO 2006/107617A, WO 2007/046893). Typically, such approaches involve compromises and trade-offs. For example, PCT Publications Nos. WO 2013/174873, WO 2011/133886 and WO 2010/136172 disclose that the use of linkers may cause problems in therapeutic settings, and teaches a tri-specific antibody in which the CL and CH1 Domains are switched from their respective natural positions and the VL and VH Domains have been diversified (WO 2008/027236; WO 2010/108127) to allow them to bind to more than one antigen. Thus, the molecules disclosed in these documents trade binding specificity for the ability to bind additional antigen species. PCT Publications Nos. WO 2013/163427 and WO 2013/119903 disclose modifying the CH2 Domain to contain a fusion protein adduct comprising a binding domain. The document notes that the CH2 Domain likely plays only a minimal role in mediating effector function. PCT Publications Nos. WO 2010/028797, WO2010028796 and WO 2010/028795 disclose recombinant antibodies whose Fc Regions have been replaced with additional VL and VH Domains, so as to form tri-valent binding molecules. PCT Publications Nos. WO 2003/025018 and WO2003012069 disclose recombinant diabodies whose individual chains contain scFv domains. PCT Publications No. WO 2013/006544 discloses multi-valent Fab molecules that are synthesized as a single polypeptide chain and then subjected to proteolysis to yield heterodimeric structures. Thus, the molecules disclosed in these documents trade all or some of the capability of mediating effector function for the ability to bind additional antigen species. PCT Publications Nos. WO 2014/022540, WO 2013/003652, WO 2012/162583, WO 2012/156430, WO 2011/086091, WO 2008/024188, WO 2007/024715, WO 2007/075270, WO 1998/002463, WO 1992/022583 and WO 1991/003493 disclose adding additional Binding Domains or functional groups to an antibody or an antibody portion (e.g., adding a diabody to the antibody's light chain, or adding additional VL and VH Domains to the antibody's light and heavy chains, or adding a heterologous fusion protein or chaining multiple Fab Domains to one another). Thus, the molecules disclosed in these documents trade native antibody structure for the ability to bind additional antigen species.

The art has additionally noted the capability to produce diabodies that differ from natural antibodies in being capable of binding two or more different epitope species (i.e., exhibiting bispecificity or multispecificity in addition to bi-valency or multi-valency) (see, e.g., Holliger et al. (1993) “‘Diabodies’: Small Bivalent And Bispecific Antibody Fragments,” Proc. Natl. Acad. Sci. (U.S.A.) 90:6444-6448; US 2004/0058400 (Hollinger et al.); US 2004/0220388 (Mertens et al.); Alt et al. (1999) FEBS Lett. 454(1-2):90-94; Lu, D. et al. (2005) “A Fully Human Recombinant IgG-Like Bispecific Antibody To Both The Epidermal Growth Factor Receptor And The Insulin-Like Growth Factor Receptor For Enhanced Antitumor Activity,” J. Biol. Chem. 280(20):19665-19672; WO 02/02781 (Mertens et al.); Olafsen, T. et al. (2004) “Covalent Disulfide-Linked Anti-CEA Diabody Allows Site-Specific Conjugation And Radiolabeling For Tumor Targeting Applications,” Protein Eng Des Sel. 17(1):21-27; Wu, A. et al. (2001) “Multimerization Of A Chimeric Anti-CD20 Single Chain Fv-Fv Fusion Protein Is Mediated Through Variable Domain Exchange,” Protein Engineering 14(2):1025-1033; Asano et al. (2004) “A Diabody For Cancer Immunotherapy And Its Functional Enhancement By Fusion Of Human Fc Domain,” Abstract 3P-683, J. Biochem. 76(8):992; Takemura, S. et al. (2000) “Construction Of A Diabody (Small Recombinant Bispecific Antibody) Using A Refolding System,” Protein Eng. 13(8):583-588; Baeuerle, P. A. et al. (2009) “Bispecific T cell Engaging Antibodies For Cancer Therapy,” Cancer Res. 69(12):4941-4944).

In particular, stable, covalently bonded heterodimeric non-monospecific diabodies have been described (see, e.g., Chichili, G. R. et al. (2015) “A CD3×CD123 Bispecific DART For Redirecting Host T Cells To Myelogenous Leukemia: Preclinical Activity And Safety In Nonhuman Primates,” Sci. Transl. Med. 7(289):289ra82; Johnson, S. et al. (2010) “Effector Cell Recruitment With Novel Fv-Based Dual-Affinity Re-Targeting Protein Leads To Potent Tumor Cytolysis And In Vivo B-Cell Depletion,” J. Molec. Biol. 399(3):436-449; Veri, M. C. et al. (2010) “Therapeutic Control Of B Cell Activation Via Recruitment Of Fcgamma Receptor IIb (CD32B) Inhibitory Function With A Novel Bispecific Antibody Scaffold,” Arthritis Rheum. 62(7):1933-1943; Moore, P. A. et al. (2011) “Application Of Dual Affinity Retargeting Molecules To Achieve Optimal Redirected T cell Killing Of B-Cell Lymphoma,” Blood 117(17):4542-4551; U.S. Pat. Nos. 8,044,180; 8,133,982; 8,187,593; 8,193,318; 8,530,627; 8,669,349; 8,778,339; 8,784,808; 8,795,667; 8,802,091; 8,802,093; 8,946,387; 8,968,730; and 8,993,730; US Patent Publications No. 2009/0060910; 2010/0174053; 2011/0081347; 2011/0097323; 2011/0117089; 2012/0009186; 2012/0034221; 2012/0141476; 2012/0294796; 2013/0149236; 2013/0295121; 2014/0017237; and 2014/0099318; European Patent Documents No. EP 1868650; EP 2158221; EP 2247304; EP 2252631; EP 2282770; EP 2328934; EP 2376109; EP 2542256; EP 2601216; EP 2714079; EP 2714733; EP 2786762; EP 2839842; EP 2840091; and PCT Publications No. WO 2006/113665; WO 2008/157379; WO 2010/027797; WO 2010/033279; WO 2010/080538; WO 2011/109400; WO 2012/018687; WO 2012/162067; WO 2012/162068; WO 2014/159940; WO 2015/021089; WO 2015/026892; and WO 2015/026894).

Notwithstanding such success, the production of stable, functional heterodimeric, non-monospecific diabodies optimized for therapeutic use can be further improved by the careful consideration and placement of the domains employed in the polypeptide chains. The present invention is thus directed to the provision of specific polypeptides that are particularly designed to form, via covalent bonding, stable and therapeutically useful heterodimeric diabodies and heterodimeric Fc diabodies that are capable of simultaneously binding B7-H3 and CD3.

### SUMMARY OF THE INVENTION

The present invention is directed to bispecific monovalent diabodies that possess one binding site specific for an epitope of B7-H3 and one binding site specific for an epitope of CD3 (i.e., a “B7-H3×CD3 bispecific monovalent diabody”). Most preferably, such B7-H3×CD3 bispecific monovalent diabodies are composed of three polypeptide chains and possess one binding site specific for an epitope of B7-H3 and one binding site specific for an epitope of CD3 and additionally comprise an immunoglobulin Fc Domain (i.e., a “B7-H3×CD3 bispecific monovalent Fc diabody”). The bispecific monovalent Fc diabodies of the present invention are capable of simultaneous binding to B7-H3 and CD3. The invention is directed to pharmaceutical compositions that contain such bispecific monovalent Fc diabodies. The invention is additionally directed to methods for the use of such diabodies in the treatment of cancer and other diseases and conditions.

The present invention is particularly directed to B7-H3×CD3 bispecific monovalent Fc diabodies. The B7-H3×CD3 bispecific monovalent Fc diabodies of the invention comprise polypeptide chains that associate with one another in a heterodimeric manner to form one binding site specific for an epitope of B7-H3 and one binding site specific for an epitope of CD3. The B7-H3×CD3 bispecific monovalent Fc diabodies of the invention are thus monovalent in that they are capable of binding to only one copy of an epitope of B7-H3 and to only one copy of an epitope of CD3, but bispecific in that a single diabody is able to bind simultaneously to the epitope of B7-H3 and to the epitope of CD3.

The preferred B7-H3×CD3 bispecific monovalent Fc diabodies of the invention comprise three polypeptide chains (a “first,” “second” and “third” polypeptide chain), wherein the first and second polypeptide chains are covalently bonded to one another and the first and third polypeptide chains are covalently bonded to one another.

In detail, the invention provides a B7-H3×CD3 bispecific monovalent Fc diabody, wherein the bispecific monovalent Fc diabody is capable of specific binding to an epitope of B7-H3 and to an epitope of CD3, and possesses an IgG Fc Domain, wherein the bispecific monovalent Fc diabody comprises a first polypeptide chain, a second polypeptide chain and a third polypeptide chain, wherein the first and second polypeptide chains are covalently bonded to one another and the first and third polypeptide chains are covalently bonded to one another, and wherein:
I. the first polypeptide chain comprises, in the N-terminal to C-terminal direction:
   A. a Domain IA, comprising:
      (1) a sub-Domain (IA1), which comprises a VL Domain capable of binding to either B7-H3 (VL_{B7-H3}) or CD3 (VL_{CD3}); and
      (2) a sub-Domain (IA2), which comprises a VH Domain capable of binding to either B7-H3 (VH_{B7-H3}) or CD3 (VH_{CD3});
      wherein the sub-Domains IA1 and IA2 are separated from one another by a polypeptide linker of 12 or less amino acid residues, and are coordinately selected, such that:
      (a) the sub-Domain IA1 comprises the VL Domain capable of binding to B7-H3 (VL_{B7-H3}) and the sub-Domain IA2 is selected to comprise the VH Domain capable of binding to CD3 (VH_{CD3}); or
      (b) the sub-Domain IA1 comprises the VL Domain capable of binding to CD3 (VL_{CD3}) and the sub-Domain IA2 is selected to comprise the VH Domain capable of binding to B7-H3 (VH_{B7-H3}); and
   B. an optionally present Domain IB, comprising a polypeptide linker linked to a Heterodimer-Promoting Domain;
   C. a Domain IC, comprising a polypeptide linker linked to a CH2-CH3 Domain of an antibody;
II. the second polypeptide chain comprises, in the N-terminal to C-terminal direction:
   A. a Domain IIA, comprising:
      (1) a sub-Domain (IIA1), which comprises a VL Domain capable of binding to either B7-H3 (VL_{B7-H3}) or CD3 (VL_{CD3}); and
      (2) a sub-Domain (IIA2), which comprises a VH Domain capable of binding to either B7-H3 (VH_{B7-H3}) or CD3 (VH_{CD3});
      wherein the sub-Domains IIA1 and IIA2 are separated from one another by a polypeptide linker of 12 or less amino acid residues, and are coordinately selected, such that:
         (a) when the sub-Domain IA1 comprises the VL Domain capable of binding to B7-H3 (VL_{B7-H3}), the sub-Domain IIA1 is selected to comprise the VL Domain capable of binding to CD3 (VL_{CD3}) and the sub-Domain IIA2 is selected to comprise the VH Domain capable of binding to B7-H3 (VH_{B7-H3}); and
         (b) when the sub-Domain IA1 comprises the VL Domain capable of binding to CD3 (VL_{CD3}), then sub-Domain IIA1 is selected to comprise the VL Domain capable of binding to B7-H3 (VL_{B7-H3}) and the sub-Domain IIA2 is selected to comprise the VH Domain capable of binding to CD3 (VH_{CD3});
   B. an optionally present Domain IIB, comprising a polypeptide linker linked to a Heterodimer-Promoting Domain;
III. the third polypeptide chain comprises, in the N-terminal to C-terminal direction a Domain IIIC that comprises a polypeptide linker linked to a CH2-CH3 Domain of an antibody;
   wherein:
(A) (1) at least one of the optionally present Domain IB and the optionally present Domain IIB is present, and wherein the Domain IB or IIB that is present has a positive or negative charge; or
   (2) both the optionally present Domain IB and the optionally present Domain IIB are present, wherein:
      (i) one of the Domain 1B and the Domain IIB has a Heterodimer-Promoting Domain having a positive charge, and the other of the Domain 1B and the Domain IIB has a Heterodimer-Promoting Domain having a negative charge; or
      (ii) one of the Domain 1B and the Domain IIB has a Heterodimer-Promoting Domain that comprises the amino acid sequence GVEPKSC (SEQ ID NO:6) or VEPKSC (SEQ ID NO:7), and the other of the Domain 1B and the Domain IIB has a Heterodimer-Promoting Domain that comprises the amino acid sequence GFNRGEC (SEQ ID NO:8) or FNRGEC (SEQ ID NO:9);
(B) the VL_{B7-H3} and the VH_{B7-H3} interact to form an epitope-binding domain capable of binding an epitope of B7-H3, and the VL_{CD3} and VH_{CD3} form an epitope-binding domain capable of binding an epitope of CD3; and
(C) the CH2-CH3 Domains of the first and third polypeptide chains form an Fc Domain capable of binding to an Fc receptor.

The invention further concerns the embodiments of such B7-H3×CD3 bispecific monovalent Fc diabodies, which are capable of cross-reacting with both human and primate B7-H3 and CD3.

The invention particularly concerns the embodiments of such B7-H3×CD3 bispecific monovalent Fc diabodies wherein:
(A) the first polypeptide chain has the amino acid sequence of SEQ ID NO:53, the second polypeptide chain has the amino acid sequence of SEQ ID NO:55, and the third polypeptide chain has the amino acid sequence of SEQ ID NO:57; or
(B) the first polypeptide chain has the amino acid sequence of SEQ ID NO:59, the second polypeptide chain has the amino acid sequence of SEQ ID NO:60 and the third polypeptide chain has the amino acid sequence of SEQ ID NO:57; or
(C) the first polypeptide chain has the amino acid sequence of SEQ ID NO:61, the second polypeptide chain has the amino acid sequence of SEQ ID NO:62, and the third polypeptide chain has the amino acid sequence of SEQ ID NO:57
(D) the first polypeptide chain has the amino acid sequence of SEQ ID NO:63, the second polypeptide chain has the amino acid sequence of SEQ ID NO:64, and the third polypeptide chain has the amino acid sequence of SEQ ID NO:57.

The B7-H3×CD3 bi-specific monovalent Fc diabodies of the present invention are preferably capable of mediating redirected killing of target tumor cells using human T cells in an assay employing a target human tumor cell line selected from the group consisting of: A498 (kidney cancer), JIMT-1/Luc (breast cancer), A375 (melanoma); 22Rv1 (prostate cancer), Detroit562 (pharyngeal cancer), DU145 (prostate cancer); BxPC3 (pancreatic cancer), SKMES-1 (lung cancer), and U87 (glioblastoma), and using purified human primary T cells as effector cells at an Effector cell to T cell ratio of 1:1, 5:1, or 10:1. In such an assay, target tumor cell killing is measured using a lactate dehydrogenase (LDH) release assay in which the enzymatic activity of LDH released from cells upon cell death is quantitatively measured, or by a luciferase assay in which luciferase relative light unit (RLU) is the read-out to indicate relative viability of target cells, which have been engineered to express both the green fluorescent protein (GFP) and luciferase reporter genes. The observed EC₅₀ of such redirected killing is about 1.5 μg/mL or less, about 1.0 μg/mL or less, about 500 ng/mL or less, about 300 ng/mL or less, about 200 ng/mL or less, about 100 ng/mL or less, about 50 ng/mL or less.

The B7-H3×CD3 bi-specific monovalent Fc diabodies of the present invention are preferably capable of mediating the inhibition of human tumor growth in a co-mix xenograft in which such molecules are introduced into NOD/SCID mice along with 22Rv1 (human prostate cancer) or A498 (human kidney cancer) tumor cells and activated human T cells at a ratio of 5:1. Additionally, or alternatively the B7-H3×CD3 bi-specific monovalent Fc diabodies of the present invention are capable of mediating the inhibition of human tumor growth and/or exhibiting anti-tumor activity in a in an xenograft model in female NSG B2m −/− mice:
(A) implanted with human PBMCs (1×10⁷) by intraperitoneal (IP) injection on Day −1 and Detroit (human pharyngeal cancer tumor cells (5×10⁶)) intradermally (ID) on Day 0, and administration of diabody on or Days 20, 22, 23, 26, 28, 30, 33, 35, and 37; or
(B) implanted with A498 (human kidney cancer tumor cells (5×10⁶)) intradermally (ID) on Day 0, and human PBMCs (1×10⁷) by intraperitoneal (IP) injection on Day 13 and administration of diabody on Days 33, 35, 36, 39, 41, 43, 46, 48, and 50.

Preferably, the B7-H3×CD3 bi-specific monovalent Fc diabodies of the present invention are capable of inhibiting tumor growth in such xenograft models when provided at a concentration of greater than about 1.0 mg/kg, at a concentration of about 1 mg/kg, at a concentration of about 0.5 mg/kg, at a concentration of about 0.25 mg/kg, at a concentration of about 0.1 mg/kg, at a concentration of about 0.05 mg/kg, at a concentration of about 0.02 mg/kg, at a concentration of about 0.01 mg/kg, or at a concentration of about 0.005 mg/kg, or at a concentration less than 0.005 mg/kg.

The invention additionally provides any of the above-described B7-H3×CD3 bi-specific monovalent Fc diabodies for use as a pharmaceutical.

The invention additionally provides any of the above-described B7-H3×CD3 bi-specific monovalent Fc diabodies for use in the treatment of a disease or condition associated with or characterized by the expression of B7-H3, or in a method of treating a disease or condition characterized by the expression of B7-H3, particularly wherein the disease or condition associated with or characterized by the expression of B7-H3 is cancer, and more particularly, wherein the cancer is selected from the group consisting of: an acute myeloid leukemia, an adrenal gland tumor, an AIDS-associated cancer, an alveolar soft part sarcoma, an astrocytic tumor, bladder cancer, bone cancer, a brain and spinal cord cancer, a metastatic brain tumor, a breast cancer, a carotid body tumors, a cervical cancer, a chondrosarcoma, a chordoma, a chromophobe renal cell carcinoma, a clear cell carcinoma, a colon cancer, a colorectal cancer, a cutaneous benign fibrous histiocytoma, a desmoplastic small round cell tumor, an ependymoma, a Ewing's tumor, an extraskeletal myxoid chondrosarcoma, a fibrogenesis imperfecta ossium, a fibrous dysplasia of the bone, a gallbladder or bile duct cancer, gastric cancer, a gestational trophoblastic disease, a germ cell tumor, a head and neck cancer, hepatocellular carcinoma, a glioblastoma, an islet cell tumor, a Kaposi's Sarcoma, a kidney cancer, a leukemia, a lipoma/benign lipomatous tumor, a liposarcoma/malignant lipomatous tumor, a liver cancer, a lymphoma, a lung cancer, a medulloblastoma, a melanoma, a meningioma, a malignant mesothelioma, a multiple endocrine neoplasia, a multiple myeloma, a myelodysplastic syndrome, a neuroblastoma, a neuroendocrine tumors, a non-small cell lung cancer, an ovarian cancer, a pancreatic cancer, a pharyngeal cancer, a papillary thyroid carcinoma, a parathyroid tumor, a pediatric cancer, a peripheral nerve sheath tumor, a phaeochromocytoma, a pituitary tumor, a prostate cancer, a posterious uveal melanoma, a rare hematologic disorder, a renal cell carcinoma, a renal metastatic cancer, a rhabdoid tumor, a rhabdomysarcoma, a sarcoma, a skin cancer, a soft-tissue sarcoma, a squamous cell cancer, a stomach cancer, a synovial sarcoma, a testicular cancer, a thymic carcinoma, a thymoma, a thyroid metastatic cancer, and a uterine cancer.

### BRIEF DESCRIPTION OF THE DRAWINGS

FIG. 1 illustrates the structure of a covalently associated bispecific monovalent diabody composed of two polypeptide chains, which does not comprise an Fc Region. The polypeptide chains are covalently associated to one another via disulfide bonds that form between cysteine (“C”) residues.

FIGS. 2A and 2B illustrate the structures of two versions of the first, second and third polypeptide chains of a three chain bispecific monovalent Fc diabody of the present invention (Version 1, FIG. 2A; Version 2, FIG. 2B). The polypeptide chains are covalently associated to one another via disulfide bonds that form between cysteine (“C”) residues

FIGS. 3A-3J show FACS histograms of A498 (kidney cancer) (FIG. 3A), JIMT-1/Luc (breast cancer) (FIG. 3B), A375 (melanoma) (FIG. 3C), 22Rv1 (prostate cancer) (FIG. 1D), Detroit562 (pharyngeal cancer) (FIG. 1E), DU145 (prostate cancer) (FIG. 3F), BxPC-3 (pancreatic cancer) (FIG. 3G), SKMES-1 (lung cancer) (FIG. 311), U87 (glioblastoma) (FIG. 3I), and Raji (B-lymphoma) (FIG. 3J) cell lines. Dashed lines represent cells stained with an isotype control PE-labeled antibody and solid lines represent cells stained with anti-B7-H3-PE antibody.

FIGS. 4A-4E show FACS histograms of anti-EK-coil antibody fluorescence on B7-H3-expressing target cancer cell lines (FIGS. 4A-4D) or human primary T cells (FIG. 4E). DART-A at a concentration of 10 μg/mL was added to B7-H3-expressing cancer cell lines (A498 (FIG. 4A), JIMT-1/Luc (FIG. 4B), Detroit562 (FIG. 4C), or 22Rv1 (FIG. 4D)) or human primary T cells (FIG. 4E) and incubated for 30 minutes. Cells were analyzed by FACS for DART-A cell surface binding after further incubation with biotin-conjugated anti-EK-coil antibody mixed with APC-streptavidin. Solid lines represent DART-A binding profile on cells. Non-specific staining on cells from biotin-conjugated anti-EK-coil antibody is shown by the dashed lines.

FIGS. 5A-5L show dose-response curves for DART-A-mediated cytotoxicity on B7-H3-expressing cell lines (A498 (FIG. 5A), JIMT-1/Luc (FIGS. 5B-5C), A375 (FIG. 5D), U87 (FIG. 5E), DU145 (FIG. 5F), BxPC-3 (FIG. 5G), SKMES-1 (FIG. 511), Detroit562 (FIG. 5I) and 22Rv1 (FIG. 5J)) and B7-H3-negative cell lines (CHO (FIG. 5K) and Raji (FIG. 5L)). DART-A or control DART was incubated in vitro with the different tumor cell lines and primary human T cells at an effector cell:target cell (E:T) ratio of 5:1 for about 24 hours. Percent cytotoxicity was evaluated using the LDH release assay for all cell lines (FIGS. 5A-5B and 5D-5L). In addition, cytotoxicity was measured using the LUM assay for the JIMT-1/Luc cell line (FIG. 5C). Representative data are shown from multiple experiments using T cells from multiple donors. DART-A: ●; Control DART: ▪.

FIGS. 6A-6F show DART-A-mediated redirected killing of A498 cells (FIGS. 6A, 6C and 6E) and A375 cells (FIGS. 6B, 6D and 6F) at E:T ratios of 10:1 (FIGS. 6A and 6B), 5:1 (FIGS. 6C and 6D) and 1:1 (FIGS. 6E and 6F). Cytotoxicity was determined by LDH assay. DART-A: ●; Control DART: ▪.

FIGS. 7A-7E show dose-response curves of DART-A-mediated redirected target cell killing (FIG. 7A) and induction of T cell activation markers CD25 (FIGS. 7B and 7C) and CD69 (FIGS. 7D and 7E) on CD4+(FIGS. 7B and 7D) and CD8+(FIGS. 7C and 7E) T cells following incubation with purified T cells as effector cells and A498 target cells at an E:T cell ratio of 10:1 for 24 hours. DART-A: ●; Control DART: ▪. T cells +A498 cells: ▾; T cells alone ▴.

FIGS. 8A-8B show DART-A-mediated T cell proliferation in the presence of B7-H3-positive target cells. Proliferation of human primary T cells was evaluated by FACS analysis after co-culturing of CFSE-labeled human primary T cells with A498 target cells at an E:T ratio of 10:1 in the presence of DART-A (heavy line) or Control DART (thin line filled) at 10 μg/mL for 72 hours (FIG. 8A) or 96 hours (FIG. 8B).

FIG. 9A-9D show DART-A efficiently binds to human (FIG. 9A) and cynomolgus monkey (FIG. 9B) B7-H3-expressing CHO cells and mediates redirected killing of the human (FIG. 9C) and cynomolgus monkey (FIG. 9D) B7-H3-expressing CHO cells following incubation with purified human primary T cells as effector cells and B7-H3-expressing CHO target cells at an E:T cell ratio of 5:1 for 24 hours. Cytotoxicity was measured using the LDH assay. DART-A: ●; Control DART: ▪.

FIGS. 10A-10B show that DART-A is capable of binding to cynomolgus monkey and human primary T cells. DART-A at 10 μg/mL was added to cynomolgus monkey (FIG. 10A) or human (FIG. 10B) PBMCs and cells were incubated for 30 minutes at 4° C. followed by a second incubation with biotin-conjugated anti-EK-coil antibody mixed with APC-streptavidin. Cells were analyzed by FACS for DART-A T cell surface binding (thick lines) on gated total combined CD4+ and CD8+ cells. Non-specific staining on cells from biotin-conjugated anti-EK-coil secondary antibody is shown by the thin line/thin line with shading.

FIGS. 11A-11C show DART-A-mediated redirected killing of B7-H3-positive target cell lines JIMT-1/Luc (FIGS. 11A and 11B) and A498 (FIG. 11C) using cynomolgus monkey PBMCs at an E:T ratio of 30:1. Cytotoxicity was measured using the LUM assay (FIG. 11A) or the LDH assay (FIGS. 11B and 11C). DART-A: ●; Control DART: ▪.

FIG. 12 shows the inhibition of tumor growth by DART-A in mice implanted with 22Rv1 tumor cells in the presence of activated human T cells. Female NOD/SCID mice (n=8/group) were implanted SC with 22Rv1 tumor cells (5×10⁶) co-mixed with activated human T cells (1×10⁶) on Day 0 followed by treatment with vehicle control (●), Control DART (∘), or DART-A at 0.5 mg/kg (▪), 0.1 mg/kg (▴), 0.02 mg/kg (▾) or 0.004 mg/kg (♦) on Days 0, 1, 2 and 3 for a total of 4 doses administered IV. Tumor volume is shown as group mean±SEM.

FIG. 13 shows the inhibition of tumor growth by DART-A in mice implanted with A498 tumor cells in the presence of activated human T cells. Female NOD/SCID mice (n=8/group) were implanted SC with A498 tumor cells (5×10⁶) co-mixed with activated human T cells (1×10⁶) on Day 0 followed by treatment with vehicle control (●), Control DART at 0.5 mg/kg (∘), or DART-A at 0.5 mg/kg (▪), 0.1 mg/kg (▴), 0.02 mg/kg (▾) or 0.004 mg/kg (♦) on Days 0, 1, 2 and 3 for a total of 4 doses administered IV. Tumor volume is shown as group mean±SEM.

FIG. 14 shows the anti-tumor activity of DART-A in NSG B2m−/− mice implanted with A498 tumor cells and reconstituted with human effector cells. Female NSG B2m−/− mice (n=7/group) were implanted ID with A498 tumor cells (5×10⁶ cells) on Day 0 followed by human PBMCs (1×10⁷) implanted IP on Day 13. The groups were then treated with vehicle control (●), Control DART at 0.5 mg/kg (◯), or DART-A at 1 mg/kg (▪), 0.1 mg/kg (▴), 0.01 mg/kg (▾) or 0.001 mg/kg (♦) on Days 33, 35, 36, 39, 41, 43, 46, 48, and 50. Tumor volume is shown as group mean±SEM.

FIG. 15 shows the anti-tumor activity of DART-A in NSG B2m−/− mice implanted with Detroit562 tumor cells and reconstituted with human effector cells. Female NSG B2m−/− mice (n=8/group) were implanted ID with Detroit562 tumor cells (5×10⁶ cells) on Day 0, while human PBMCs (1×10⁷) were implanted IP on Day −1. Subsequently the groups were treated with vehicle control (●), Control DART at 0.5 mg/kg (◯) or DART-A at 1 mg/kg (▪), 0.5 mg/kg (▴), 0.25 mg/kg (▾) or 0.1 mg/kg (♦) for a total of 9 doses administered IV on Days 20, 22, 23, 26, 28, 30, 33, 35, and 37. Tumor volume is shown as group mean±SEM.

FIGS. 16A-16B show the anti-tumor activity of DART-A in NSG MHCl1 −/− mice implanted with Detroit562 tumor cells and reconstituted with human effector cells. On Day 0 MHCl1 −/− mice (n=7/vehicle control; 7/Group I; and 5/Group II) were implanted ID with Detroit562 tumor cells (5×10⁶ cells) and were implanted IP with human PBMCs (1×10⁷ cells). Subsequently the groups were treated with vehicle control (●), or DART-A at 0.5 mg/kg (▴). Group I (FIG. 16A) received 5 doses administered IV on Days 15, 22, 29, 36 and 43. Group II (FIG. 16B) received 3 doses administered IV on Days 15, 29 and 43. Tumor volume is shown as the group mean±SEM.

FIG. 17 shows the pharmacokinetic profiles of DART-A and DART-B in cynomolgus monkeys. Groups of cynomolgus monkeys (n=2/group; 1M/1F) were administered one dose of 0.5 mg/kg then three weekly doses of 1.0 mg/kg of DART-A or DART-B for a total of four doses administered IV. The serum concentration of DART-A (solid lines) and DART-B (dashed lines) for each of the four test animals over the course of the study are plotted.

### DETAILED DESCRIPTION OF THE INVENTION

The present invention is directed to bispecific monovalent diabodies that possess one binding site specific for an epitope of B7-H3 and one binding site specific for an epitope of CD3 (i.e., a “B7-H3×CD3 bispecific monovalent diabody”). Most preferably, such B7-H3×CD3 bispecific monovalent diabodies are composed of three polypeptide chains and possess one binding site specific for an epitope of B7-H3 and one binding site specific for an epitope of CD3 and additionally comprise an immunoglobulin Fc Domain (i.e., a “B7-H3×CD3 bispecific monovalent Fc diabody”). The bispecific monovalent Fc diabodies of the present invention are capable of simultaneous binding to B7-H3 and CD3. The invention is directed to pharmaceutical compositions that contain such bispecific monovalent Fc diabodies. The invention is additionally directed to methods for the use of such diabodies in the treatment of cancer and other diseases and conditions.

### I. Antibodies and Other Binding Molecules

A. Antibodies

As used herein, the term “antibodies” encompasses any molecule possessing an immunoglobulin Variable Domain capable of immunospecifically binding to an epitope (an “an epitope-binding site”). The term thus encompasses not only intact polyclonal or monoclonal antibodies, but also mutants thereof, naturally occurring variants, fusion proteins comprising such epitope-binding site, humanized antibodies and chimeric antibodies, and any other modified configuration of the immunoglobulin molecule capable of immunospecifically binding to an epitope. Throughout this application, the numbering of amino acid residues of the constant regions of the light and heavy chains of antibodies is according to the EU index as in Kabat et al. (1992) SEQUENCES OF PROTEINS OF IMMUNOLOGICAL INTEREST, National Institutes of Health Publication No. 91-3242. As used herein, an “epitope-binding fragment of an antibody” is intended to denote a portion of an antibody capable of immunospecifically binding to an epitope. As used herein, such term encompasses fragments (such as Fab, Fab′, F(ab′)₂ Fv), and single chain (scFv), as well as the epitope-binding domain of a diabody.

The term “monoclonal antibody” refers to a homogeneous antibody population capable of immunospecifically binding to an epitope. The term “monoclonal antibody” is not intended to be limited as regards to the source of the antibody or the manner in which it is made (e.g., by hybridoma, phage selection, recombinant expression, production in transgenic animals, etc.). Methods of making monoclonal antibodies are known in the art. One method which may be employed is the method of Kohler, G. et al. (1975) “Continuous Cultures Of Fused Cells Secreting Antibody Of Predefined Specificity,” Nature 256:495-497 or a modification thereof. Typically, monoclonal antibodies are developed in mice, rats or rabbits. The antibodies are produced by immunizing an animal with an immunogenic amount of cells, cell extracts, or protein preparations that contain the desired epitope. The immunogen can be, but is not limited to, primary cells, cultured cell lines, cancerous cells, proteins, peptides, nucleic acids, or tissue. Cells used for immunization may be cultured for a period of time (e.g., at least 24 hours) prior to their use as an immunogen. Cells may be used as immunogens by themselves or in combination with a non-denaturing adjuvant, such as Ribi. In general, cells should be kept intact and preferably viable when used as immunogens. Intact cells may allow antigens to be better detected than ruptured cells by the immunized animal. Use of denaturing or harsh adjuvants, e.g., Freund's adjuvant, may rupture cells and therefore is discouraged. The immunogen may be administered multiple times at periodic intervals such as, bi weekly, or weekly, or may be administered in such a way as to maintain viability in the animal (e.g., in a tissue recombinant). Alternatively, existing monoclonal antibodies and any other equivalent antibodies that are immunospecific for a desired pathogenic epitope can be sequenced and produced recombinantly by any means known in the art. In one embodiment, such an antibody is sequenced and the polynucleotide sequence is then cloned into a vector for expression or propagation. The sequence encoding the antibody of interest may be maintained in a vector in a host cell and the host cell can then be expanded and frozen for future use. The polynucleotide sequence of such antibodies may be used for genetic manipulation to generate the bispecific molecules of the invention as well as a chimeric antibody, a humanized antibody, or a caninized antibody, to improve the affinity, or other characteristics of the antibody. The general principle in humanizing an antibody involves retaining the basic sequence of the epitope-binding portion of the antibody, while swapping the non-human remainder of the antibody with human antibody sequences. There are four general steps to humanize a monoclonal antibody. These are: (1) determining the nucleotide and/or predicted amino acid sequence of the light and heavy chain Variable Domains of the starting antibody; (2) designing the humanized antibody or caninized antibody, i.e., deciding which antibody framework region(s) to use during the humanizing or caninizing process; (3) application of the actual humanizing or caninizing methodologies/techniques; and (4) the transfection and expression of the humanized or caninized antibody (see, for example, U.S. Pat. Nos. 4,816,567; 5,807,715; 5,866,692; and 6,331,415).

As used herein, an antibody or an epitope-binding fragment thereof is said to “immunospecifically” bind a region of another molecule (i.e., an epitope) if it reacts or associates more frequently, more rapidly, with greater duration and/or with greater affinity or avidity with that epitope relative to alternative epitopes. It is also understood by reading this definition that, for example, an antibody or an epitope-binding fragment thereof that immunospecifically binds to a first target may or may not specifically or preferentially bind to a second target.

B. Bispecific Antibodies, Multi-Specific Diabodies and DART® Diabodies

The provision of non-mono-specific “diabodies” provides a significant advantage over antibodies: the capacity to co-ligate and co-localize cells that express different epitopes. Bispecific diabodies thus have wide-ranging applications including therapy and immunodiagnosis. Bispecificity allows for great flexibility in the design and engineering of the diabody in various applications, providing enhanced avidity to multimeric antigens, the cross-linking of differing antigens, and directed targeting to specific cell types relying on the presence of both target antigens. Due to their bivalency, low dissociation rates and rapid clearance from the circulation (for diabodies of small size, at or below ˜50 kDa), diabody molecules known in the art have also shown particular use in the field of tumor imaging (Fitzgerald et al. (1997) “Improved Tumour Targeting By Disulphide Stabilized Diabodies Expressed In Pichia pastoris,” Protein Eng. 10:1221). Of particular importance is the co-ligating of differing cells, for example, the cross-linking of cytotoxic T cells to tumor cells (Staerz et al. (1985) “Hybrid Antibodies Can Target Sites For Attack By T Cells,” Nature 314:628-631, and Holliger et al. (1996) “Specific Killing Of Lymphoma Cells By Cytotoxic T cells Mediated By A Bispecific Diabody,” Protein Eng. 9:299-305) to thereby co-localize T cells to the sites of tumor cells.

Alternatively to targeting such diabodies to bind to T cells, diabody epitope binding domains may be directed to a surface determinant of a B cell, such as CD19, CD20, CD22, CD30, CD37, CD40, and CD74 (Moore, P. A. et al. (2011) “Application Of Dual Affinity Retargeting Molecules To Achieve Optimal Redirected T cell Killing Of B-Cell Lymphoma,” Blood 117(17):4542-4551; Cheson, B. D. et al. (2008) “Monoclonal Antibody Therapy For B-Cell Non Hodgkin's Lymphoma,” N. Engl. J. Med. 359(6):613-626; Castillo, J. et al. (2008) “Newer Monoclonal Antibodies For Hematological Malignancies,” Exp. Hematol. 36(7):755-768). In many studies, diabody binding to effector cell determinants, e.g., Fcγ receptors (FcγR), was also found to activate the effector cell (Holliger et al. (1996) “Specific Killing Of Lymphoma Cells By Cytotoxic T cells Mediated By A Bispecific Diabody,” Protein Eng. 9:299-305; Holliger et al. (1999) “Carcinoembryonic Antigen (CEA)-Specific T cell Activation In Colon Carcinoma Induced By Anti-CD3×Anti-CEA Bispecific Diabodies And B7×Anti-CEA Bispecific Fusion Proteins,” Cancer Res. 59:2909-2916; WO 2006/113665; WO 2008/157379; WO 2010/080538; WO 2012/018687; WO 2012/162068). Normally, effector cell activation is triggered by the binding of an antigen-bound antibody to an effector cell via an Fc Domain-FcγR interaction; thus, in this regard, diabody molecules may exhibit Ig-like functionality independent of whether they comprise an Fc Domain (e.g., as assayed in any effector function assay known in the art or exemplified herein (e.g., ADCC assay)). By cross-linking tumor and effector cells, the diabody not only brings the effector cell within the proximity of a tumor cell but leads to effective tumor killing (see e.g., Cao et al. (2003) “Bispecific Antibody Conjugates In Therapeutics,” Adv. Drug. Deliv. Rev. 55:171-197).

However, the above advantages come at a salient cost. The formation of such non-mono-specific diabodies requires the successful assembly of two or more distinct and different polypeptides (i.e., such formation requires that the diabodies be formed through the heterodimerization of different polypeptide chain species). This fact is in contrast to mono-specific diabodies, which are formed through the homodimerization of identical polypeptide chains. Because at least two dissimilar polypeptides (i.e., two polypeptide species) must be provided in order to form a non-mono-specific diabody, and because homodimerization of such polypeptides leads to inactive molecules (Takemura, S. et al. (2000) “Construction Of A Diabody (Small Recombinant Bispecific Antibody) Using A Refolding System,” Protein Eng. 13(8):583-588), the production of such polypeptides must be accomplished in such a way as to address covalent bonding between polypeptides of the same species (i.e., so as to minimize their homodimerization) (Takemura, S. et al. (2000) “Construction Of A Diabody (Small Recombinant Bispecific Antibody) Using A Refolding System,” Protein Eng. 13(8):583-588). The art has therefore taught the non-covalent association of such polypeptides (see, e.g., Olafsen et al. (2004) “Covalent Disulfide-Linked Anti-CEA Diabody Allows Site-Specific Conjugation And Radiolabeling For Tumor Targeting Applications,” Prot. Engr. Des. Sel. 17:21-27; Asano et al. (2004) “A Diabody For Cancer Immunotherapy And Its Functional Enhancement By Fusion Of Human Fc Domain,” Abstract 3P-683, J. Biochem. 76(8):992; Takemura, S. et al. (2000) “Construction Of A Diabody (Small Recombinant Bispecific Antibody) Using A Refolding System,” Protein Eng. 13(8):583-588; Lu, D. et al. (2005) “A Fully Human Recombinant IgG-Like Bispecific Antibody To Both The Epidermal Growth Factor Receptor And The Insulin-Like Growth Factor Receptor For Enhanced Antitumor Activity,” J. Biol. Chem. 280(20):19665-19672).

However, the art has recognized that bispecific diabodies composed of non-covalently associated polypeptides are unstable and readily dissociate into non-functional single polypeptide chain monomers (see, e.g., Lu, D. et al. (2005) “A Fully Human Recombinant IgG-Like Bispecific Antibody To Both The Epidermal Growth Factor Receptor And The Insulin-Like Growth Factor Receptor For Enhanced Antitumor Activity,” J. Biol. Chem. 280(20):19665-19672).

In the face of this challenge, the art has however succeeded in developing stable, covalently bonded heterodimeric non-mono-specific diabodies, termed DART® (Dual-Affinity Re-Targeting Reagents) diabodies, see, e.g., Chichili, G. R. et al. (2015) “A CD3×CD123 Bispecific DART For Redirecting Host T Cells To Myelogenous Leukemia: Preclinical Activity And Safety In Nonhuman Primates,” Sci. Transl. Med. 7(289):289ra82; Johnson, S. et al. (2010) “Effector Cell Recruitment With Novel Fv-Based Dual-Affinity Re-Targeting Protein Leads To Potent Tumor Cytolysis And In Vivo B-Cell Depletion,” J. Molec. Biol. 399(3):436-449; Veri, M. C. et al. (2010) “Therapeutic Control Of B Cell Activation Via Recruitment Of Fcgamma Receptor IIB (CD32B) Inhibitory Function With A Novel Bispecific Antibody Scaffold,” Arthritis Rheum. 62(7):1933-1943; Moore, P. A. et al. (2011) “Application Of Dual Affinity Retargeting Molecules To Achieve Optimal Redirected T cell Killing Of B-Cell Lymphoma,” Blood 117(17):4542-4551; U.S. Pat. Nos. 8,044,180; 8,133,982; 8,187,593; 8,193,318; 8,530,627; 8,669,349; 8,778,339; 8,784,808; 8,795,667; 8,802,091; 8,802,093; 8,946,387; 8,968,730; and 8,993,730; US Patent Publications No. 2009/0060910; 2010/0174053; 2011/0081347; 2011/0097323; 2011/0117089; 2012/0009186; 2012/0034221; 2012/0141476; 2012/0294796; 2013/0149236; 2013/0295121; 2014/0017237; and 2014/0099318; European Patent Documents No. EP 1868650; EP 2158221; EP 2247304; EP 2252631; EP 2282770; EP 2328934; EP 2376109; EP 2542256; EP 2601216; EP 2714079; EP 2714733; EP 2786762; EP 2839842; EP 2840091; and PCT Publications No. WO 2006/113665; WO 2008/157379; WO 2010/027797; WO 2010/033279; WO 2010/080538; WO 2011/109400; WO 2012/018687; WO 2012/162067; WO 2012/162068; WO 2014/159940; WO 2015/021089; WO 2015/026892; and WO 2015/026894). Such diabodies comprise two or more covalently complexed polypeptides and involve engineering one or more cysteine residues into each of the employed polypeptide species. For example, the addition of a cysteine residue to the C-terminus of such constructs has been shown to allow disulfide bonding between the polypeptide chains, stabilizing the resulting heterodimer without interfering with the binding characteristics of the bivalent molecule.

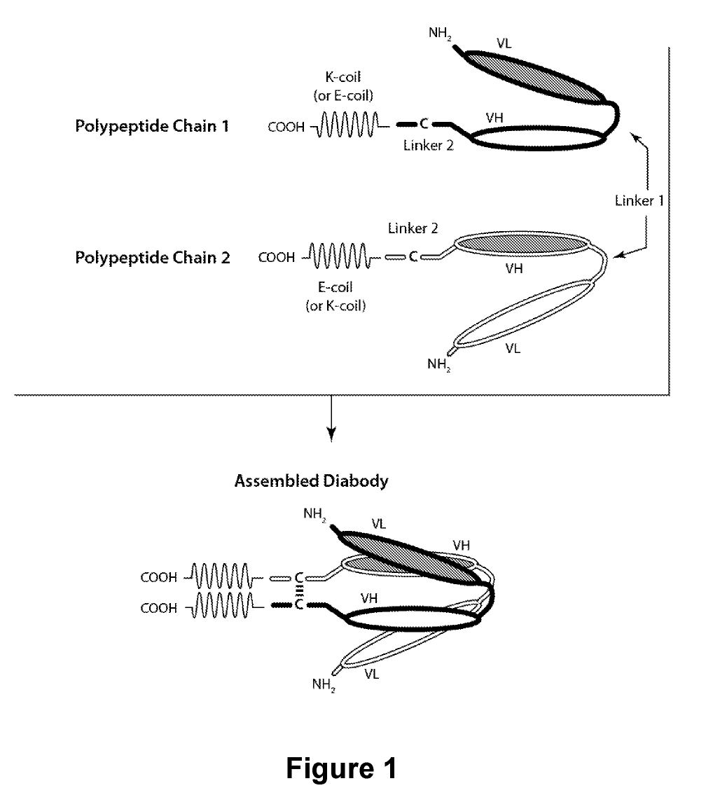

The simplest DART® diabody comprises two polypeptide chains each comprising three Domains (FIG. 1). The first polypeptide chain comprises: (i) a Domain that comprises a binding region of a light chain variable Domain of the a first immunoglobulin (VL1), (ii) a second Domain that comprises a binding region of a heavy chain variable Domain of a second immunoglobulin (VH2), and (iii) a third Domain that serves to promote heterodimerization (a “Heterodimer-Promoting Domain”) with the second polypeptide chain and to covalently bond the first polypeptide to the second polypeptide chain of the diabody. The second polypeptide chain contains a complementary first Domain (a VL2 Domain), a complementary second Domain (a VH1 Domain) and a third Domain that complexes with the third Domain of the first polypeptide chain in order to promote heterodimerization (a “Heterodimer-Promoting Domain”) and covalent bonding with the first polypeptide chain. Such molecules are stable, potent and have the ability to simultaneously bind two or more antigens. They are able to promote redirected T cell mediated killing of cells expressing target antigens. In one embodiment, the third Domains of the first and second polypeptide chains each contain a cysteine (“C”) residue, which serves to bind the polypeptides together via a disulfide bond. The third Domain of one or both of the polypeptide chains may additionally possesses the sequence of a CH2-CH3 Domain, such that complexing of the diabody polypeptides forms an Fc Domain that is capable of binding to the Fc receptor of cells (such as B lymphocytes, dendritic cells, natural killer cells, macrophages, neutrophils, eosinophils, basophils and mast cells). Many variations of such molecules have been described (see, e.g., United States Patent Publications No. 2013-0295121; 2010-0174053 and 2009-0060910; European Patent Publication No. EP 2714079; EP 2601216; EP 2376109; EP 2158221 and PCT Publications No. WO 2012/162068; WO 2012/018687; WO 2010/080538).

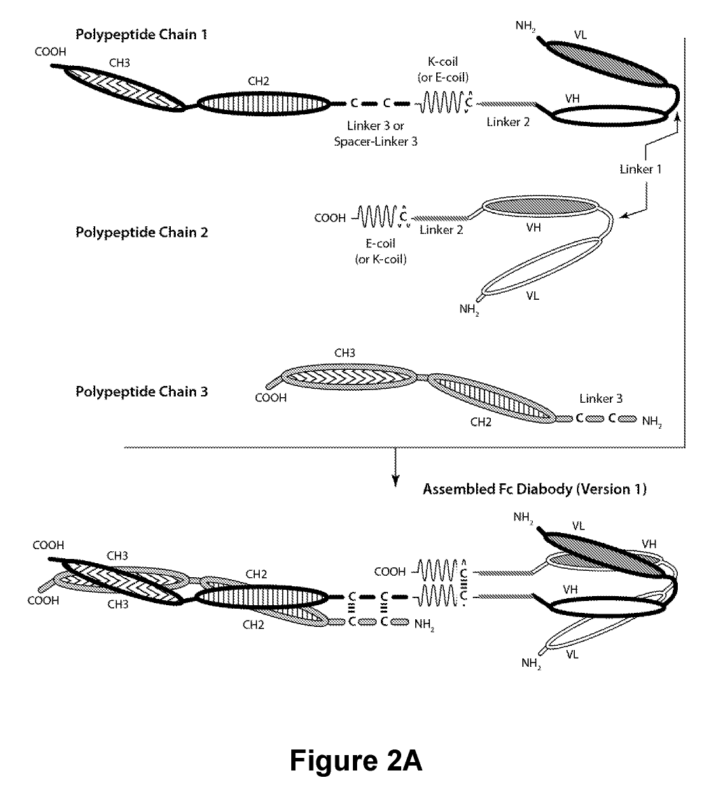

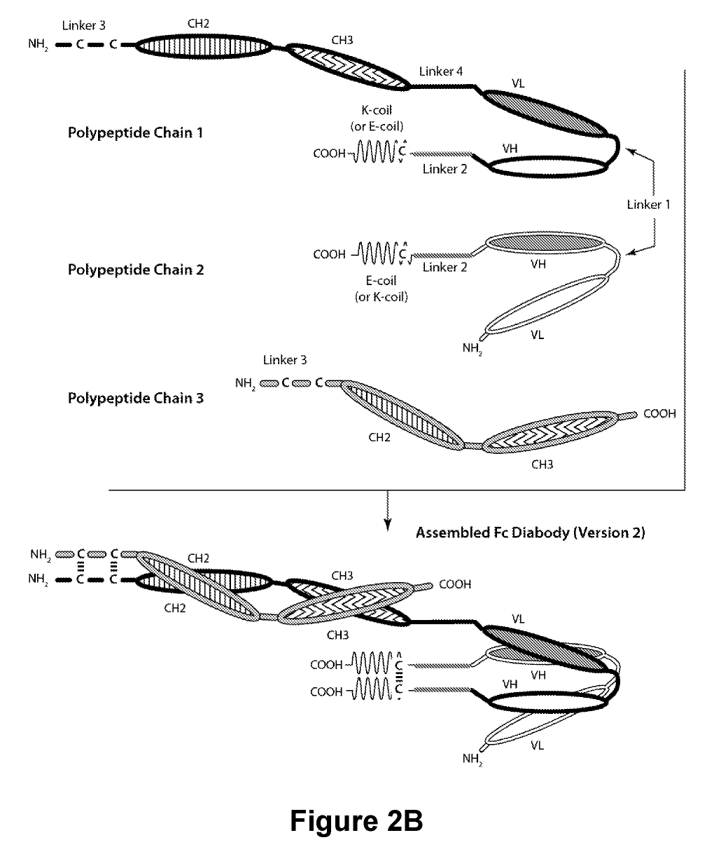

The preferred Fc-bearing DART® diabodies of the present invention comprise three polypeptide chains, and are depicted in FIGS. 2A-2B. The first polypeptide chain of such a diabody contains four Domains: (i) a VL1-containing Domain, (ii) a VH2-containing Domain, (iii) a Domain that promotes heterodimerization (a “Heterodimer-Promoting Domain”) and covalent bonding with the diabody's second polypeptide chain, and (iv) a Domain containing a CH2-CH3 sequence. The second polypeptide of such DART® diabodies contains: (i) a VL2-containing Domain, (ii) a VH1-containing Domain and (iii) a Domain that promotes heterodimerization (a “Heterodimer-Promoting Domain”) and covalent bonding with the diabody's first polypeptide chain. The third polypeptide of such DART® diabodies comprises a CH2-CH3 sequence. Thus, the first and second polypeptide chains of such DART® diabodies associate together to form a VL1/VH1 binding site that is capable of binding to a first epitope (1), as well as a VL2/VH2 binding site that is capable of binding to a second epitope (2). The preferred Fc-bearing DART® diabodies of the present invention are B7-H3×CD3 bispecific monovalent diabodies that are capable of binding to the “first epitope,” which may be either CD3 or B7-H3, and the “second epitope,” which is B7-H3 when the first epitope is CD3, and is CD3 when the first epitope is B7-H3. The first and second polypeptides are bonded to one another through one or more disulfide bonds involving cysteine residues in their respective linkers and/or third Domains. Notably, the first and third polypeptide chains complex with one another to form an Fc Domain that is stabilized via a disulfide bond. Such diabodies have enhanced potency. Preferred Fc-bearing DARTs® diabodies of the present invention may have either of two orientations (Table 1):

TABLE 1

| | | |
|---|---|---|
| First | 3^{rd} Chain | NH₂—CH2—CH3—COOH |
| Orientation | 1^{st} Chain | NH₂-VL1-VH2-Heterodimer-Promoting |
| | | Domain-CH2—CH3—COOH |
| | 2^{nd} Chain | NH₂-VL2-VH1-Heterodimer-Promoting |
| | | Domain-COOH |
| Second | 3^{rd} Chain | NH₂—CH2—CH3—COOH |
| Orientation | 1^{st} Chain | NH₂—CH2—CH3-VL1-VH2- |
| | | Heterodimer-Promoting Domain-COOH |
| | 2^{nd} Chain | NH₂-VL2-VH1-Heterodimer-Promoting |
| | | Domain-COOH |

### II. Preferred B7-H3×CD3 Bispecific Monovalent Diabodies of the Present Invention

The present invention is particularly directed to such Fc-bearing DARTs® diabodies that are capable of simultaneous binding to B7-H3 and CD3, and are thus B7-H3×CD3 bispecific monovalent DART® diabodies, and to the uses of such molecules in the treatment of cancer and other diseases and conditions. Although non-optimized B7-H3×CD3 bispecific monovalent diabodies are fully functional, analogous to the improvements obtained in gene expression through codon optimization (see, e.g., Grosjean, H. et al. (1982) “Preferential Codon Usage In Prokaryotic Genes: The Optimal Codon Anticodon Interaction Energy And The Selective Codon Usage In Efficiently Expressed Genes” Gene 18(3): 199-209), it is possible to further enhance the stability and/or function of B7-H3×CD3 bispecific monovalent diabodies by modifying or refining their sequences.

Preferably, as shown in FIG. 2A, the first of such three polypeptide chains will contain, in the N-terminal to C-terminal direction, an N-terminus, a Light Chain Variable Domain (VL) capable of binding to an epitope of a “first” antigen (VL1) (either CD3 or B7-H3), a Heavy Chain Variable Domain (VH) capable of binding to an epitope of a “second” antigen (VH2) (B7-H3, if the first antigen was CD3; CD3, if the first antigen was B7-H3), a Heterodimer-Promoting Domain, and a C-terminus. An intervening linker peptide (Linker 1) separates the Light Chain Variable Domain (VL1) from the Heavy Chain Variable Domain (VH2). Preferably, the Heavy Chain Variable Domain (VL2) is linked to a Heterodimer-Promoting Domain by an intervening linker peptide (Linker 2). In a preferred B7-H3×CD3 bispecific monovalent Fc diabody embodiment, the C-terminus of the Heterodimer-Promoting Domain is linked to the CH2-CH3 domains of an Fc Region (“Fc Domain”) by an intervening linker peptide (Linker 3) or by an intervening spacer-linker peptide (Spacer-Linker 3). Most preferably, the first of the three polypeptide chains will thus contain, in the N-terminal to C-terminal direction: VL1-Linker 1-VH2-Linker 2-Heterodimer-Promoting Domain-Spacer-Linker 3-Fc Domain.

Alternatively, as shown in FIG. 2B, the first of such three polypeptide chains will contain, in the N-terminal to C-terminal direction, an N-terminus, Linker 3, the CH2-CH3 domains of an Fc Region (“Fc Domain”), an intervening spacer peptide (Linker 4), having, for example the amino acid sequence: APSSS (SEQ ID NO:51) or the amino acid sequence APSSSPME (SEQ ID NO:52), a Light Chain Variable Domain (VL) capable of binding to an epitope of a “first” antigen (VL1) (either CD3 or B7-H3), a Heavy Chain Variable Domain (VII) capable of binding to an epitope of a “second” antigen (VH2) (B7-H3, if the first antigen was CD3; CD3, if the first antigen was B7-H3), a Heterodimer-Promoting Domain, and a C-terminus. An intervening linker peptide (Linker 1) separates the Light Chain Variable Domain (VL1) from the Heavy Chain Variable Domain (VH2). Preferably, the Heavy Chain Variable Domain (VH2) is linked to a Heterodimer-Promoting Domain by an intervening linker peptide (Linker 2). Most preferably, in such alternative orientation, the first of the three polypeptide chains will thus contain, in the N-terminal to C-terminal direction: Linker 3-Fc Domain-Linker 4-VL1-Linker 1-VH2-Linker 2-Heterodimer-Promoting Domain.

Preferably, for either of such two preferred orientations, the second of such three polypeptide chains will contain, in the N-terminal to C-terminal direction, an N-terminus, a Light Chain Variable Domain (VL) capable of binding to the epitope of the “second” antigen (VL2), a Heavy Chain Variable Domain (VII) capable of binding to the epitope of the “first” antigen (VH1), a Heterodimer-Promoting Domain and a C-terminus. An intervening linker peptide (Linker 1) separates the Light Chain Variable Domain (VL2) from the Heavy Chain Variable Domain (VH1). Preferably, the Heavy Chain Variable Domain (VH1) is linked to the Heterodimer-Promoting Domain by an intervening linker peptide (Linker 2). Most preferably, the second of the three polypeptide chains will thus contain, in the N-terminal to C-terminal direction: VL1-Linker 1-VH2-Linker 2-Heterodimer-Promoting Domain.

Preferably, for either of such two preferred orientations, the third of such three polypeptide chains will contain the linker peptide (Linker 3) and the CH2-CH3 domains of an Fc region (“Fc Domain”). As the third chain polypeptide chain does not comprise a VL Domain or a VH Domain the third polypeptide chain may be identical between two or more different B7-H3×CD3 bispecific monovalent Fc diabodies of the present invention.

The Light Chain Variable Domain of the first polypeptide chain (VL1) is coordinately selected so as to permit it to interact with the Heavy Chain Variable Domain of the second polypeptide chain (VH1) to thereby form a functional epitope-binding site that is capable of immunospecifically binding an epitope of the first antigen (i.e., either B7-H3 or CD3). Likewise, the Light Chain Variable Domain of the second polypeptide chain (VL2) is coordinately selected so as to permit it to interact with the Heavy Chain Variable Domain of the first polypeptide chain (VH2) to thereby form a functional epitope-binding site that is capable of immunospecifically binding an epitope of the second antigen (i.e., either B7-H3 or CD3). Thus, the selection of the Light Chain Variable Domains and the Heavy Chain Variable Domains are coordinated, such that the two polypeptide chains collectively comprise epitope-binding sites capable of binding to B7-H3 and CD3.

A. Preferred Linkers

Most preferably, the length of Linker 1, which separates such VL and VH domains of a polypeptide chain is selected to substantially or completely prevent such VL and VH domains from binding to one another (e.g., 12 or less amino acid residues in length). Thus the VL1 and VH2 domains of the first polypeptide chain are substantially or completely incapable of binding to one another, and do not form an epitope binding site that is capable of substantially binding to either the first or second antigen. Likewise, the VL2 and VH1 domains of the second polypeptide chain are substantially or completely incapable of binding to one another, and do not form an epitope binding site that is capable of substantially binding to either the first or second antigen. A preferred intervening spacer peptide (Linker 1) has the sequence (SEQ ID NO:1): GGGSGGGG.

The purpose of Linker 2 is to separate the VH Domain of a polypeptide chain from the optionally present Heterodimer-Promoting Domain of that polypeptide chain. Any of a variety of linkers can be used for the purpose of Linker 2. A preferred sequence for such Linker 2 has the amino acid sequence: GGCGGG (SEQ ID NO:2), which possesses a cysteine residue that may be used to covalently bond the first and second polypeptide chains to one another via a disulfide bond, or ASTKG (SEQ ID NO:3), which is derived from the IgG CH1 domain. Since the Linker 2, ASTKG (SEQ ID NO:3) does not possess such a cysteine, the use of such Linker 2 is preferably associated with the use of a cysteine-containing Heterodimer-Promoting Domain, such as the E-coil of SEQ ID NO:12 or the K-coil of SEQ ID NO:13 (see below).

The purpose of Linker 3 is to separate the Heterodimer-Promoting Domain of a polypeptide chain from the Fc Domain of that polypeptide chain. Any of a variety of linkers can be used for the purpose of Linker 3. A preferred sequence for such Linker 3 has the amino acid sequence: DKTHTCPPCP (SEQ ID NO:4). A preferred sequence for Spacer-Linker 3 has the amino acid sequence: GGGDKTHTCPPCP (SEQ ID NO:5).

B. Preferred Heterodimer-Promoting Domains

The formation of heterodimers of the first and second polypeptide chains can be driven by the inclusion of “Heterodimer-Promoting Domains.” Such domains include GVEPKSC (SEQ ID NO:6) or VEPKSC (SEQ ID NO:7) on one polypeptide chain and GFNRGEC (SEQ ID NO:8) or FNRGEC (SEQ ID NO:9) on the other polypeptide chain (US2007/0004909).

More preferably, however, the Heterodimer-Promoting Domains of the present invention are formed from one, two, three or four tandemly repeated coil domains of opposing charge that comprise a sequence of at least six, at least seven or at least eight charged amino acid residues (Apostolovic, B. et al. (2008) “pH-Sensitivity of the E3/K3 Heterodimeric Coiled Coil,” Biomacromolecules 9:3173-3180; Arndt, K. M. et al. (2001) “Helix-stabilized Fv (hsFv) Antibody Fragments: Substituting the Constant Domains of a Fab Fragment for a Heterodimeric Coiled-coil Domain,” J. Molec. Biol. 312:221-228; Arndt, K. M. et al. (2002) “Comparison of In Vivo Selection and Rational Design of Heterodimeric Coiled Coils,” Structure 10:1235-1248; Boucher, C. et al. (2010) “Protein Detection By Western Blot Via Coiled—Coil Interactions,” Analytical Biochemistry 399:138-140; Cachia, P. J. et al. (2004) “Synthetic Peptide Vaccine Development: Measurement Of Polyclonal Antibody Affinity And Cross Reactivity Using A New Peptide Capture And Release System For Surface Plasmon Resonance Spectroscopy,” J. Mol. Recognit. 17:540-557; De Crescenzo, G. D. et al. (2003) “Real-Time Monitoring of the Interactions of Two-Stranded de novo Designed Coiled-Coils: Effect of Chain Length on the Kinetic and Thermodynamic Constants of Binding,” Biochemistry 42:1754-1763; Fernandez-Rodriquez, J. et al. (2012) “Induced Heterodimerization And Purification Of Two Target Proteins By A Synthetic Coiled-Coil Tag,” Protein Science 21:511-519; Ghosh, T. S. et al. (2009) “End-To-End And End-To-Middle Interhelical Interactions: New Classes Of Interacting Helix Pairs In Protein Structures,” Acta Crystallographica D65:1032-1041; Grigoryan, G. et al. (2008) “Structural Specificity In Coiled-Coil Interactions,” Curr. Opin. Struc. Biol. 18:477-483; Litowski, J. R. et al. (2002) “Designing Heterodimeric Two-Stranded a-Helical Coiled-Coils: The Effects Of Hydrophobicity And a-Helical Propensity On Protein Folding, Stability, And Specificity,” J. Biol. Chem. 277:37272-37279; Steinkruger, J. D. et al. (2012) “The d′-d-d′ Vertical Triad is Less Discriminating Than the a′-a-a′ Vertical Triad in the Antiparallel Coiled-coil Dimer Motif,” J. Amer. Chem. Soc. 134(5):2626-2633; Straussman, R. et al. (2007) “Kinking the Coiled Coil-Negatively Charged Residues at the Coiled-coil Interface,” J. Molec. Biol. 366:1232-1242; Tripet, B. et al. (2002) “Kinetic Analysis of the Interactions between Troponin C and the C-terminal Troponin I Regulatory Region and Validation of a New Peptide Delivery/Capture System used for Surface Plasmon Resonance,” J. Molec. Biol. 323:345-362; Woolfson, D. N. (2005) “The Design Of Coiled-Coil Structures And Assemblies,” Adv. Prot. Chem. 70:79-112; Zeng, Y. et al. (2008) “A Ligand-Pseudoreceptor System Based On de novo Designed Peptides For The Generation Of Adenoviral Vectors With Altered Tropism,” J. Gene Med. 10:355-367).

Such repeated coil domains may be exact repeats or may have substitutions. For example, the Heterodimer-Promoting Domain of one polypeptide chain may comprise a sequence of negatively charged amino acid residues and the Heterodimer-Promoting Domain of the other polypeptide chain may comprise a sequence of negatively charged amino acid residues. In particular, embodiments the coil domains comprise eight negatively charged amino acid residues or eight positively charged residues. It is immaterial which coil is provided to the first or second polypeptide chains, provided that a coil of opposite charge is used for the other polypeptide chain. However, a preferred B7-H3×CD3 bispecific monovalent Fc diabody of the present invention has a first polypeptide chain having a negatively charged coil. The positively charged amino acid of a positively charged coil domain may be lysine, arginine, histidine, etc., and is preferably lysine. The negatively charged amino acid of a negatively charged coil may be glutamic acid, aspartic acid, etc., and is preferably glutamic acid.

The B7-H3×CD3 bispecific monovalent DART® diabodies of the present invention may possess only a single Heterodimer-Promoting Domain (i.e., either the first polypeptide chain or the second polypeptide chain, but not both, will contain a Heterodimer-Promoting Domain. The presence of such single Heterodimer-Promoting Domain promotes heterodimerization by impeding the formation of diabodies that are homodimers (such molecules either lacking any Heterodimer-Promoting Domain, or possessing two repelling (like-charged) Heterodimer-Promoting Domains). However, it is preferred for both the first and second polypeptide chains of the diabodies of the present invention to contain Heterodimer-Promoting Domains.

In a preferred embodiment, one of the Heterodimer-Promoting Domains will comprise four tandem “E-coil” helical domains (SEQ ID NO:10: EVAALEK-EVAALEK-EVAALEK-EVAALEK), whose glutamate residues will form a negative charge at pH 7, while the other of the Heterodimer-Promoting Domains will comprise four tandem “K-coil” domains (SEQ ID NO:11: KVAALKE-KVAALKE-KVAALKE-KVAALKE), whose lysine residues will form a positive charge at pH 7. The presence of such charged domains promotes association between the first and second polypeptides, and thus fosters heterodimerization. In another preferred embodiment a Heterodimer-Promoting Domain in which one of the four tandem “E-coil” helical domains of SEQ ID NO:10 has been modified to contain a cysteine residue: EVAA [symbol] K-EVAALEK-EVAALEK-EVAALEK (SEQ ID NO:12) is utilized. Likewise, in another preferred embodiment, a Heterodimer-Promoting Domain in which one of the four tandem “K-coil” helical domains of SEQ ID NO:11 has been modified to contain a cysteine residue: KVAA [symbol] E-KVAALKE-KVAALKE-KVAALKE (SEQ ID NO:13) is utilized.

C. Covalent Bonding of the Polypeptide Chains

The B7-H3×CD3 bispecific monovalent Fc diabodies of the present invention are engineered so that their first and second polypeptide chains covalently bond to one another via one or more cysteine residues positioned along their length. Such cysteine residues may be introduced into the intervening linker that separates the VL and VH domains of the polypeptides. Alternatively, Linker 2 may contain a cysteine residue. Additionally, or alternatively, Linker 3 may contain a cysteine residue, as in SEQ ID NO:4 or SEQ ID NO:5. Most preferably, one or more coil domains of the Heterodimer-Promoting Domain will be substituted to contain a cysteine residue as in SEQ ID NO:12 or SEQ ID NO:13.

D. Preferred Fc Domains

The Fc Domain of the preferred B7-H3×CD3 bispecific monovalent Fc diabodies of the present invention may be either a complete Fc region (e.g., a complete IgG Fc region) or only a fragment of a complete Fc region. Although the Fc Domain of the preferred bispecific monovalent Fc diabodies of the present invention may possess the ability to bind to one or more Fc receptors (e.g., FcγR(s)), more preferably such Fc Domain will have been modified to cause reduced binding to FcγRIA (CD64), FcγRIIA (CD32A), FcγRIIB (CD32B), FcγRIIIA (CD16a) or FcγRIBB (CD16b) (relative to the binding exhibited by a wild-type Fc region) or will have been modified to have substantially eliminated the ability of such Fc Domain to bind to such receptor(s). The Fc Domain of the preferred bispecific monovalent Fc diabodies of the present invention may thus include some or all of the CH2 Domain and/or some or all of the CH3 Domain of a complete Fc region, or may comprise a variant CH2 and/or a variant CH3 sequence (that may include, for example, one or more insertions and/or one or more deletions with respect to the CH2 or CH3 domains of a complete Fc region). The Fc Domain of the bispecific monovalent Fc diabodies of the present invention may comprise non-Fc polypeptide portions, or may comprise portions of non-naturally complete Fc regions, or may comprise non-naturally occurring orientations of CH2 and/or CH3 domains (such as, for example, two CH2 domains or two CH3 domains, or in the N-terminal to C-terminal direction, a CH3 Domain linked to a CH2 Domain, etc.).

In a preferred embodiment the first and third polypeptide chains of the B7-H3×CD3 bispecific monovalent Fc diabodies of the present invention each comprise CH2-CH3 domains that complex together to form an immunoglobulin (IgG) Fc Domain. The amino acid sequence of an exemplary CH2-CH3 domain of human IgG1 is (SEQ ID NO:14):

```
231 240 250 260 270
APELLGGPSV FLFPPKPKDT LMISRTPEVT CVVVDVSHED
              280 290 300 310
PEVKFNWYVD GVEVHNAKTK PREEQYNSTY RVVSVLTVLH
              320 330 340 350
QDWLNGKEYK CKVSNKALPA PIEKTISKAK GQPREPQVYT
              360 370 380 390
LPPSREEMTK NQVSLTCLVK GFYPSDIAVE WESNGQPENN
              400 410 420 430
YKTTPPVLDS DGSFFLYSKL TVDKSRWQQG NVFSCSVMHE
              440 447
ALHNHYTQKS LSLSPGK
```

The numbering of the residues in the constant regions of an IgG heavy chain is that of the EU index as in Kabat et al., Sequences of Proteins of Immunological Interest, 5^{th} Ed. Public Health Service, NH1, MD (1991), expressly incorporated herein by references. The “EU index as in Kabat” refers to the numbering of the human IgG1 EU antibody. Polymorphisms have been observed at a number of different positions within antibody constant regions (e.g., Fc positions, including but not limited to positions 270, 272, 312, 315, 356, and 358 as numbered by the EU index as set forth in Kabat), and thus slight differences between the presented sequence and sequences in the prior art can exist. Polymorphic forms of human immunoglobulins have been well-characterized. At present, 18 Gm allotypes are known: G1m (1, 2, 3, 17) or G1m (a, x, f, z), G2m (23) or G2m (n), G3m (5, 6, 10, 11, 13, 14, 15, 16, 21, 24, 26, 27, 28) or G3m (b1, c3, b3, b0, b3, b4, s, t, g1, c5, u, v, g5) (Lefranc, et al., “The Human IgG Subclasses: Molecular Analysis Of Structure, Function And Regulation.” Pergamon, Oxford, pp. 43-78 (1990); Lefranc, G. et al., 1979, Hum. Genet.: 50, 199-211). It is specifically contemplated that the B7-H3×CD3 bispecific monovalent Fc diabodies of the present invention may be incorporate any allotype, isoallotype, or haplotype of any immunoglobulin gene, and are not limited to the allotype, isoallotype or haplotype of the sequences provided herein. Furthermore, in some expression systems the C-terminal amino acid residue (bolded above) of the CH3 Domain may be post-translationally removed. Accordingly, the C-terminal residue of the CH3 Domain is an optional amino acid residue in the B7-H3×CD3 bispecific monovalent Fc diabodies of the invention. Exemplary B7-H3×CD3 bispecific monovalent Fc diabodies comprising the C-terminal residue of SEQ ID NO:14 are provided below. Also specifically encompassed by the instant invention are such constructs that lack the C-terminal lysine residue of SEQ ID NO:14.

The CH2 and/or CH3 Domains of the first and third polypeptide chains may both be composed of SEQ ID NO:14, or a variant thereof.

In particular, it is preferred for the CH2-CH3 domains of the first and third polypeptide chains of the B7-H3×CD3 bispecific monovalent Fc diabodies of the present invention to exhibit decreased (or substantially no) binding to FcγRIA (CD64), FcγRIIA (CD32A), FcγRIIB (CD32B), FcγRIIIA (CD16a) or FcγRIIIB (CD16b) (relative to the binding exhibited by the wild-type Fc region (SEQ ID NO:14)). Fc variants and mutant forms capable of mediating such altered binding are well known in the art and include amino acid substitutions at positions 234 and 235, a substitution at position 265 or a substitution at position 297, wherein said numbering is that of the EU index as in Kabat (see, for example, U.S. Pat. No. 5,624,821, herein incorporated by reference). In a preferred embodiment the CH2-CH3 Domain of the first and/or third polypeptide chains of the B7-H3×CD3 bispecific monovalent Fc diabodies of the present invention include a substitution at position 234 with alanine and 235 with alanine, wherein said numbering is that of the EU index as in Kabat.

The CH2 and/or CH3 Domains of the first and third polypeptide chains need not be identical in sequence, and advantageously are modified to foster complexing between the two polypeptide chains. For example, an amino acid substitution (preferably a substitution with an amino acid comprising a bulky side group forming a “knob,” e.g., tryptophan) can be introduced into the CH2 or CH3 Domain such that steric interference will prevent interaction with a similarly mutated domain and will obligate the mutated domain to pair with a domain into which a complementary, or accommodating mutation has been engineered, i.e., a “hole” (e.g., a substitution with glycine). Such sets of mutations can be engineered into any pair of polypeptides comprising the bispecific monovalent Fc diabody molecule, and further, engineered into any portion of the polypeptides chains of said pair. Methods of protein engineering to favor heterodimerization over homodimerization are well known in the art, in particular with respect to the engineering of immunoglobulin-like molecules, and are encompassed herein (see e.g., Ridgway et al. (1996) “‘Knobs-Into-Holes’ Engineering Of Antibody CH3 Domains For Heavy Chain Heterodimerization,” Protein Engr. 9:617-621, Atwell et al. (1997) “Stable Heterodimers From Remodeling The Domain Interface Of A Homodimer Using A Phage Display Library,” J. Mol. Biol. 270: 26-35, and Xie et al. (2005) “A New Format Of Bispecific Antibody: Highly Efficient Heterodimerization, Expression And Tumor Cell Lysis,” J. Immunol. Methods 296:95-101; each of which documents is hereby incorporated herein by reference in its entirety). Preferably the knob is engineered into the CH2-CH3 Domains of the first polypeptide chain and the hole is engineered into the CH2-CH3 Domains of the third polypeptide chain. Thus, the knob will help in preventing two molecules of the first polypeptide chain from homodimerizing via their CH2 and/or CH3 Domains. As the third polypeptide chain preferably contains the hole substitution it will have the ability to heterodimerize with the first polypeptide chain as well as homodimerize with itself (however, such homodimerization does not form a molecule possessing epitope-binding sites). A preferred knob is created by modifying a native IgG Fc Domain to contain the modification T366W. A preferred hole is created by modifying a native IgG Fc Domain to contain the modification T366S, L368A and Y407V. To aid in purifying the third polypeptide chain homodimer from the final bispecific monovalent Fc diabody comprising the first, second and third polypeptide chains, the protein A binding site of the CH2 and CH3 Domains of the third polypeptide chain is preferably mutated by amino acid substitution at position 435 (H435R). Thus, the third polypeptide chain homodimer will not bind to protein A, whereas the bispecific monovalent Fc diabody will retain its ability to bind protein A via the protein A binding site on the first polypeptide chain.

A preferred sequence for the CH2 and CH3 Domains of the first polypeptide chain of the B7-H3×CD3 bispecific monovalent Fc diabodies of the present invention will have the “knob-bearing” sequence (SEQ ID NO:15):

APEAAGGPSV FLFPPKPKDT LMISRTPEVT CVVVDVSHED
PEVKFNWYVD GVEVHNAKTK PREEQYNSTY RVVSVLTVLH
QDWLNGKEYK CKVSNKALPA PIEKTISKAK GQPREPQVYT
LPPSREEMTK NQVSLWCLVK GFYPSDIAVE WESNGQPENN
YKTTPPVLDS DGSFFLYSKL TVDKSRWQQG NVFSCSVMHE
ALHNHYTQKS LSLSPGK

A preferred sequence for the CH2 and CH3 Domains of the third polypeptide chain of the B7-H3×CD3 bispecific monovalent Fc diabodies of the present invention will have the “hole-bearing” sequence (SEQ ID NO:16):

APEAAGGPSV FLFPPKPKDT LMISRTPEVT CVVVDVSHED
PEVKFNWYVD GVEVHNAKTK PREEQYNSTY RVVSVLTVLH
QDWLNGKEYK CKVSNKALPA PIEKTISKAK GQPREPQVYT
LPPSREEMTK NQVSLSCAVK GFYPSDIAVE WESNGQPENN
YKTTPPVLDS DGSFFLVSKL TVDKSRWQQG NVFSCSVMHE
ALHNRYTQKS LSLSPGK

As will be noted, the CH2-CH3 Domains of SEQ ID NO:15 and SEQ ID NO:16 include a substitution at position 234 with alanine and 235 with alanine, and thus form an Fc Domain that exhibits decreased (or substantially no) binding to FcγRIA (CD64), FcγRIIA (CD32A), FcγRIIB (CD32B), FcγRIIIA (CD16a) or FcγRIIIB (CD16b) (relative to the binding exhibited by the wild-type Fc region (SEQ ID NO:14). Furthermore, specifically encompassed by the instant invention are B7-H3×CD3 bispecific monovalent Fc diabodies constructs lacking the C-terminal lysine residue of SEQ ID NO:14, SEQ ID NO:15, and/or SEQ ID NO:16.

It is preferred that the first polypeptide chain will have a “knob-bearing” CH2-CH3 sequence, such as that of SEQ ID NO:15. However, as will be recognized, a “hole-bearing” CH2-CH3 Domain (e.g., SEQ ID NO:16) could be employed in the first polypeptide chain, in which case, a “knob-bearing” CH2-CH3 Domain (e.g., SEQ ID NO:15) would be employed in the third polypeptide chain.

E. Preferred B7-H3 Variable Domains

The Antigen-Binding Domain of any anti-B7-H3 antibody may be used in accordance with the present invention. Exemplary antibodies that are immunospecific for human B7-H7 (designated “B7-H3 mAb A,” “B7-H3 mAb B,” and “B7-H3 mAb C”) are provided below.

1. B7-H3 mAb A

The amino acid sequence of the VL Domain of B7-H3 mAb A (SEQ ID NO:17) is shown below (CDR_{L} residues are shown underlined):

DIQLTQSPSF LSASVGDRVT ITCKASQNVD TNVAWYQQKP
GKAPKALIYS ASYRYSGVPS RFSGSGSGTD FTLTISSLQP
EDFATYYCQQ YNNYPFTFGQ GTKLEIK
CDR_{L}1 of B7-H3 mAb A:

(SEQ ID NO: 18)

KASQNVDTNVA
CDR_{L}2 of B7-H3 mAb A:

(SEQ ID NO: 19)

SASYRYS
CDR_{L}3 of B7-H3 mAb A:

(SEQ ID NO: 20)

QQYNNYPFT

The amino acid sequence of the VH Domain of B7-H3 mAb A (SEQ ID NO:21) is shown below (CDR_{H} residues are shown underlined):

EVQLVESGGG LVQPGGSLRL SCAASGFTFS SFGMHWVRQA
PGKGLEWVAY ISSDSSAIYY ADTVKGRFTI SRDNAKNSLY
LQMNSLRDED TAVYYCGRGR ENIYYGSRLD YWGQGTTVTV SS
CDR_{H}1 of B7-H3 mAb A:

(SEQ ID NO: 22)

SFGMH
CDR_{H}2 of B7-H3 mAb A:

(SEQ ID NO: 23)

YISSDSSAIYYADTVKG
CDR_{H}3 of B7-H3 mAb A:

(SEQ ID NO: 24)

GRENIYYGSRLDY

2. B7-H3 mAb B

The amino acid sequence of the VL Domain of B7-H3 mAb B (SEQ ID NO:25) is shown below (CDR_{H} residues are shown underlined):

DIQMTQSPSS LSASVGDRVT ITCRASQDIS NYLNWYQQKP
GKAPKLLIYY TSRLHSGVPS RFSGSGSGTD FTLTISSLQP
EDIATYYCQQ GNTLPPTFGG GTKLEIK
CDR_{L}1 of B7-H3 mAb B:

(SEQ ID NO: 26)

RASQDISNYLN
CDR_{L}2 of B7-H3 mAb B:

(SEQ ID NO: 27)

YTSRLHS
CDR_{L}3 of B7-H3 mAb B:

(SEQ ID NO: 28)

QQGNTLPPT

The amino acid sequence of the VH Domain of B7-H3 mAb B (SEQ ID NO:29) is shown below (CDR_{H} residues are shown underlined):

QVQLVQSGAE VKKPGASVKV SCKASGYTFT SYWMQWVRQA
PGQGLEWMGT IYPGDGDTRY TQKFKGRVTI TADKSTSTAY
MELSSLRSED TAVYYCARRG IPRLWYFDVW GQGTTVTVSS
CDR_{H}1 of B7-H3 mAb B:

(SEQ ID NO: 30)

SYWMQ
CDR_{H}2 of B7-H3 mAb B:

(SEQ ID NO: 31)

TIYPGDGDTRYTQKFKG
CDR_{H}3 of B7-H3 mAb B:

(SEQ ID NO: 32)

RGIPRLWYFDV

3. B7-H3 mAb C

The amino acid sequence of the VL Domain of B7-H3 mAb C (SEQ ID NO:33) is shown below (CDR_{L} residues are shown underlined):

DIQMTQSPSS LSASVGDRVT ITCRASQSIS SYLNWYQQKP
GKAPKLLIYY TSRLQSGVPS RFSGSGSGTD FTLTISSLQP
EDIATYYCQQ GNTLPPTFGG GTKLEIK
CDR_{L}1 of B7-H3 mAb C:

(SEQ ID NO: 34)

RASQSISSYLN
CDR_{L}2 of B7-H3 mAb C:

(SEQ ID NO: 35)

YTSRLQS
CDR_{L}3 of B7-H3 mAb C:

(SEQ ID NO: 36)

QQGNTLPPT

The amino acid sequence of the VH Domain of B7-H3 mAb C (SEQ ID NO:37) is shown below (CDR_{H} residues are shown underlined):

QVQLVQSGAE VKKPGASVKV SCKASGYTFT SYWMQWVRQA
PGQGLEWMGT IYPGGGDTRY TQKFQGRVTI TADKSTSTAY
MELSSLRSED TAVYYCARRG IPRLWYFDVW GQGTTVTVSS
CDR_{H}1 of B7-H3 mAb C:

(SEQ ID NO: 38)

SYWMQ
CDR_{H}2 of B7-H3 mAb C:

(SEQ ID NO: 39)

TIYPGGGDTRYTQKFQG
CDR_{H}3 of B7-H3 mAb C:

(SEQ ID NO: 40)

RGIPRLWYFDV

F. Preferred CD3 Variable Domains

The Antigen-Binding Domain of any anti-CD3 antibody may be used in accordance with the present invention. An exemplary antibody that is immunospecific for human CD3 (designated “CD3 mAb A) is provided below.

The amino acid sequence of the VL Domain of CD3 mAb A (SEQ ID NO:41) is shown below (CDR_{L} residues are shown underlined):

QAVVTQEPSL TVSPGGTVTL TCRSSTGAVT TSNYANWVQQ
KPGQAPRGLI GGTNKRAPWT PARFSGSLLG GKAALTITGA
QAEDEADYYC ALWYSNLWVF GGGTKLTVLG
CDR_{L}1 of CD3 mAb A:

(SEQ ID NO: 42)

RSSTGAVTTSNYAN
CDR_{L}2 of CD3 mAb A:

(SEQ ID NO: 43)

GTNKRAP
CDR_{L}3 of CD3 mAb A:

(SEQ ID NO: 44)

ALWYSNLWV

The amino acid sequence of the VH Domain of CD3 mAb A (SEQ ID NO:45) is shown below (CDR_{H} residues are shown underlined):

EVQLVESGGG LVQPGGSLRL SCAASGFTFS TYAMNWVRQA
PGKGLEWVGR IRSKYNNYAT YYADSVKDRF TISRDDSKNS
LYLQMNSLKT EDTAVYYCVR HGNFGNSYVS WFAYWGQGTL VTVSS
CDR_{H}1 of CD3 mAb A:

(SEQ ID NO: 46)

TYAMN
CDR_{H}2 of CD3 mAb A:

(SEQ ID NO: 47)

RIRSKYNNYATYYADSVKD
CDR_{H}3 of CD3 mAb A:

(SEQ ID NO: 48)

HGNFGNSYVSWFAY

In certain embodiments, the VH Domain of CD3 mAb A comprises an aspartate to glycine substitution at Kabat position 65 (D65G substitution, corresponds to residue 68 of SEQ ID NO:45), such that the amino acid sequence of CDR_{H}2 is: RIRSKYNNYATYYADSVKG (SEQ ID NO:49). The amino acid sequence of the VH Domain of CD3 mAb A having the D65G substitution (SEQ ID NO:50) is shown below (the substituted residue is shown underlined):

EVQLVESGGG LVQPGGSLRL SCAASGFTFS TYAMNWVRQA
PGKGLEWVGR IRSKYNNYAT YYADSVKGRF TISRDDSKNS
LYLQMNSLKT EDTAVYYCVR HGNFGNSYVS WFAYWGQGTL VTVSS

### III. Exemplary B7-H3×CD3 Bispecific Monovalent Fc Diabodies

The invention provides B7-H3×CD3 bispecific monovalent Fc diabodies capable of simultaneously and specifically binding to B7-H3 and to CD3. As indicated above, the B7-H3×CD3 bispecific monovalent Fc diabodies of the present invention comprise three polypeptide chains. The polypeptide chains of four exemplary B7-H3×CD3 bispecific monovalent Fc diabodies capable that binding to B7-H3 and to CD3 (designated “DART-A,” “DART-B,” “DART-C,” and “DART-D”) are provided below.

A. DART-A

The first polypeptide chain of DART-A comprises, in the N-terminal to C-terminal direction, an N-terminus, a VL domain of a monoclonal antibody capable of binding to B7-H3 (VL_{B7-H3} B7-H3 mAb A) (SEQ ID NO:17), an intervening linker peptide (Linker 1; GGGSGGGG (SEQ ID NO:1)), a VH domain of a monoclonal antibody capable of binding to CD3 (VH_{CD3} CD3 mAb A) (SEQ ID NO:45), an intervening linker peptide (Linker 2; GGCGGG (SEQ ID NO:2)), a Heterodimer-Promoting (E-coil) Domain (EVAALEK-EVAALEK-EVAALEK-EVAALEK (SEQ ID NO:10)), an intervening linker peptide (Spacer-Linker 3; GGGDKTHTCPPCP (SEQ ID NO:5)), a “knob-bearing” Fc Domain (SEQ ID NO:15), and a C-terminus.

Thus, the first polypeptide chain of DART-A is composed of: SEQ ID NO:17-SEQ ID NO:1-SEQ ID NO:45-SEQ ID NO:2-SEQ ID NO:10-SEQ ID NO:5-SEQ ID NO:15.

The amino acid sequence of the first polypeptide of DART-A is (SEQ ID NO:53):

DIQLTQSPSF LSASVGDRVT ITCKASQNVD TNVAWYQQKP
GKAPKALIYS ASYRYSGVPS RFSGSGSGTD FTLTISSLQP
EDFATYYCQQ YNNYPFTFGQ GTKLEIKGGG SGGGGEVQLV
ESGGGLVQPG GSLRLSCAAS GFTFSTYAMN WVRQAPGKGL
EWVGRIRSKY NNYATYYADS VKDRFTISRD DSKNSLYLQM
NSLKTEDTAV YYCVRHGNFG NSYVSWFAYW GQGTLVTVSS
GGCGGGEVAA LEKEVAALEK EVAALEKEVA ALEKGGGDKT
HTCPPCPAPE AAGGPSVFLF PPKPKDTLMI SRTPEVTCVV
VDVSHEDPEV KFNWYVDGVE VHNAKTKPRE EQYNSTYRVV
SVLTVLHQDW LNGKEYKCKV SNKALPAPIE KTISKAKGQP
REPQVYTLPP SREEMTKNQV SLWCLVKGFY PSDIAVEWES
NGQPENNYKT TPPVLDSDGS FFLYSKLTVD KSRWQQGNVF
SCSVMHEALH NHYTQKSLSL SPGK

An exemplary polynucleotide encoding such a polypeptide is (SEQ ID NO:54):

gacatccagc tgacccagtc cccctccttc ctgtctgcct
ccgtgggcga cagagtgacc atcacatgca aggcctccca
gaacgtggac accaacgtgg cctggtatca gcagaagcct
ggcaaggccc ctaaggcgct gatctactcc gcctcctacc
ggtactccgg cgtgccttcc aggttctccg gctccggctc
tggcaccgac ttcaccctga ccatctccag cctgcagcct
gaggacttcg ccacctacta ctgccagcag tacaacaact
accctttcac cttcggccag ggcaccaagc tggaaatcaa
gggaggcgga tccggcggcg gaggcgaggt gcagctggtg
gagtctgggg gaggcttggt ccagcctgga gggtccctga
gactctcctg tgcagcctct ggattcacct tcagcacata
cgctatgaat tgggtccgcc aggctccagg gaaggggctg
gagtgggttg gaaggatcag gtccaagtac aacaattatg
caacctacta tgccgactct gtgaaggata gattcaccat
ctcaagagat gattcaaaga actcactgta tctgcaaatg
aacagcctga aaaccgagga cacggccgtg tattactgtg
tgagacacgg taacttcggc aattcttacg tgtcttggtt
tgcttattgg ggacagggga cactggtgac tgtgtcttcc
ggaggatgtg gcggtggaga agtggccgca ctggagaaag
aggttgctgc tttggagaag gaggtcgctg cacttgaaaa
ggaggtcgca gccctggaga aaggcggcgg ggacaaaact
cacacatgcc caccgtgccc agcacctgaa gccgcggggg
gaccgtcagt cttcctcttc cccccaaaac ccaaggacac
cctcatgatc tcccggaccc ctgaggtcac atgcgtggtg
gtggacgtga gccacgaaga ccctgaggtc aagttcaact
ggtacgtgga cggcgtggag gtgcataatg ccaagacaaa
gccgcgggag gagcagtaca acagcacgta ccgtgtggtc
agcgtcctca ccgtcctgca ccaggactgg ctgaatggca
aggagtacaa gtgcaaggtc tccaacaaag ccctcccagc
ccccatcgag aaaaccatct ccaaagccaa agggcagccc
cgagaaccac aggtgtacac cctgccccca tcccgggagg
agatgaccaa gaaccaggtc agcctgtggt gcctggtcaa
aggcttctat cccagcgaca tcgccgtgga gtgggagagc
aatgggcagc cggagaacaa ctacaagacc acgcctcccg
tgctggactc cgacggctcc ttcttcctct acagcaagct
caccgtggac aagagcaggt ggcagcaggg gaacgtcttc
tcatgctccg tgatgcatga ggctctgcac aaccactaca
cgcagaagag cctctccctg tctccgggta aa

The second polypeptide chain of DART-A comprises, in the N-terminal to C-terminal direction, an N-terminus, a VL domain of a monoclonal antibody capable of binding to CD3 (VL_{CD3} CD3 mAb A) (SEQ ID NO:41), an intervening linker peptide (Linker 1; GGGSGGGG (SEQ ID NO:1)), a VH domain of a monoclonal antibody capable of binding to B7-H3 (VH_{B7-H3} B7-H3 mAb A) (SEQ ID NO:21), an intervening linker peptide (Linker 2; GGCGGG (SEQ ID NO:2)), a Heterodimer-Promoting (K-coil) Domain (KVAALKE-KVAALKE-KVAALKE-KVAALKE (SEQ ID NO:11), and a C-terminus.

Thus, the second polypeptide of DART-A is composed of: SEQ ID NO:41-SEQ ID NO:1-SEQ ID NO:21-SEQ ID NO:2-SEQ ID NO:11.

The amino acid sequence of the second polypeptide of DART-A is (SEQ ID NO:55):

QAVVTQEPSL TVSPGGTVTL TCRSSTGAVT TSNYANWVQQ
KPGQAPRGLI GGTNKRAPWT PARFSGSLLG GKAALTITGA
QAEDEADYYC ALWYSNLWVF GGGTKLTVLG GGGSGGGGEV
QLVESGGGLV QPGGSLRLSC AASGFTFSSF GMHWVRQAPG
KGLEWVAYIS SDSSAIYYAD TVKGRFTISR DNAKNSLYLQ
MNSLRDEDTA VYYCGRGREN IYYGSRLDYW GQGTTVTVSS
GGCGGGKVAA LKEKVAALKE KVAALKEKVA ALKE

An exemplary polynucleotide encoding such a polypeptide has the sequence (SEQ ID NO:561:

caggctgtgg tgactcagga gccttcactg accgtgtccc
caggcggaac tgtgaccctg acatgcagat ccagcacagg
cgcagtgacc acatctaact acgccaattg ggtgcagcag
aagccaggac aggcaccaag gggcctgatc gggggtacaa
acaaaagggc tccctggacc cctgcacggt tttctggaag
tctgctgggc ggaaaggccg ctctgactat taccggggca
caggccgagg acgaagccga ttactattgt gctctgtggt
atagcaatct gtgggtgttc gggggtggca caaaactgac
tgtgctggga gggggtggat ccggcggcgg aggcgaggtg
cagctggtcg agtctggcgg aggactggtg cagcctggcg
gctccctgag actgtcttgc gccgcctccg gcttcacctt
ctccagcttc ggcatgcact gggtccgcca ggctccaggc
aagggactgg aatgggtggc ctacatctcc tccgactcct
ccgccatcta ctacgccgac accgtgaagg gcaggttcac
catctcccgg gacaacgcca agaactccct gtacctgcag
atgaactccc tgcgggacga ggacaccgcc gtgtactact
gcggcagagg ccgggagaat atctactacg gctcccggct
ggattattgg ggccagggca ccaccgtgac cgtgtcctcc
ggaggatgtg gcggtggaaa agtggccgca ctgaaggaga
aagttgctgc tttgaaagag aaggtcgccg cacttaagga
aaaggtcgca gccctgaaag ag

The third polypeptide chain of DART-A comprises, in the N-terminal to C-terminal direction, an N-terminus, a peptide (Linker 3; DKTHTCPPCP (SEQ ID NO:4)), a “hole-bearing” Fc Domain (SEQ ID NO:16), and a C-terminus.

Thus, the third polypeptide of DART-A is composed of: SEQ ID NO:4-SEQ ID NO:16.

The amino acid sequence of the third polypeptide of DART-A is (SEQ ID NO:57):

DKTHTCPPCP APEAAGGPSV FLFPPKPKDT LMISRTPEVT
CVVVDVSHED PEVKFNWYVD GVEVHNAKTK PREEQYNSTY
RVVSVLTVLH QDWLNGKEYK CKVSNKALPA PIEKTISKAK
GQPREPQVYT LPPSREEMTK NQVSLSCAVK GFYPSDIAVE
WESNGQPENN YKTTPPVLDS DGSFFLVSKL TVDKSRWQQG
NVFSCSVMHE ALHNRYTQKS LSLSPGK

A preferred polynucleotide that encodes such a polypeptide has the sequence (SEQ ID NO:58):

gacaaaactc acacatgccc accgtgccca gcacctgaag
ccgcgggggg accgtcagtc ttcctcttcc ccccaaaacc
caaggacacc ctcatgatct cccggacccc tgaggtcaca
tgcgtggtgg tggacgtgag ccacgaagac cctgaggtca
agttcaactg gtacgtggac ggcgtggagg tgcataatgc
caagacaaag ccgcgggagg agcagtacaa cagcacgtac
cgtgtggtca gcgtcctcac cgtcctgcac caggactggc
tgaatggcaa ggagtacaag tgcaaggtct ccaacaaagc
cctcccagcc cccatcgaga aaaccatctc caaagccaaa
gggcagcccc gagaaccaca ggtgtacacc ctgcccccat
cccgggagga gatgaccaag aaccaggtca gcctgagttg
cgcagtcaaa ggcttctatc ccagcgacat cgccgtggag
tgggagagca atgggcagcc ggagaacaac tacaagacca
cgcctcccgt gctggactcc gacggctcct tcttcctcgt
cagcaagctc accgtggaca agagcaggtg gcagcagggg
aacgtcttct catgctccgt gatgcatgag gctctgcaca
accgctacac gcagaagagc ctctccctgt ctccgggtaa a

B. DART-B

The first polypeptide chain of DART-B comprises, in the N-terminal to C-terminal direction, an N-terminus, a VL domain of a monoclonal antibody capable of binding to B7-H3 (VL_{B7-H3} B7-H3 mAb B) (SEQ ID NO:25), an intervening linker peptide (Linker 1; GGGSGGGG (SEQ ID NO:1)), a VH domain of a monoclonal antibody capable of binding to CD3 (VH_{CD3} CD3 mAb A) (SEQ ID NO:45), an intervening linker peptide (Linker 2; GGCGGG (SEQ ID NO:2)), a Heterodimer-Promoting (E-coil) Domain (EVAALEK-EVAALEK-EVAALEK-EVAALEK (SEQ ID NO:10)), an intervening linker peptide (Spacer-Linker 3; GGGDKTHTCPPCP (SEQ ID NO:5)), a “knob-bearing” Fc Domain (SEQ ID NO:15), and a C-terminus.

Thus, the first polypeptide chain of DART-B is composed of: SEQ ID NO:25-SEQ ID NO:1-SEQ ID NO:45-SEQ ID NO:2-SEQ ID NO:10-SEQ ID NO:5-SEQ ID NO:15.

The amino acid sequence of the first polypeptide of DART-B is (SEQ ID NO:59):

DIQMTQSPSS LSASVGDRVT ITCRASQDIS NYLNWYQQKP
GKAPKLLIYY TSRLHSGVPS RFSGSGSGTD FTLTISSLQP
EDIATYYCQQ GNTLPPTFGG GTKLEIKGGG SGGGGEVQLV
ESGGGLVQPG GSLRLSCAAS GFTFSTYAMN WVRQAPGKGL
EWVGRIRSKY NNYATYYADS VKDRFTISRD DSKNSLYLQM
NSLKTEDTAV YYCVRHGNFG NSYVSWFAYW GQGTLVTVSS
GGCGGGEVAA LEKEVAALEK EVAALEKEVA ALEKGGGDKT
HTCPPCPAPE AAGGPSVFLF PPKPKDTLMI SRTPEVTCVV
VDVSHEDPEV KFNWYVDGVE VHNAKTKPRE EQYNSTYRVV
SVLTVLHQDW LNGKEYKCKV SNKALPAPIE KTISKAKGQP
REPQVYTLPP SREEMTKNQV SLWCLVKGFY PSDIAVEWES
NGQPENNYKT TPPVLDSDGS FFLYSKLTVD KSRWQQGNVF
SCSVMHEALH NHYTQKSLSL SPGK

The second polypeptide chain of DART-B comprises, comprises, in the N-terminal to C-terminal direction, an N-terminus, a VL domain of a monoclonal antibody capable of binding to CD3 (VL_{CD3} CD3 mAb A) (SEQ ID NO:41), an intervening linker peptide (Linker 1; GGGSGGGG (SEQ ID NO:1)), a VH domain of a monoclonal antibody capable of binding to B7-H3 (VH_{B7-H3} B7-H3 mAb B) (SEQ ID NO:29), an intervening linker peptide (Linker 2; GGCGGG (SEQ ID NO:2)), a Heterodimer-Promoting (K-coil) Domain (KVAALKE-KVAALKE-KVAALKE-KVAALKE (SEQ ID NO:11), and a C-terminus.

Thus, the second polypeptide of DART-B is composed of: SEQ ID NO:41-SEQ ID NO:1-SEQ ID NO:29-SEQ ID NO:2-SEQ ID NO:11.

The amino acid sequence of the second polypeptide of DART-B is (SEQ ID NO:60):

QAVVTQEPSL TVSPGGTVTL TCRSSTGAVT TSNYANWVQQ
KPGQAPRGLI GGTNKRAPWT PARFSGSLLG GKAALTITGA
QAEDEADYYC ALWYSNLWVF GGGTKLTVLG GGGSGGGGQV
QLVQSGAEVK KPGASVKVSC KASGYTFTSY WMQWVRQAPG
QGLEWMGTIY PGDGDTRYTQ KFKGRVTITA DKSTSTAYME
LSSLRSEDTA VYYCARRGIP RLWYFDVWGQ GTTVTVSSGG
CGGGKVAALK EKVAALKEKV AALKEKVAAL KE

The third polypeptide chain of DART-B comprises, in the N-terminal to C-terminal direction, an N-terminus, a peptide (Linker 3; DKTHTCPPCP (SEQ ID NO:4)), a “hole-bearing” Fc Domain (SEQ ID NO:16), and a C-terminus.

Thus, the third polypeptide of DART-B is composed of: SEQ ID NO:4-SEQ ID NO:16 and has the same amino acid sequence as the third polypeptide of DART-A (SEQ ID NO:57) provided above.

C. DART-C

The first polypeptide chain of DART-C comprises, in the N-terminal to C-terminal direction, an N-terminus, a VL domain of a monoclonal antibody capable of binding to B7-H3 (VL_{B7-H3} B7-H3 mAb C) (SEQ ID NO:33), an intervening linker peptide (Linker 1; GGGSGGGG (SEQ ID NO:1)), a VH domain of a monoclonal antibody capable of binding to CD3 (VH_{CD3} CD3 mAb A having a D65G substitution) (SEQ ID NO:50), an intervening linker peptide (Linker 2; GGCGGG (SEQ ID NO:2)), a Heterodimer-Promoting (E-coil) Domain (EVAALEK-EVAALEK-EVAALEK-EVAALEK (SEQ ID NO:10)), an intervening linker peptide (Spacer-Linker 3; GGGDKTHTCPPCP (SEQ ID NO:5)), a “knob-bearing” Fc Domain (SEQ ID NO:15), and a C-terminus.

Thus, the first polypeptide chain of DART-C is composed of: SEQ ID NO:33-SEQ ID NO:1-SEQ ID NO:50-SEQ ID NO:2-SEQ ID NO:10-SEQ ID NO:5-SEQ ID NO:15.

The amino acid sequence of the first polypeptide of DART-C is (SEQ ID NO:61):

DIQMTQSPSS LSASVGDRVT ITCRASQSIS SYLNWYQQKP
GKAPKLLIYY TSRLQSGVPS RFSGSGSGTD FTLTISSLQP
EDIATYYCQQ GNTLPPTFGG GTKLEIKGGG SGGGGEVQLV
ESGGGLVQPG GSLRLSCAAS GFTFSTYAMN WVRQAPGKGL
EWVGRIRSKY NNYATYYADS VKGRFTISRD DSKNSLYLQM
NSLKTEDTAV YYCVRHGNFG NSYVSWFAYW GQGTLVTVSS
GGCGGGEVAA LEKEVAALEK EVAALEKEVA ALEKGGGDKT
HTCPPCPAPE AAGGPSVFLF PPKPKDTLMI SRTPEVTCVV
VDVSHEDPEV KFNWYVDGVE VHNAKTKPRE EQYNSTYRVV
SVLTVLHQDW LNGKEYKCKV SNKALPAPIE KTISKAKGQP
REPQVYTLPP SREEMTKNQV SLWCLVKGFY PSDIAVEWES
NGQPENNYKT TPPVLDSDGS FFLYSKLTVD KSRWQQGNVF
SCSVMHEALH NHYTQKSLSL

The second polypeptide chain of DART-C comprises, comprises, in the N-terminal to C-terminal direction, an N-terminus, a VL domain of a monoclonal antibody capable of binding to CD3 (VL_{CD3} CD3 mAb C) (SEQ ID NO:41), an intervening linker peptide (Linker 1; GGGSGGGG (SEQ ID NO:1)), a VH domain of a monoclonal antibody capable of binding to B7-H3 (VH_{B7-H3} B7-H3 mAb B) (SEQ ID NO:37), an intervening linker peptide (Linker 2; GGCGGG (SEQ ID NO:2)), a Heterodimer-Promoting (K-coil) Domain (KVAALKE-KVAALKE-KVAALKE-KVAALKE (SEQ ID NO:11), and a C-terminus.

Thus, the second polypeptide of DART-C is composed of: SEQ ID NO:41-SEQ ID NO:1-SEQ ID NO:37-SEQ ID NO:2-SEQ ID NO:11.

The amino acid sequence of the second polypeptide of DART-C is (SEQ ID NO:62):

QAVVTQEPSL TVSPGGTVTL TCRSSTGAVT TSNYANWVQQ
KPGQAPRGLI GGTNKRAPWT PARFSGSLLG GKAALTITGA
QAEDEADYYC ALWYSNLWVF GGGTKLTVLG GGGSGGGGQV
QLVQSGAEVK KPGASVKVSC KASGYTFTSY WMQWVRQAPG
QGLEWMGTIY PGGGDTRYTQ KFQGRVTITA DKSTSTAYME
LSSLRSEDTA VYYCARRGIP RLWYFDVWGQ GTTVTVSSGG
CGGGKVAALK EKVAALKEKV AALKEKVAAL KE

The third polypeptide chain of DART-C comprises, in the N-terminal to C-terminal direction, an N-terminus, a peptide (Linker 3; DKTHTCPPCP (SEQ ID NO:4)), a “hole-bearing” Fc Domain (SEQ ID NO:16), and a C-terminus.

Thus, the third polypeptide of DART-C is composed of: SEQ ID NO:4-SEQ ID NO:16 and has the same amino acid sequence as the third polypeptide of DART-A (SEQ ID NO:57) provided above.

D. DART-D

The general structure of the first and second polypeptide chains of exemplary DART-D are the same as that of DART-A, DART-B, and DART-C except that DART-D comprises an alternative Linker 2, which lacks a cysteine residue, and comprises cysteine-containing Heterodimer-Promoting Domains. The first polypeptide chain of DART-D comprises, in the N-terminal to C-terminal direction, an N-terminus, a VL domain of a monoclonal antibody capable of binding to B7-H3 (VL_{B7-H3} B7-H3 mAb C) (SEQ ID NO:33), an intervening linker peptide (Linker 1; GGGSGGGG (SEQ ID NO:1)), a VH domain of a monoclonal antibody capable of binding to CD3 (VH_{CD3} CD3 mAb A having a D65G substitution) (SEQ ID NO:50), an intervening linker peptide (Linker 2; ASTKG (SEQ ID NO:3)), a Heterodimer-Promoting (E-coil) Domain (EVAACEK-EVAALEK-EVAALEK-EVAALEK (SEQ ID NO:12)), an intervening linker peptide (Spacer-Linker 3; GGGDKTHTCPPCP (SEQ ID NO:5)), a “knob-bearing” Fc Domain (SEQ ID NO:15), and a C-terminus.

Thus, the first polypeptide chain of DART-D is composed of: SEQ ID NO:33-SEQ ID NO:1-SEQ ID NO:50-SEQ ID NO:3-SEQ ID NO:12-SEQ ID NO:5-SEQ ID NO:15.

The amino acid sequence of the first polypeptide of DART-D is (SEQ ID NO:63):

DIQMTQSPSS LSASVGDRVT ITCRASQSIS SYLNWYQQKP
GKAPKLLIYY TSRLQSGVPS RFSGSGSGTD FTLTISSLQP
EDIATYYCQQ GNTLPPTFGG GTKLEIKGGG SGGGGEVQLV
ESGGGLVQPG GSLRLSCAAS GFTFSTYAMN WVRQAPGKGL
EWVGRIRSKY NNYATYYADS VKGRFTISRD DSKNSLYLQM
NSLKTEDTAV YYCVRHGNFG NSYVSWFAYW GQGTLVTVSS
ASTKGEVAAC EKEVAALEKE VAALEKEVAA LEKGGGDKTH
TCPPCPAPEA AGGPSVFLFP PKPKDTLMIS RTPEVTCVVV
DVSHEDPEVK FNWYVDGVEV HNAKTKPREE QYNSTYRVVS
VLTVLHQDWL NGKEYKCKVS NKALPAPIEK TISKAKGQPR
EPQVYTLPPS REEMTKNQVS LWCLVKGFYP SDIAVEWESN
GQPENNYKTT PPVLDSDGSF FLYSKLTVDK SRWQQGNVFS
CSVMHEALHN HYTQKSLSLS PGK

The second polypeptide chain of DART-D comprises, comprises, in the N-terminal to C-terminal direction, an N-terminus, a VL domain of a monoclonal antibody capable of binding to CD3 (VL_{CD3} CD3 mAb C) (SEQ ID NO:41), an intervening linker peptide (Linker 1; GGGSGGGG (SEQ ID NO:1)), a VH domain of a monoclonal antibody capable of binding to B7-H3 (VH_{B7-H3} B7-H3 mAb B) (SEQ ID NO:37), an intervening linker peptide (Linker 2; ASTKG (SEQ ID NO:3)), a Heterodimer-Promoting (K-coil) Domain (KVAACKE-KVAALKE-KVAALKE-KVAALKE (SEQ ID NO:13), and a C-terminus.

Thus, the second polypeptide of DART-C is composed of: SEQ ID NO:41-SEQ ID NO:1-SEQ ID NO:37-SEQ ID NO:3-SEQ ID NO:13.

The amino acid sequence of the second polypeptide of DART-D is (SEQ ID NO:64):

QAVVTQEPSL TVSPGGTVTL TCRSSTGAVT TSNYANWVQQ
KPGQAPRGLI GGTNKRAPWT PARFSGSLLG GKAALTITGA
QAEDEADYYC ALWYSNLWVF GGGTKLTVLG GGGSGGGGQV
QLVQSGAEVK KPGASVKVSC KASGYTFTSY WMQWVRQAPG
QGLEWMGTIY PGGGDTRYTQ KFQGRVTITA DKSTSTAYME
LSSLRSEDTA VYYCARRGIP RLWYFDVWGQ GTTVTVSSAS
TKGKVAACKE KVAALKEKVA ALKEKVAALK E

While the first and second polypeptide chains incorporate different linkers and Heterodimer-Promoting Domains, the third polypeptide chain of DART-D comprises, in the N-terminal to C-terminal direction, an N-terminus, a peptide (Linker 3; DKTHTCPPCP (SEQ ID NO:4)), a “hole-bearing” Fc Domain (SEQ ID NO:16), and a C-terminus.

Thus, the third polypeptide of DART-D is composed of: SEQ ID NO:4-SEQ ID NO:16 and has the same amino acid sequence as the third polypeptide of DART-A (SEQ ID NO:57) provided above.

### IV. Control DART® Diabodies

In order to more meaningfully demonstrate the properties of the B7-H3×CD3 bispecific monovalent Fc diabodies of the present invention, a control bispecific monovalent Fc diabody was constructed capable of binding to fluorescein and CD3 (designated “Control DART”).

The anti-fluorescein antibody used to form the Control DART® diabody was antibody 4-4-20 (Gruber, M. et al. (1994) “Efficient Tumor Cell Lysis Mediated By A Bispecific Single Chain Antibody Expressed In Escherichia coli,” J. Immunol. 152(11):5368-5374; Bedzyk, W. D. et al. (1989) “Comparison Of Variable Region Primary Structures Within An Anti-Fluorescein Idiotype Family,” J. Biol. Chem. 264(3): 1565-1569) were used in control diabodies. The amino acid sequences of the variable light and variable heavy Domains of anti-fluorescein antibody 4-4-20 are as follows:

The amino acid sequence of the VL Domain of anti-fluorescein antibody 4-4-20 (SEQ ID NO:65) is shown below (CDR_{H} residues are shown underlined):

DVVMTQTPFS LPVSLGDQAS ISCRSSQSLV HSNGNTYLRW
YLQKPGQSPK VLIYKVSNRF SGVPDRFSGS GSGTDFTLKI
SRVEAEDLGV YFCSQSTHVP WTFGGGTKLE IK

The amino acid sequence of the VH Domain of anti-fluorescein antibody 4-4-20 (SEQ ID NO:66) is shown below (CDR_{H} residues are shown underlined):

EVKLDETGGG LVQPGRPMKL SCVASGFTFS DYWMNWVRQS
PEKGLEWVAQ IRNKPYNYET YYSDSVKGRF TISRDDSKSS
VYLQMNNLRV EDMGIYYCTG SYYGMDYWGQ GTSVTVSS

The first polypeptide chain of Control DART comprises, in the N-terminal to C-terminal direction, an N-terminus, a VL domain of a monoclonal antibody capable of binding to fluorescein (VL_{Fluor} 4-4-20) (SEQ ID NO:65), an intervening linker peptide (Linker 1; GGGSGGGG (SEQ ID NO:1)), a VH domain of a monoclonal antibody capable of binding to CD3 (VH_{CD3} CD3 mAb A having a D65G substitution) (SEQ ID NO:50), an intervening linker peptide (Linker 2; GGCGGG (SEQ ID NO:2)), a Heterodimer-Promoting (E-coil) Domain (EVAALEK-EVAALEK-EVAALEK-EVAALEK (SEQ ID NO:10)), an intervening linker peptide (Spacer-Linker 3; GGGDKTHTCPPCP (SEQ ID NO:5)), a “knob-bearing” Fc Domain (SEQ ID NO:15), and a C-terminus.

Thus, the first polypeptide chain of Control DART is composed of: SEQ ID NO:65-SEQ ID NO:1-SEQ ID NO:50-SEQ ID NO:2-SEQ ID NO:10-SEQ ID NO:5-SEQ ID NO:15.

The amino acid sequence of the first polypeptide chain of Control DART is (SEQ ID NO:67):

DVVMTQTPFS LPVSLGDQAS ISCRSSQSLV HSNGNTYLRW
YLQKPGQSPK VLIYKVSNRF SGVPDRFSGS GSGTDFTLKI
SRVEAEDLGV YFCSQSTHVP WTFGGGTKLE IKGGGSGGGG
EVQLVESGGG LVQPGGSLRL SCAASGFTFS TYAMNWVRQA
PGKGLEWVGR IRSKYNNYAT YYADSVKGRF TISRDDSKNS
LYLQMNSLKT EDTAVYYCVR HGNFGNSYVS WFAYWGQGTL
VTVSSGGCGG GEVAALEKEV AALEKEVAAL EKEVAALEKG
GGDKTHTCPP CPAPEAAGGP SVFLFPPKPK DTLMISRTPE
VTCVVVDVSH EDPEVKFNWY VDGVEVHNAK TKPREEQYNS
TYRVVSVLTV LHQDWLNGKE YKCKVSNKAL PAPIEKTISK
AKGQPREPQV YTLPPSREEM TKNQVSLWCL VKGFYPSDIA
VEWESNGQPE NNYKTTPPVL DSDGSFFLYS KLTVDKSRWQ
QGNVFSCSVM HEALHNHYTQ KSLSLSPGK

The second polypeptide chain of Control DART comprises, in the N-terminal to C-terminal direction, an N-terminus, a VL domain of a monoclonal antibody capable of binding to CD3 (VL_{CD3} CD3 mAb A) (SEQ ID NO:37), an intervening linker peptide (Linker 1; GGGSGGGG (SEQ ID NO:1)), a VH domain of a monoclonal antibody capable of binding to or fluorescein (VH_{fluor} 4-4-20) (SEQ ID NO:65), an intervening linker peptide (Linker 2; GGCGGG (SEQ ID NO:2)), a Heterodimer-Promoting (K-coil) Domain (KVAALKE-KVAALKE-KVAALKE-KVAALKE (SEQ ID NO:11), and a C-terminus.

Thus, the second polypeptide chain of Control DART is composed of: SEQ ID NO:37-SEQ ID NO:1-SEQ ID NO:65-SEQ ID NO:2-SEQ ID NO:11.

The amino acid sequence of the second polypeptide chain of Control DART is (SEQ ID NO:68):

QAVVTQEPSL TVSPGGTVTL TCRSSTGAVT TSNYANWVQQ
KPGQAPRGLI GGTNKRAPWT PARFSGSLLG GKAALTITGA
QAEDEADYYC ALWYSNLWVF GGGTKLTVLG GGGSGGGGEV
KLDETGGGLV QPGRPMKLSC VASGFTFSDY WMNWVRQSPE
KGLEWVAQIR NKPYNYETYY SDSVKGRFTI SRDDSKSSVY
LQMNNLRVED MGIYYCTGSY YGMDYWGQGT SVTVSSGGCG
GGKVAALKEK VAALKEKVAA LKEKVAALKE

The third polypeptide chain of Control DART comprises, in the N-terminal to C-terminal direction, an N-terminus, a peptide (Linker 3; DKTHTCPPCP (SEQ ID NO:4)), a “hole-bearing” Fc Domain (SEQ ID NO:16), and a C-terminus.

Thus, the third polypeptide chain of Control DART is composed of: SEQ ID NO:4-SEQ ID NO:16 and has the same amino acid sequence as the third polypeptide of DART-A (SEQ ID NO:57) provided above.

### V. Pharmaceutical Compositions

The compositions of the invention include bulk drug compositions useful in the manufacture of pharmaceutical compositions (e.g., impure or non-sterile compositions) and pharmaceutical compositions (i.e., compositions that are suitable for administration to a subject or patient) which can be used in the preparation of unit dosage forms. Such compositions comprise a B7-H3×CD3 bispecific monovalent Fc diabody of the present invention, or a combination of such agents and a pharmaceutically acceptable carrier. Preferably, compositions of the invention comprise a prophylactically or therapeutically effective amount of the B7-H3×CD3 bispecific monovalent Fc diabody of the invention and a pharmaceutically acceptable carrier.

The invention also encompasses pharmaceutical compositions comprising a B7-H3×CD3 bispecific monovalent Fc diabody of the invention and one or more additional molecules that are effective in stimulating an immune response (e.g., an immune checkpoint inhibitor) and/or in combination with one or more additional molecules that specifically bind a cancer antigen (e.g., tumor specific monoclonal antibody or diabody) that is specific for at least one particular cancer antigen, and a pharmaceutically acceptable carrier. As used herein, the term “cancer antigen” denotes an antigen that is characteristically expressed on the surface of a tumor cell. Examples of cancer antigens include: A33 (a colorectal carcinoma antigen; Almqvist, Y. 2006, Nucl Med Biol. November; 33(8):991-998); B1 (Egloff, A. M. et al. 2006, Cancer Res. 66(1):6-9); BAGE (Bodey, B. 2002 Expert Opin Biol Ther. 2(6):577-84); beta-catenin (Prange W. et al. 2003 J Pathol. 201(2):250-9); CA125 (Bast, R. C. Jr. et al. 2005 Int J Gynecol Cancer 15 Suppl 3:274-81); CD5 (Calin, G. A. et al. 2006 Semin Oncol. 33(2):167-73; CD19 (Troussard, X. et al. 1998 Hematol Cell Ther. 40(4):139-48); CD20 (Thomas, D. A. et al. 2006 Hematol Oncol Clin North Am. 20(5):1125-36); CD22 (Kreitman, R. J. 2006 AAPS J. 18; 8(3):E532-51); CD23 (Rosati, S. et al. 2005 Curr Top Microbiol Immunol. 5; 294:91-107); CD25 (Troussard, X. et al. 1998 Hematol Cell Ther. 40(4):139-48); CD27 (Bataille, R. 2006 Haematologica 91(9):1234-40); CD28 (Bataille, R. 2006 Haematologica 91(9):1234-40); CD36 (Ge, Y. 2005 Lab Hematol. 11(1):31-7); CD40/CD154 (Messmer, D. et al. 2005 Ann N Y Acad Sci. 1062:51-60); CD45 (Jurcic, J. G. 2005 Curr Oncol Rep. 7(5):339-46); CD56 (Bataille, R. 2006 Haematologica 91(9):1234-40); CD79a/CD79b (Troussard, X. et al. 1998 Hematol Cell Ther. 40(4):139-48; Chu, P. G. et al. 2001 Appl Immunohistochem Mol Morphol. 9(2):97-106); CD103 (Troussard, X. et al. 1998 Hematol Cell Ther. 40(4):139-48); CDK4 (Lee, Y. M. et al. 2006 Cell Cycle 5(18):2110-4); CEA (carcinoembryonic antigen; Mathelin, C. 2006 Gynecol Obstet Fertil. 34(7-8):638-46; Tellez-Avila, F. I. et al. 2005 Rev Invest Clin. 57(6):814-9); CTLA4 (Peggs, K. S. et al. 2006 Curr Opin Immunol. 18(2):206-13); EGF-R (epidermal growth factor receptor; Adenis, A. et al. 2003 Bull Cancer. 90 Spec No: S228-32); Erb (ErbB1; ErbB3; ErbB4; Zhou, H. et al. 2002 Oncogene 21(57):8732-40; Rimon, E. et al. 2004 Int J Oncol. 24(5):1325-38); GAGE (GAGE-1; GAGE-2; Akcakanat, A. et al. 2006 Int J Cancer. 118(1):123-8); GD2/GD3/GM2 (Livingston, P. O. et al. 2005 Cancer Immunol Immunother. 54(10):1018-25); gp100 (Lotem, M. et al. 2006 J Immunother. 29(6):616-27); HER-2/neu (Kumar, Pal S et al. 2006 Semin Oncol. 33(4):386-91); human papillomavirus-E6/human papillomavirus-E7 (DiMaio, D. et al. 2006 Adv Virus Res. 66:125-59; KSA (17-1A) (Ragupathi, G. 2005 Cancer Treat Res. 123:157-80); MAGE (MAGE-1; MAGE-3; (Bodey, B. 2002 Expert Opin Biol Ther. 2(6):577-84); MART (Kounalakis, N. et al. 2005 Curr Oncol Rep. 7(5):377-82; MUC-1 (Mathelin, C. 2006 Gynecol Obstet Fertil. 34(7-8):638-46); MUM-1 (Castelli, C. et al. 2000 J Cell Physiol. 182(3):323-31); N-acetylglucosaminyltransferase (Dennis, J. W. 1999 Biochim Biophys Acta. 6; 1473 (1): 21-34); p15 (Gil, J. et al. 2006 Nat Rev Mol Cell Biol. 7(9):667-77); PSA (prostate specific antigen; Cracco, C. M. et al. 2005 Minerva Urol Nefrol. 57(4):301-11); PSMA (Ragupathi, G. 2005 Cancer Treat Res. 123:157-80); sTn (Holmberg, L. A. 2001 Expert Opin Biol Ther. 1(5):881-91); TNF-receptor (TNF-α receptor, TNF-β receptor; or TNF-γ receptor; van Horssen, R. et al. 2006 Oncologist. 11(4):397-408; Gardnerova, M. et al. 2000 Curr Drug Targets. 1(4):327-64); VEGF receptor (O'Dwyer. P. J. 2006 Oncologist. 11(9):992-8); ADAM-9 (United States Patent Publication No. 2006/0172350; PCT Publication No. WO 06/084075); ALCAM (PCT Publication No. WO 03/093443); Carboxypeptidase M (United States Patent Publication No. 2006/0166291); CD46 (U.S. Pat. No. 7,148,038; PCT Publication No. WO 03/032814); Cytokeratin 8 (PCT Publication No. WO 03/024191); Ephrin receptors (and in particular EphA2 (U.S. Pat. No. 7,569,672; PCT Publication No. WO 06/084226); Integrin Alpha-V-Beta-6 (PCT Publication No. WO 03/087340); JAM-3 (PCT Publication No. WO 06/084078); KID3 (PCT Publication No. WO 05/028498); KID31 (PCT Publication No. WO 06/076584); LUCA-2 (United States Patent Publication No. 2006/0172349; PCT Publication No. WO 06/083852); Oncostatin M (Oncostatin Receptor Beta) (U.S. Pat. No. 7,572,896; PCT Publication No. WO 06/084092); PIPA (U.S. Pat. No. 7,405,061; PCT Publication No. WO 04/043239); ROR1 (U.S. Pat. No. 5,843,749); and the Transferrin Receptor (U.S. Pat. No. 7,572,895; PCT Publication No. WO 05/121179).

In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant (e.g., Freund's adjuvant (complete and incomplete), excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Aqueous carriers, such as saline solutions, aqueous dextrose and glycerol solutions are preferred when the pharmaceutical composition is administered intravenously. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain a minor amount of a wetting or emulsifying agent, or a pH buffering agent. These compositions can take the form of solutions, suspensions, emulsion, tablets, pills, capsules, powders, sustained-release formulations and the like.

Generally, the ingredients of compositions of the invention are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compositions of the invention can be formulated as neutral or salt forms. Pharmaceutically acceptable salts include, but are not limited to those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

The invention also provides a pharmaceutical pack or kit comprising one or more containers containing a B7-H3×CD3 bispecific monovalent Fc diabody of the present invention alone or with other agents, preferably with a pharmaceutically acceptable carrier. Additionally, one or more other prophylactic or therapeutic agents useful for the treatment of a disease can also be included in the pharmaceutical pack or kit. The invention also provides a pharmaceutical pack or kit comprising one or more containers filled with one or more of the ingredients of the pharmaceutical compositions of the invention. Optionally associated with such container(s) can be a notice in the form prescribed by a governmental agency regulating the manufacture, use or sale of pharmaceuticals or biological products, which notice reflects approval by the agency of manufacture, use or sale for human administration.

A kit can comprise a B7-H3×CD3 bispecific monovalent Fc diabody of the invention. The kit can further comprise one or more other prophylactic and/or therapeutic agents useful for the treatment of cancer, in one or more containers; and/or the kit can further comprise one or more cytotoxic antibodies that bind one or more cancer antigens. In certain embodiments, the other prophylactic or therapeutic agent is a chemotherapeutic. In other embodiments, the prophylactic or therapeutic agent is a biological or hormonal therapeutic.

### VI. Methods of Administration

The compositions of the present invention may be provided for the treatment, prophylaxis, and amelioration of one or more symptoms associated with cancer or other disease, or disorder by administering to a subject an effective amount of a molecule of the invention, or a pharmaceutical composition comprising a molecule of the invention. In a preferred aspect, such compositions are substantially purified (i.e., substantially free from substances that limit its effect or produce undesired side effects). In a specific embodiment, the subject is an animal, preferably a mammal such as non-primate (e.g., bovine, equine, feline, canine, rodent, etc.) or a primate (e.g., monkey such as, a cynomolgus monkey, human, etc.). In a preferred embodiment, the subject is a human.

Various delivery systems are known and can be used to administer the molecules and compositions of the invention, e.g., encapsulation in liposomes, microparticles, microcapsules, recombinant cells capable of expressing the antibody or fusion protein, receptor-mediated endocytosis (See, e.g., Wu et al. (1987) “Receptor-Mediated In Vitro Gene Transformation By A Soluble DNA Carrier System,” J. Biol. Chem. 262:4429-4432), construction of a nucleic acid as part of a retroviral or other vector, etc.

Methods of administering a molecule of the invention include, but are not limited to, parenteral administration (e.g., intradermal, intramuscular, intraperitoneal, intravenous and subcutaneous), epidural, and mucosal (e.g., intranasal and oral routes). In a specific embodiment, the B7-H3×CD3 bispecific monovalent Fc diabodies of the invention are administered intramuscularly, intravenously, or subcutaneously. The compositions may be administered by any convenient route, for example, by infusion or bolus injection, by absorption through epithelial or mucocutaneous linings (e.g., oral mucosa, rectal and intestinal mucosa, etc.) and may be administered together with other biologically active agents. Administration can be systemic or local. In addition, pulmonary administration can also be employed, e.g., by use of an inhaler or nebulizer, and formulation with an aerosolizing agent. See, e.g., U.S. Pat. Nos. 6,019,968; 5,985,320; 5,985,309; 5,934,272; 5,874,064; 5,855,913; 5,290,540; and 4,880,078; and PCT Publication Nos. WO 92/19244; WO 97/32572; WO 97/44013; WO 98/31346; and WO 99/66903, each of which is incorporated herein by reference in its entirety.

The invention also provides that the B7-H3×CD3 bispecific monovalent Fc diabodies of the invention are packaged in a hermetically sealed container such as an ampoule or sachette indicating the quantity of the molecule. In one embodiment, the B7-H3×CD3 bispecific monovalent Fc diabodies of the invention are supplied as a dry sterilized lyophilized powder or water free concentrate in a hermetically sealed container and can be reconstituted, e.g., with water or saline to the appropriate concentration for administration to a subject. Preferably, the B7-H3×CD3 bispecific monovalent Fc diabodies of the invention are supplied as a dry sterile lyophilized powder in a hermetically sealed container.

The lyophilized B7-H3×CD3 bispecific monovalent Fc diabodies of the present invention should be stored at between 2 and 8° C. in their original container and the molecules should be administered within 12 hours, preferably within 6 hours, within 5 hours, within 3 hours, or within 1 hour after being reconstituted. In an alternative embodiment, B7-H3×CD3 bispecific monovalent Fc diabodies of the invention are supplied in liquid form in a hermetically sealed container indicating the quantity and concentration of the molecule, fusion protein, or conjugated molecule. Preferably, the liquid form of the B7-H3×CD3 bispecific monovalent Fc diabodies of the invention are supplied in a hermetically sealed container.

The amount of the composition of the invention which will be effective in the treatment, prevention or amelioration of one or more symptoms associated with a disorder can be determined by standard clinical techniques. The precise dose to be employed in the formulation will also depend on the route of administration, and the seriousness of the condition, and should be decided according to the judgment of the practitioner and each patient's circumstances. Effective doses may be extrapolated from dose-response curves derived from in vitro or animal model test systems.

As used herein, an “effective amount” of a pharmaceutical composition, in one embodiment, is an amount sufficient to effect beneficial or desired results including, without limitation, clinical results such as decreasing symptoms resulting from the disease attenuating a symptom of disease (e.g., the proliferation of cancer cells, tumor presence, tumor metastases, etc.), thereby increasing the quality of life of those suffering from the disease, decreasing the dose of other medications required to treat the disease, enhancing the effect of another medication such as via targeting and/or internalization, delaying the progression of the disease, and/or prolonging survival of individuals.

Such effective amount can be administered in one or more administrations. For purposes of this invention, an effective amount of drug, compound, or pharmaceutical composition is an amount sufficient to reduce the proliferation of (or the effect of) viral presence and to reduce and/or delay the development of the disease (e.g., cancer) either directly or indirectly. In some embodiments, an effective amount of a drug, compound, or pharmaceutical composition may or may not be achieved in conjunction with another drug, compound, or pharmaceutical composition. Thus, an “effective amount” may be considered in the context of administering one or more chemotherapeutic agents, and a single agent may be considered to be given in an effective amount if, in conjunction with one or more other agents, a desirable result may be or is achieved. While individual needs vary, determination of optimal ranges of effective amounts of each component is within the skill of the art.

For the B7-H3×CD3 bispecific monovalent Fc diabodies encompassed by the invention, the dosage administered to a patient is preferably determined based upon the body weight (kg) of the recipient subject. The dosage administered is typically from at least about 0.01 μg/kg, at least about 0.05 μg/kg, at least about 0.1 μg/kg, at least about 0.2 μg/kg, at least about 0.5 μg/kg, at least about 1 μg/kg, at least about 2 μg/kg, at least about 3 μg/kg, at least about 5 μg/kg, at least about 10 μg/kg, at least about 20 μg/kg, at least about 30 μg/kg, at least about 50 μg/kg, at least about 0.1 mg/kg, at least about 0.15 mg/kg, at least about 0.2 mg/kg, at least about 0.5 mg/kg, at least about 1.0 mg/kg, or more of the subject's body weight.

Treatment of a subject with a therapeutically or prophylactically effective amount of a B7-H3×CD3 bispecific monovalent Fc diabody of the invention can comprise a single treatment or, preferably, a series of treatments that may involve the same or differing dosages. For example, a subject may be treated with a B7-H3×CD3 bispecific monovalent Fc diabody of the invention once a week or once every two weeks for between about 2 to about 120 weeks, or more than 120 weeks. It will be appreciated that the effective dosage of the B7-H3×CD3 bispecific monovalent Fc diabody used for treatment may increase or decrease over the course of a particular treatment.

Preferably the B7-H3×CD3 bispecific monovalent Fc diabody is administered using a course of treatment regimen comprising one or more doses (which may remain unchanged, or may increase or decrease in response to a subject's response to the treatment, wherein the treatment regimen is administered over 2 weeks, 3 weeks, 4 weeks, 6 weeks, 8 or more than 8 weeks. Typically, there are 1, 2, 3, 4, 5 or more than 5 courses of treatment. Each course of treatment may be the same or different from any prior regimen.

In certain embodiments, a dosage regimen comprises a first 6-week cycle in which a B7-H3×CD3 bispecific monovalent Fc diabody is administered to a subject bi-weekly (i.e., once every other week), followed by one or more 8 week cycles in which the B7-H3×CD3 bispecific monovalent Fc diabody is administered to a subject bi-weekly. In certain embodiments, a first 6 week cycle is followed by one, two, three, four, five, six, seven, eight, nine, ten, eleven, twelve, thirteen, fourteen, or more than fourteen 8 week cycles.

In particular embodiments, the dosage of a B7-H3×CD3 bispecific monovalent Fc diabody, administered to a subject is at least about 0.1 μg/kg, 0.3 μg/kg, 1.3 μg/kg, 3 μg/kg, 10 μg/kg, 30 μg/kg, or 100 μg/kg of the subject's body weight. The calculated dose will be administered based on the patient's body weight at baseline. However, a significant (≥10%) change in body weight from baseline or established plateau weight should prompt recalculation of the administered dose.

The dosage and frequency of administration of the B7-H3×CD3 bispecific monovalent Fc diabodies of the present invention may be reduced or altered by enhancing uptake and tissue penetration of the B7-H3×CD3 bispecific monovalent Fc diabodies by modifications such as, for example, lipidation.

The dosage of the B7-H3×CD3 bispecific monovalent Fc diabodies of the invention administered to a patient may be calculated for use as a single agent therapy. Alternatively, the B7-H3×CD3 bispecific monovalent Fc diabodies of the invention are used in combination with other therapeutic compositions such that the dosage administered to a patient is lower than when said molecules are used as a single agent therapy.

The pharmaceutical compositions of the invention may be administered locally to the area in need of treatment; this may be achieved by, for example, and not by way of limitation, local infusion, by injection, or by means of an implant, said implant being of a porous, non-porous, or gelatinous material, including membranes, such as sialastic membranes, or fibers. Preferably, when administering a molecule of the invention, care must be taken to use materials to which the molecule does not absorb.

The compositions of the invention can be delivered in a vesicle, in particular a liposome (See Langer (1990) “New Methods Of Drug Delivery,” Science 249:1527-1533); Treat et al., in LIPOSOMES IN THE THERAPY OF INFECTIOUS DISEASE AND CANCER, Lopez-Berestein and Fidler (eds.), Liss, New York, pp. 353-365 (1989); Lopez-Berestein, ibid., pp. 3 17-327).

The compositions of the invention can be delivered in a controlled-release or sustained-release system. Any technique known to one of skill in the art can be used to produce sustained-release formulations comprising one or more B7-H3×CD3 bispecific monovalent Fc diabodies of the invention. See, e.g., U.S. Pat. No. 4,526,938; PCT publication WO 91/05548; PCT publication WO 96/20698; Ning et al. (1996) “Intratumoral Radioimmunotheraphy Of A Human Colon Cancer Xenograft Using A Sustained-Release Gel,” Radiotherapy & Oncology 39:179-189, Song et al. (1995) “Antibody Mediated Lung Targeting Of Long-Circulating Emulsions,” PDA Journal of Pharmaceutical Science & Technology 50:372-397; Cleek et al. (1997) “Biodegradable Polymeric Carriers For A bFGF Antibody For Cardiovascular Application,” Pro. Int'l. Symp. Control. Rel. Bioact. Mater. 24:853-854; and Lam et al. (1997) “Microencapsulation Of Recombinant Humanized Monoclonal Antibody For Local Delivery,” Proc. Int'l. Symp. Control Rel. Bioact. Mater. 24:759-760, each of which is incorporated herein by reference in its entirety. In one embodiment, a pump may be used in a controlled-release system (See Langer, supra; Sefton, (1987) “Implantable Pumps,” CRC Crit. Rev. Biomed. Eng. 14:201-240; Buchwald et al. (1980) “Long-Term, Continuous Intravenous Heparin Administration By An Implantable Infusion Pump In Ambulatory Patients With Recurrent Venous Thrombosis,” Surgery 88:507-516; and Saudek et al. (1989) “A Preliminary Trial Of The Programmable Implantable Medication System For Insulin Delivery,” N. Engl. J. Med. 321:574-579). In another embodiment, polymeric materials can be used to achieve controlled-release of the molecules (see e.g., MEDICAL APPLICATIONS OF CONTROLLED RELEASE, Langer and Wise (eds.), CRC Pres., Boca Raton, Fla. (1974); CONTROLLED DRUG BIOAVAILABILITY, DRUG PRODUCT DESIGN AND PERFORMANCE, Smolen and Ball (eds.), Wiley, New York (1984); Levy et al. (1985) “Inhibition Of Calcification Of Bioprosthetic Heart Valves By Local Controlled-Release Diphosphonate,” Science 228:190-192; During et al. (1989) “Controlled Release Of Dopamine From A Polymeric Brain Implant: In Vivo Characterization,” Ann. Neurol. 25:351-356; Howard et al. (1989) “Intracerebral Drug Delivery In Rats With Lesion-Induced Memory Deficits,” J. Neurosurg. 7(1):105-112); U.S. Pat. No. 5,679,377; U.S. Pat. No. 5,916,597; U.S. Pat. No. 5,912,015; U.S. Pat. No. 5,989,463; U.S. Pat. No. 5,128,326; PCT Publication No. WO 99/15154; and PCT Publication No. WO 99/20253). Examples of polymers used in sustained-release formulations include, but are not limited to, poly(-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. A controlled-release system can be placed in proximity of the therapeutic target (e.g., the lungs), thus requiring only a fraction of the systemic dose (see, e.g., Goodson, in MEDICAL APPLICATIONS OF CONTROLLED RELEASE, supra, vol. 2, pp. 115-138 (1984)). Polymeric compositions useful as controlled-release implants can be used according to Dunn et al. (See U.S. Pat. No. 5,945,155). This particular method is based upon the therapeutic effect of the in situ controlled-release of the bioactive material from the polymer system. The implantation can generally occur anywhere within the body of the patient in need of therapeutic treatment. A non-polymeric sustained delivery system can be used, whereby a non-polymeric implant in the body of the subject is used as a drug delivery system. Upon implantation in the body, the organic solvent of the implant will dissipate, disperse, or leach from the composition into surrounding tissue fluid, and the non-polymeric material will gradually coagulate or precipitate to form a solid, microporous matrix (See U.S. Pat. No. 5,888,533).

Controlled-release systems are discussed in the review by Langer (1990, “New Methods Of Drug Delivery,” Science 249:1527-1533). Any technique known to one of skill in the art can be used to produce sustained-release formulations comprising one or more therapeutic agents of the invention. See, e.g., U.S. Pat. No. 4,526,938; International Publication Nos. WO 91/05548 and WO 96/20698; Ning et al. (1996) “Intratumoral Radioimmunotheraphy Of A Human Colon Cancer Xenograft Using A Sustained-Release Gel,” Radiotherapy & Oncology 39:179-189, Song et al. (1995) “Antibody Mediated Lung Targeting Of Long-Circulating Emulsions,” PDA Journal of Pharmaceutical Science & Technology 50:372-397; Cleek et al. (1997) “Biodegradable Polymeric Carriers For A bFGF Antibody For Cardiovascular Application,” Pro. Int'l. Symp. Control. Rel. Bioact. Mater. 24:853-854; and Lam et al. (1997) “Microencapsulation Of Recombinant Humanized Monoclonal Antibody For Local Delivery,” Proc. Int'l. Symp. Control Rel. Bioact. Mater. 24:759-760, each of which documents is incorporated herein by reference in its entirety.

Where the composition of the invention is a nucleic acid encoding a B7-H3×CD3 bispecific monovalent Fc diabody of the invention, the nucleic acid can be administered in vivo to promote expression of its encoded B7-H3×CD3 bispecific monovalent Fc diabody, by constructing it as part of an appropriate nucleic acid expression vector and administering it so that it becomes intracellular, e.g., by use of a retroviral vector (See U.S. Pat. No. 4,980,286), or by direct injection, or by use of microparticle bombardment (e.g., a gene gun; Biolistic, Dupont), or coating with lipids or cell-surface receptors or transfecting agents, or by administering it in linkage to a homeobox-like peptide which is known to enter the nucleus (See e.g., Joliot et al. (1991) “Antennapedia Homeobox Peptide Regulates Neural Morphogenesis,” Proc. Natl. Acad. Sci. (U.S.A.) 88:1864-1868), etc. Alternatively, a nucleic acid can be introduced intracellularly and incorporated within host cell DNA for expression by homologous recombination.

Treatment of a subject with a therapeutically or prophylactically effective amount of a B7-H3×CD3 bispecific monovalent Fc diabody of the invention can include a single treatment or, preferably, can include a series of treatments. In a preferred example, a subject is treated with such a diabody one time per week, one time bi-weekly (i.e., once every other week), or one time every three weeks, for between about 1 to 52 weeks. The pharmaceutical compositions of the invention can be administered once a day, twice a day, or three times a day. Alternatively, the pharmaceutical compositions can be administered once a week, twice a week, once every two weeks, once a month, once every six weeks, once every two months, twice a year or once per year. It will also be appreciated that the effective dosage of the molecules used for treatment may increase or decrease over the course of a particular treatment.

### VII. Uses of the Compositions of the Invention

The B7-H3×CD3 bispecific monovalent Fc diabodies of the present invention have the ability to co-localize T cells to B7-H3-expressing cells, and thus may be used to treat any disease or condition associated with or characterized by the expression of B7-H3. Thus, without limitation, pharmaceutical compositions comprising such molecules may be employed in the diagnosis or treatment of cancers including cancers characterized by the presence of a cancer cell, including but not limited to a cell of an acute myeloid leukemia, an adrenal gland tumor, an AIDS-associated cancer, an alveolar soft part sarcoma, an astrocytic tumor, bladder cancer, bone cancer, a brain and spinal cord cancer, a metastatic brain tumor, a breast cancer, a carotid body tumors, a cervical cancer, a chondrosarcoma, a chordoma, a chromophobe renal cell carcinoma, a clear cell carcinoma, a colon cancer, a colorectal cancer, a cutaneous benign fibrous histiocytoma, a desmoplastic small round cell tumor, an ependymoma, a Ewing's tumor, an extraskeletal myxoid chondrosarcoma, a fibrogenesis imperfecta ossium, a fibrous dysplasia of the bone, a gallbladder or bile duct cancer, gastric cancer, a gestational trophoblastic disease, a germ cell tumor, a head and neck cancer, hepatocellular carcinoma, a glioblastoma, an islet cell tumor, a Kaposi's Sarcoma, a kidney cancer, a leukemia, a lipoma/benign lipomatous tumor, a liposarcoma/malignant lipomatous tumor, a liver cancer, a lymphoma, a lung cancer, a medulloblastoma, a melanoma, a meningioma, a malignant mesothelioma, a multiple endocrine neoplasia, a multiple myeloma, a myelodysplastic syndrome, a neuroblastoma, a neuroendocrine tumors, a non-small cell lung cancer, an ovarian cancer, a pancreatic cancer, a pharyngeal cancer, a papillary thyroid carcinoma, a parathyroid tumor, a pediatric cancer, a peripheral nerve sheath tumor, a phaeochromocytoma, a pituitary tumor, a prostate cancer, a posterious uveal melanoma, a rare hematologic disorder, a renal cell carcinoma, a renal metastatic cancer, a rhabdoid tumor, a rhabdomysarcoma, a sarcoma, a skin cancer, a soft-tissue sarcoma, a squamous cell cancer, a stomach cancer, a synovial sarcoma, a testicular cancer, a thymic carcinoma, a thymoma, a thyroid metastatic cancer, or a uterine cancer.

In particular, B7-H3×CD3 bispecific monovalent Fc diabodies of the present invention are useful for the treatment of squamous cell cancers of the head and neck (SCCHN), bladder cancers, breast cancers, colorectal cancers, gastric cancers, glioblastomas, kidney cancers, lung cancers including non-small cell lung cancers (NSCLC), melanomas, ovarian cancers, pancreatic cancers, pharyngeal cancers, prostate cancers, renal cell carcinomas, and small round blue cell tumors of childhood including neuroblastomas and rhabdomyosarcomas, each of which highly express B7-H3.

The B7-H3×CD3 bispecific monovalent Fc diabodies of the present invention may additionally be used in the manufacture of medicaments for the treatment of the above-described conditions.

Having now generally described the invention, the same will be more readily understood through reference to the following Embodiments, which are provided by way of illustration and are not intended to be limiting of the present invention unless specified.

### EMBODIMENTS OF THE INVENTION

Provided hereafter are non-limiting examples of certain embodiments of the invention.

### Embodiment 1

A B7-H3×CD3 bispecific monovalent Fc diabody, wherein the bispecific monovalent Fc diabody is capable of specific binding to an epitope of B7-H3 and to an epitope of CD3, and possesses an IgG Fc Domain, wherein the bispecific monovalent Fc diabody comprises a first polypeptide chain, a second polypeptide chain and a third polypeptide chain, wherein the first and second polypeptide chains are covalently bonded to one another and the first and third polypeptide chains are covalently bonded to one another, and wherein:
I. the first polypeptide chain comprises, in the N-terminal to C-terminal direction:
   A. a Domain IA, comprising:
      (1) a sub-Domain (IA1), which comprises a VL Domain capable of binding to either B7-H3 (VL_{B7-H3}) or CD3 (VL_{CD3}); and
      (2) a sub-Domain (IA2), which comprises a VH Domain capable of binding to either B7-H3 (VH_{B7-H3}) or CD3 (VH_{CD3});
      wherein the sub-Domains IA1 and IA2 are separated from one another by a polypeptide linker of 12 or less amino acid residues, and are coordinately selected, such that:
      (a) the sub-Domain IA1 comprises the VL Domain capable of binding to B7-H3 (VL_{B7-H3}) and the sub-Domain IA2 is selected to comprise the VH Domain capable of binding to CD3 (VH_{CD3}); or
      (b) the sub-Domain IA1 comprises the VL Domain capable of binding to CD3 (VL_{CD3}) and the sub-Domain IA2 is selected to comprise the VH Domain capable of binding to B7-H3 (VH_{B7-H3}); and
   B. an optionally present Domain IB, comprising a polypeptide linker linked to a Heterodimer-Promoting Domain;
   C. a Domain IC, comprising a polypeptide linker linked to a CH2-CH3 Domain of an antibody;
II. the second polypeptide chain comprises, in the N-terminal to C-terminal direction:
   A. a Domain IIA, comprising:
      (1) a sub-Domain (IIA1), which comprises a VL Domain capable of binding to either B7-H3 (VL_{B7-H3}) or CD3 (VL_{CD3}); and
      (2) a sub-Domain (IIA2), which comprises a VH Domain capable of binding to either B7-H3 (VH_{B7-H3}) or CD3 (VH_{CD3});
      wherein the sub-Domains IIA1 and IIA2 are separated from one another by a polypeptide linker of 12 or less amino acid residues, and are coordinately selected, such that:
      (a) when the sub-Domain IA1 comprises the VL Domain capable of binding to B7-H3 (VL_{B7-H3}), the sub-Domain IIA1 is selected to comprise the VL Domain capable of binding to CD3 (VL_{CD3}) and the sub-Domain IIA2 is selected to comprise the VH Domain capable of binding to B7-H3 (VH_{B7-H3}); and
      (b) when the sub-Domain IA1 comprises the VL Domain capable of binding to CD3 (VL_{CD3}), then sub-Domain IIA1 is selected to comprise the VL Domain capable of binding to B7-H3 (VL_{B7-H3}) and the sub-Domain IIA2 is selected to comprise the VH Domain capable of binding to CD3 (VH_{CD3});
   B. an optionally present Domain IIB, comprising a polypeptide linker linked to a Heterodimer-Promoting Domain;
III. the third polypeptide chain comprises, in the N-terminal to C-terminal direction a Domain IIIC that comprises a polypeptide linker linked to a CH2-CH3 Domain of an antibody;
   wherein:
(A) (1) at least one of the optionally present Domain IB and the optionally present Domain JIB is present, and wherein the Domain IB or JIB that is present has a positive or negative charge; or
   (2) both the optionally present Domain D3 and the optionally present Domain IIB are present, wherein:
      (i) one of the Domain 1B and the Domain IIB has a Heterodimer-Promoting Domain having a positive charge, and the other of the Domain 1B and the Domain JIB has a Heterodimer-Promoting Domain having a negative charge; or
      (ii) one of the Domain 1B and the Domain IIB has a Heterodimer-Promoting Domain that comprises the amino acid sequence GVEPKSC (SEQ ID NO:6) or VEPKSC (SEQ ID NO:7), and the other of the Domain 1B and the Domain IIB has a Heterodimer-Promoting Domain that comprises the amino acid sequence GFNRGEC (SEQ ID NO:8) or FNRGEC (SEQ ID NO:9);
   (B) the VL_{B7-H3} and the VH_{B7-H3} interact to form an epitope-binding domain capable of binding an epitope of B7-H3, and the VL_{CD3} and VH_{CD3} form an epitope-binding domain capable of binding an epitope of CD3; and
   (C) the CH2-CH3 Domains of the first and third polypeptide chains form an Fc Domain capable of binding to an Fc receptor.

### Embodiment 2

The B7-H3×CD3 bispecific monovalent Fc diabody of embodiment 1, which is capable of cross-reacting with both human and primate B7-H3 and CD3.

### Embodiment 3

The B7-H3×CD3 bispecific monovalent Fc diabody of embodiment 1 or 2, wherein:
(A) (1) the Domains IB and IIB each comprise a cysteine residue that covalently bond the first polypeptide chain to the second polypeptide chain via a disulfide bond; and
   (2) the Domains IC and IIIC each comprise a cysteine residue that covalently bonds the first polypeptide chain to the third polypeptide chain via a disulfide bond; or
(B) (1) the polypeptide linkers separating the Domains IA2 and IB and the Domains IIA2 and IIB each comprise a cysteine residue that covalently bonds the first polypeptide chain to the second polypeptide chain via a disulfide bond; and
   (2) the Domains IC and IIIC each comprise a cysteine residue that covalently bonds the first polypeptide chain to the third polypeptide chain via a disulfide bond.

### Embodiment 4

The B7-H3×CD3 bispecific monovalent Fc diabody of any one of embodiments 1-3, wherein:
(A) the VL_{B7-H3} has the amino acid sequence of SEQ ID NO:17 and the VH_{B7-H3} has the amino acid sequence of SEQ ID NO:21; or
(B) the VL_{B7-H3} has the amino acid sequence of SEQ ID NO:25 and the VH_{B7-H3} has the amino acid sequence of SEQ ID NO:29; or
(C) the VL_{B7-H3} has the amino acid sequence of SEQ ID NO:33 and the VH_{B7-H3} has the amino acid sequence of SEQ ID NO:37.

### Embodiment 5

The B7-H3×CD3 bispecific monovalent Fc diabody of any one of embodiments 1-4, wherein the VL_{CD3} has the amino acid sequence of SEQ ID NO:41 and the VH_{CD3} has the amino acid sequence of SEQ ID NO:45 or 50.

### Embodiment 6

The B7-H3×CD3 bispecific monovalent Fc diabody of any one of embodiments 1-5, wherein:
(A) the CH2-CH3 Domain of the Domain IC has the amino acid sequence of SEQ ID NO:15 and the CH2-CH3 Domain of the Domain IIIC has the amino acid sequence of SEQ ID NO:16; or
(B) the CH2-CH3 Domain of the Domain IC has the amino acid sequence of SEQ ID NO:16 and the CH2-CH3 Domain of the Domain IIIC has the amino acid sequence of SEQ ID NO:15.

### Embodiment 7

The B7-H3×CD3 bispecific monovalent Fc diabody of any one of embodiments 1-6, wherein:
(A) the polypeptide linker separating the sub-Domains IA1 and IA2 has the amino acid sequence of SEQ ID NO:1; and/or
(B) the polypeptide linker separating the sub-Domains IIA1 and IIA2 has the amino acid sequence of SEQ ID NO:1.

### Embodiment 8

The B7-H3×CD3 bispecific monovalent Fc diabody of any one of embodiments 1-7, wherein:
(A) the Heterodimer-Promoting Domain of the Domain IB has the amino acid sequence of SEQ ID NO:10 and the Heterodimer-Promoting Domain of the Domain IIB has the amino acid sequence of SEQ ID NO:11; or
(B) the Heterodimer-Promoting Domain of the Domain IB has the amino acid sequence of SEQ ID NO:11 and the Heterodimer-Promoting Domain of the Domain IIB has the amino acid sequence of SEQ ID NO:10; or
(C) the Heterodimer-Promoting Domain of the Domain IB has the amino acid sequence of SEQ ID NO:12 and the Heterodimer-Promoting Domain of the Domain IIB has the amino acid sequence of SEQ ID NO:13; or
(D) the Heterodimer-Promoting Domain of the Domain IB has the amino acid sequence of SEQ ID NO:13 and the Heterodimer-Promoting Domain of the Domain IIB has the amino acid sequence of SEQ ID NO:12.

### Embodiment 9

The B7-H3×CD3 bispecific monovalent Fc diabody of any one of embodiments 1-8, wherein:
(A) the polypeptide linker separating the Domains IB and IA has the amino acid sequence of SEQ ID NO:2 or 3; and/or
(B) the polypeptide linker separating the sub-Domains IIB and IIA has the amino acid sequence of SEQ ID NO:2 or SEQ ID NO:3.

### Embodiment 10

The B7-H3×CD3 bispecific monovalent Fc diabody of any one of embodiments 1-9, wherein the Domain IC further comprises a polypeptide linker that separates the CH2-CH3 Domain from the Domain IB, and wherein the Domain IIIC further comprises a polypeptide linker N-terminal to the CH2-CH3 Domain.

### Embodiment 11

The B7-H3×CD3 bispecific monovalent Fc diabody of embodiment 10, wherein the polypeptide linkers of Domains IC and IIC have the amino acid sequence of SEQ ID NO:4 or 5.

### Embodiment 12

The B7-H3×CD3 bispecific monovalent Fc diabody of any one of embodiments 1-11, wherein:
(A) the first polypeptide chain has the amino acid sequence of SEQ ID NO:53, the second polypeptide chain has the amino acid sequence of SEQ ID NO:55, and the third polypeptide chain has the amino acid sequence of SEQ ID NO:57; or
(B) the first polypeptide chain has the amino acid sequence of SEQ ID NO:59, the second polypeptide chain has the amino acid sequence of SEQ ID NO:60 and the third polypeptide chain has the amino acid sequence of SEQ ID NO:57; or
(C) the first polypeptide chain has the amino acid sequence of SEQ ID NO:61, the second polypeptide chain has the amino acid sequence of SEQ ID NO:62, and the third polypeptide chain has the amino acid sequence of SEQ ID NO:57; or
(D) the first polypeptide chain has the amino acid sequence of SEQ ID NO:63, the second polypeptide chain has the amino acid sequence of SEQ ID NO:64, and the third polypeptide chain has the amino acid sequence of SEQ ID NO:57.

### Embodiment 13

The B7-H3×CD3 bi-specific monovalent Fc diabody of any one of embodiments 1-12, which is capable of mediating redirected killing of target tumor cells using human T cells in an assay employing a target human tumor cell line selected from the group consisting of: A498 (kidney cancer), JIMT-1/Luc (breast cancer), A375 (melanoma); 22Rv1 (prostate cancer), Detroit562 (pharyngeal cancer), DU145 (prostate cancer); BxPC3 (pancreatic cancer), SKMES-1 (lung cancer), and U87 (glioblastoma), and using purified human primary T cells as effector cells at an Effector cell to T cell ratio of 1:1, 5:1, or 10:1, wherein the observed EC50 of such redirected killing is about 1.5 μg/mL or less, about 1.0 μg/mL or less, about 500 ng/mL or less, about 300 ng/mL or less, about 200 ng/mL or less, about 100 ng/mL or less, about 50 ng/mL or less.

### Embodiment 14

The B7-H3×CD3 bi-specific monovalent Fc diabody of embodiment 13 wherein, target tumor cell killing is measured using a lactate dehydrogenase (LDH) release assay in which the enzymatic activity of LDH released from cells upon cell death is quantitatively measured, or by a luciferase assay in which luciferase relative light unit (RLU) is the read-out to indicate relative viability of Raji/GF target cells, which have been engineered to express both the green fluorescent protein (GFP) and luciferase reporter genes.

### Embodiment 15

The B7-H3×CD3 bi-specific monovalent Fc diabody of any one of embodiments 1-14, which is capable of mediating the inhibition of human tumor growth in a co-mix xenograft in which such molecules are introduced into NOD/SCID mice along with 22Rv1 (human prostate cancer) or A498 (human kidney cancer) tumor cells and activated human T cells at a ratio of 5:1.

### Embodiment 16

The B7-H3×CD3 bi-specific monovalent Fc diabody of any one of embodiments 1-15, which is capable of mediating the inhibition of human tumor growth and/or exhibiting anti-tumor activity in a in an xenograft model in female NSG B2m−/− mice:
(A) implanted with human PBMCs (1×10⁷) by intraperitoneal (IP) injection on Day −1 and Detroit (human pharyngeal cancer tumor cells (5×10⁶) intradermally (ID) on Day 0, and administration of diabody on or Days 20, 22, 23, 26, 28, 30, 33, 35, and 37; or
(B) implanted with A498 (human kidney cancer tumor cells (5×10⁶) intradermally (ID) on Day 0, and human PBMCs (1×10⁷) by intraperitoneal (IP) injection on Day 13 and administration of diabody on Days 33, 35, 36, 39, 41, 43, 46, 48, and 50.

### Embodiment 17

The B7-H3×CD3 bi-specific monovalent Fc diabody of embodiment 15 or 16, wherein the B7-H3×CD3 bi-specific monovalent Fc diabody inhibits tumor growth and/or exhibits anti-tumor activity when provided at a concentration of greater than about 0.5 mg/kg, at a concentration of about 0.5 mg/kg, at a concentration about 0.2 mg/kg, at a concentration of about 0.1 mg/kg, at a concentration of about 0.05 mg/kg, at a concentration of about 0.02 mg/kg, at a concentration of about 0.01 mg/kg, or at a concentration of about 0.005 mg/kg, or at a concentration less than 0.005 mg/kg.

### Embodiment 18

The B7-H3×CD3 bispecific monovalent Fc diabody of any one of embodiments 1-17, for use as a pharmaceutical.

### Embodiment 19

A pharmaceutical composition comprising the B7-H3×CD3 bispecific monovalent Fc diabody of any one of embodiments 1-17 and a physiologically acceptable carrier.

### Embodiment 20

Use of the pharmaceutical composition of embodiment 19 in the treatment of a disease or condition associated with or characterized by the expression of B7-H3.

### Embodiment 21

The use of embodiment 20, wherein the disease or condition associated with or characterized by the expression of B7-H3 is cancer.

### Embodiment 22

The use of embodiment 21, wherein the cancer is characterized by the presence of a cancer cell selected from the group consisting of a cell of: an acute myeloid leukemia, an adrenal gland tumor, an AIDS-associated cancer, an alveolar soft part sarcoma, an astrocytic tumor, bladder cancer, bone cancer, a brain and spinal cord cancer, a metastatic brain tumor, a breast cancer, a carotid body tumors, a cervical cancer, a chondrosarcoma, a chordoma, a chromophobe renal cell carcinoma, a clear cell carcinoma, a colon cancer, a colorectal cancer, a cutaneous benign fibrous histiocytoma, a desmoplastic small round cell tumor, an ependymoma, a Ewing's tumor, an extraskeletal myxoid chondrosarcoma, a fibrogenesis imperfecta ossium, a fibrous dysplasia of the bone, a gallbladder or bile duct cancer, gastric cancer, a gestational trophoblastic disease, a germ cell tumor, a head and neck cancer, hepatocellular carcinoma, a glioblastoma, an islet cell tumor, a Kaposi's Sarcoma, a kidney cancer, a leukemia, a lipoma/benign lipomatous tumor, a liposarcoma/malignant lipomatous tumor, a liver cancer, a lymphoma, a lung cancer, a medulloblastoma, a melanoma, a meningioma, a malignant mesothelioma, a multiple endocrine neoplasia, a multiple myeloma, a myelodysplastic syndrome, a neuroblastoma, a neuroendocrine tumors, a non-small cell lung cancer, an ovarian cancer, a pancreatic cancer, a pharyngeal cancer, a papillary thyroid carcinoma, a parathyroid tumor, a pediatric cancer, a peripheral nerve sheath tumor, a phaeochromocytoma, a pituitary tumor, a prostate cancer, a posterious uveal melanoma, a rare hematologic disorder, a renal cell carcinoma, a renal metastatic cancer, a rhabdoid tumor, a rhabdomysarcoma, a sarcoma, a skin cancer, a soft-tissue sarcoma, a squamous cell cancer, a stomach cancer, a synovial sarcoma, a testicular cancer, a thymic carcinoma, a thymoma, a thyroid metastatic cancer, and a uterine cancer.

### Embodiment 23

The use of embodiment 22, wherein the cancer is selected from the group consisting: bladder cancer, breast cancer, colorectal cancer, gastric cancer, glioblastoma, kidney cancer, lung cancer, melanoma, neuroblastoma, ovarian cancer, pancreatic cancer, pharyngeal cancer, prostate cancer, renal cell carcinoma, rhabdomyosarcoma, and squamous cell cancer of the head and neck (SCCHN).

### Embodiment 24

The use of any one of embodiments 21-23, wherein the B7-H3×CD3 bispecific monovalent Fc diabody is administered to a subject bi-weekly (i.e., every other week).

### Embodiment 25

The use of embodiment 24, wherein the B7-H3×CD3 bispecific monovalent Fc diabody is administered at a dosage of 0.1 μg/kg, 0.3 μg/kg, 1.3 μg/kg, 3 μg/kg, 10 μg/kg, 30 μg/kg, or 100 μg/kg of the subject's body weight at baseline.

Having now generally described the invention, the same will be more readily understood through reference to the following examples, which are provided by way of illustration and are not intended to be limiting of the present invention unless specified.

### EXAMPLES

The following examples illustrate various methods for compositions in the diagnostic or treatment methods of the invention. The examples are intended to illustrate, but in no way limit, the scope of the invention.

### Example 1

### Binding Affinity

The binding affinity of several exemplary B7-H3×CD3 bispecific monovalent Fc diabodies (DART-A, DART-B, and DART-C) to human or cynomolgus monkey B7-H3, was evaluated by BIACORE™ analyses. BIACORE™ analyses measure the dissociation off-rate, kd. The binding affinity (KD) between an antibody and its target is a function of the kinetic constants for association (on rate, ka) and dissociation (off-rate, kd) according to the formula: KD=[kd]/[ka]. The BIACORE™ analysis uses surface plasmon resonance to directly measure these kinetic parameters.

The results of binding of DART-A, DART-B, and DART-C to human and cynomolgus monkey B7-H3, analyzed by surface plasmon resonance (SPR) technology (BIAcore), are shown in Table 2.

TABLE 2

| | | ka (±SD) | kd (±SD) | KD (±SD) |
|---|---|---|---|---|
| DART | Antigens | (M−1s−1) | (s−1) | (nM) |
| DART-A | Human B7-H3 | 2.3 × 10⁵ | 5.6 × 10⁻³ | 24.6 |
| DART-A | Cynomolgus B7-H3 | 1.7 × 10⁵ | 5.1 × 10⁻³ | 30.2 |
| DART-B | Human B7-H3 | 2.4 × 10⁶ | 7.6 × 10⁻³ | 3.2 |
| DART-B | Cynomolgus B7-H3 | 21.5 × 10⁶ | 13.3 × 10⁻³ | 8.9 |
| DART-C | Human B7-H3 | 4.6 × 10⁶ | 7.2 × 10⁻³ | 1.6 |
| DART-C | Cynomolgus B7-H3 | n.d. | n.d. | n.d. |

n.d.—BIAcore analysis not done

The binding affinity of DART-A is similar for human and cynomolgus monkey B7-H3 (KD=24.6 nM and 30.2 nM, respectively). The binding affinity of DART-B for human B7-H3 is within three fold of the affinity for cynomolgus monkey B7-H3 is (KD=3.2 nM and 8.9 nM, respectively). However, DART-B has an approximate 8 fold higher affinity for human B7-H3 than DART-A (KD=3.2 nM and 24.6 nM, respectively). Similarly, DART-C has an approximate 15 fold higher affinity for human B7-H3 than DART-A. DART-D comprises the same B7-H3 binding domains as DART-C, and is expected to have the same binding affinity for B7-H3. The B7-H3 binding domains of DART-C and DART-D were shown to bind to cynomolgus monkey B7-H3 in other studies. The binding affinities (KD) for human and cynomolgus monkey CD3 are nearly identical (˜14 nM). In view of the similar binding affinities for human and cynomolgus monkey B7-H3 the cynomolgus monkey is a relevant species for toxicology evaluations.

### Example 2

### FACS Analysis of B7-H3 Expression on Tumor Cell Lines

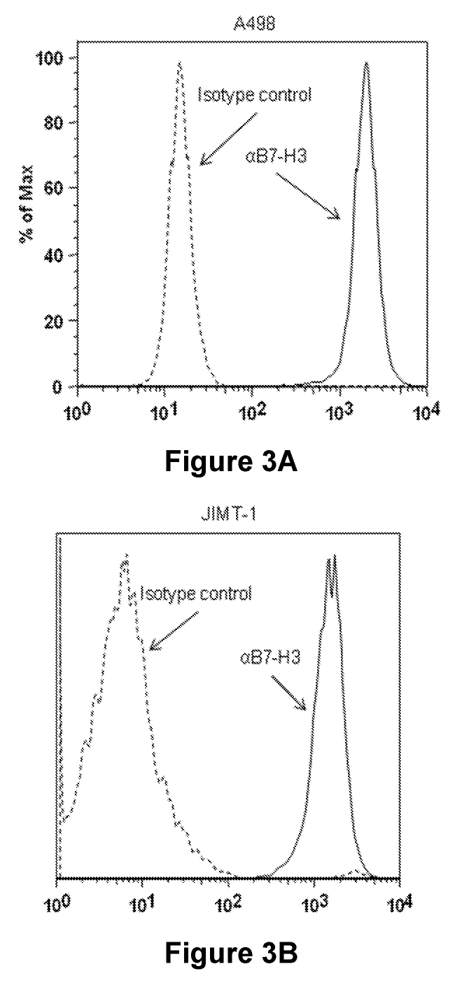

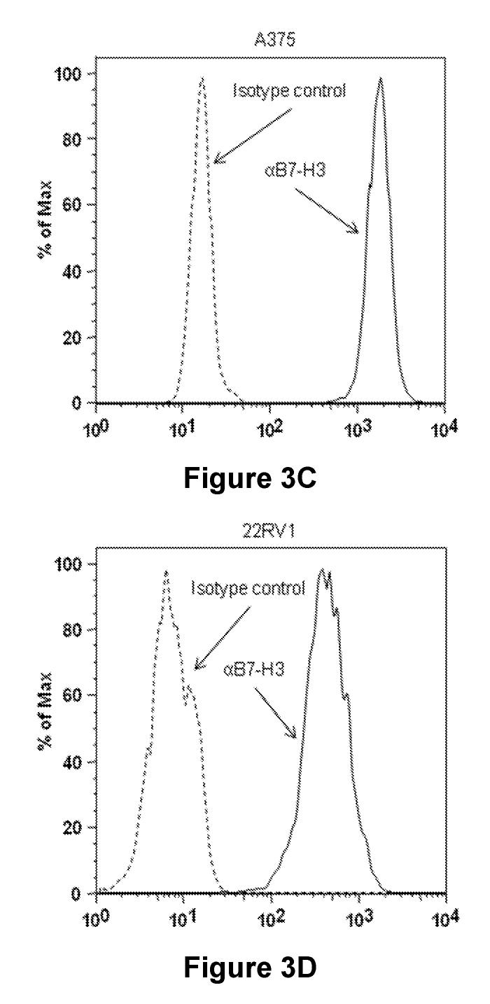

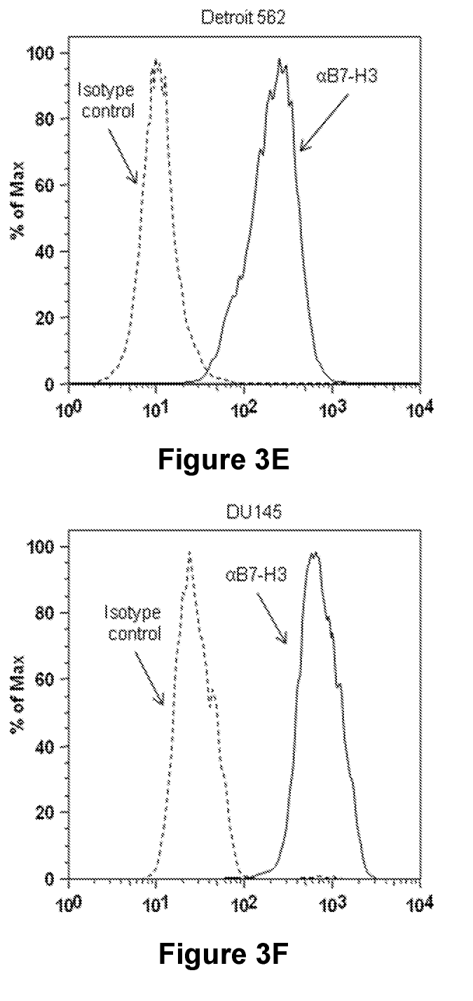

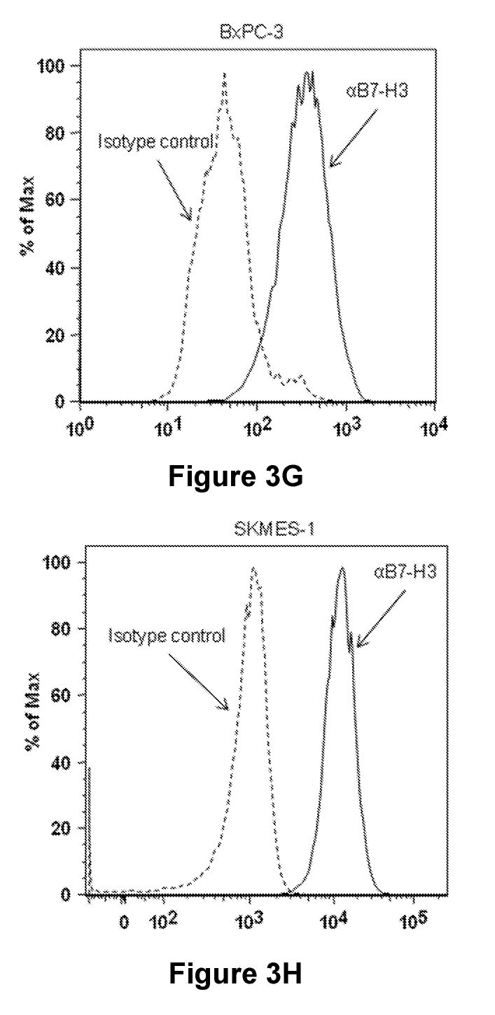

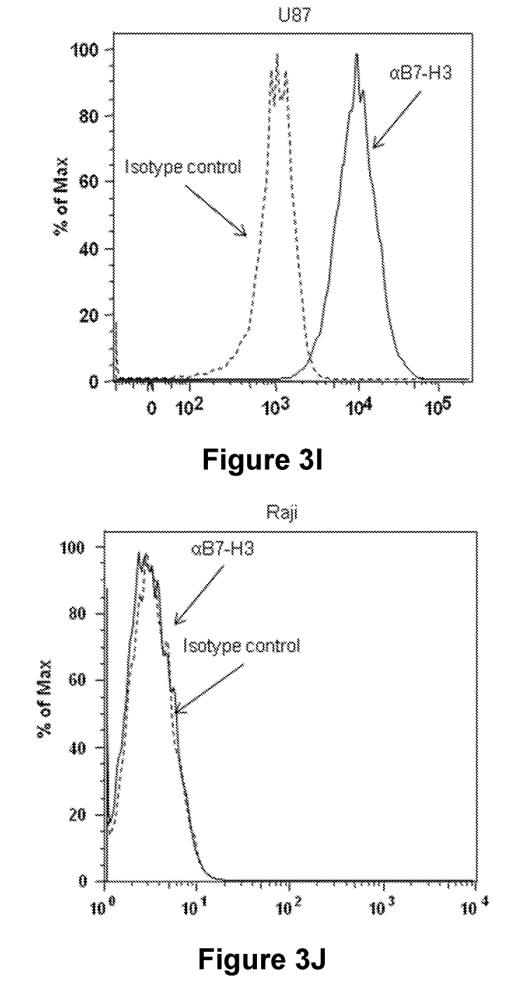

B7-H3 expression was evaluated across a panel of tumor cell lines originated from different human tissue tumor types in order to identify suitable target cell lines for evaluating the biological activity of illustrative B7-H3×CD3 bispecific monovalent Fc diabodies. FIG. 3 shows FACS histograms of anti-B7-H3-PE antibody binding detected on various cancer cell lines. Nine cell lines were confirmed positive for B7-H3 expression and showed a range of B7-H3 expression levels based on the fluorescence intensity of anti-B7-H3-PE antibody binding. The cell lines with the highest B7-H3 expression were: A498 (kidney cancer) (FIG. 3A), JIMT-1/Luc (breast cancer) (FIG. 3B), and A375 (melanoma) (FIG. 3C); medium B7-H3 expression: 22Rv1 (prostate cancer) (FIG. 3D), Detroit562 (pharyngeal cancer) (FIG. 3E), and DU145 (prostate cancer) (FIG. 3F); and low B7-H3 expression: BxPC3 (pancreatic cancer) (FIG. 1G), SKMES-1 (lung cancer) (FIG. 3H), and U87 (glioblastoma) (FIG. 3I). Raji cells, a B-lymphoma cell line that is known to be negative for B7-H3 expression, did not show any fluorescence with the anti-B7-H3-PE antibody used (FIG. 3J). The range of B7-H3 expression on the panel of cell lines evaluated provides a basis to characterize the biological activity of B7-H3 ×CD3 bispecific monovalent Fc diabodies (e.g., DART-A) on tumor cell lines with various levels of target density and derived from different human tissues.

### Example 3

### Binding to B7-H3-Positive Cancer Cell Lines and CD3-Expressing T Cells

The illustrative B7-H3×CD3 bispecific monovalent Fc diabodies were examined for their bispecific binding capacity. Four B7-H3-expressing tumor cell lines (A948, JIMT-1/Luc, Detroit562, and 22Rv1) and human primary T cells were evaluated for DART-A cell surface binding by FACS analysis. Since DART-A binds with CD3, instead of using CD3 as a marker for T cells, the combination of CD4 and CD8 was used as a T cell marker. Therefore, in this study when primary human leukocytes were used, the combined CD4+ plus CD8+ gated events represent the T cell population.

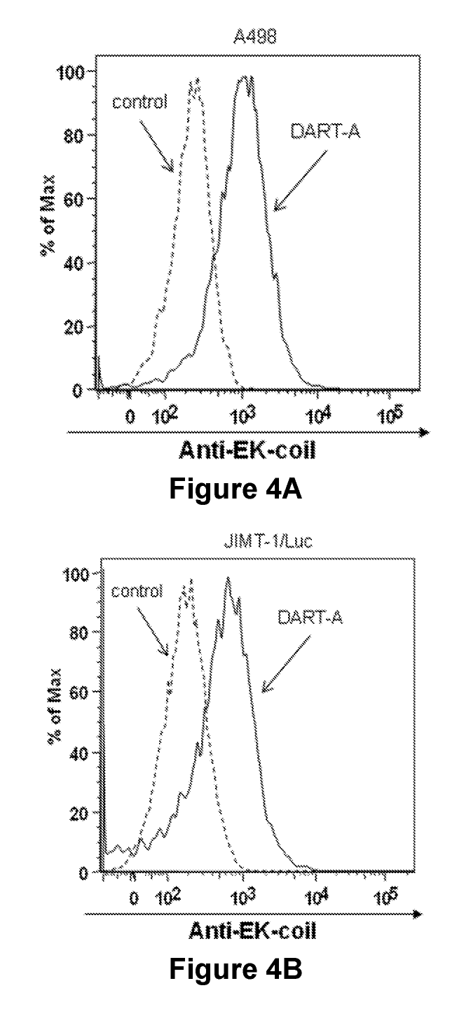

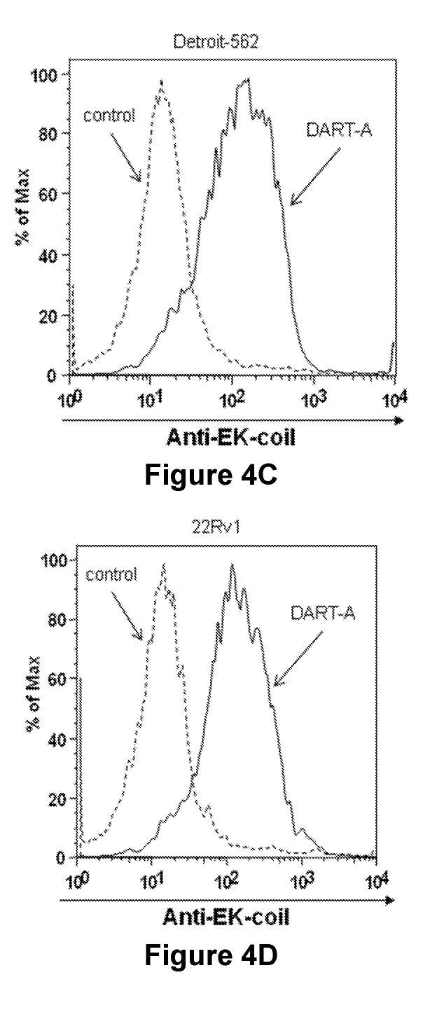

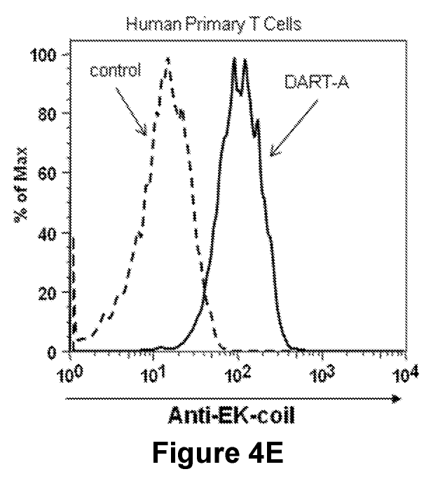

After incubation with 10 μg/mL of DART-A, cell-bound DART-A on target cancer cell lines and T cells was detected using an anti-EK-coil antibody, which recognizes the E-coil/K-coil (EK) Heterodimer-Promoting Domain of the DART-A protein. DART-A showed binding to both human B7-H3-expressing tumor cells (FIGS. 4A-4D) and CD3-expressing T cells (FIG. 4E). DART-B, DART-C, and DART-D were also found to have bispecific binding capacity in similar studies.

### Example 4

### CTL Activity on Multiple Target Cancer Cell Lines with Purified Human T Cells as Effector Cells

After confirming binding to both CD3-expressing T cells and B7-H3-expressing tumor cells (see Example 3), B7-H3×CD3 bispecific monovalent Fc diabody-mediated redirected T cell killing of B7-H3-expressing target cells was evaluated in vitro using 9 human tumor cell lines (A498, JIMT-1/Luc, A375, U87, DU145, BxPC-3, SKMES-1, Detroit562, and 22Rv1) as target cells and normal human T cells as effector cells. Cytotoxicity was determined using the LDH release assay that quantitatively measures the enzymatic activity of LDH, a stable cytosolic enzyme that is released from cells upon cell death. Since the LDH assay measures LDH activity in supernatants from wells containing both target and effector cells, there is a possibility of interference from effector cell death. Therefore, to confirm that the cytotoxicity measured in the LDH release assay was specific to B7-H3×CD3 bispecific monovalent Fc diabody-mediated redirected killing of target cells, cytotoxicity was also evaluated using the luminescence (LUM) assay. In this assay format, the luciferase activity of target cells transfected and selected to stably express the luciferase gene (JIMT-1/Luc cells) is measured and used to enumerate viable target cells remaining at the end of the assay. An FITC×CD3 bispecific monovalent Fc diabody (designated “Control DART”) was used as a control protein in these studies. The FITC×CD3 bispecific monovalent Fc diabody is an anti-fluorescein (FITC)×anti-CD3 diabody protein in which the anti-CD3 binding component is the same as that in the B7-H3×CD3 bispecific monovalent Fc diabodies, but the anti-FITC component represents an irrelevant binding target. Thus, FITC×CD3 bispecific monovalent Fc diabody will engage CD3 on T cells but is not expected to co-engage them with target cells. EC50 values were determined by curve fitting the data to a 3-parameter sigmoidal dose-response function using GraphPad Prism 6 software.

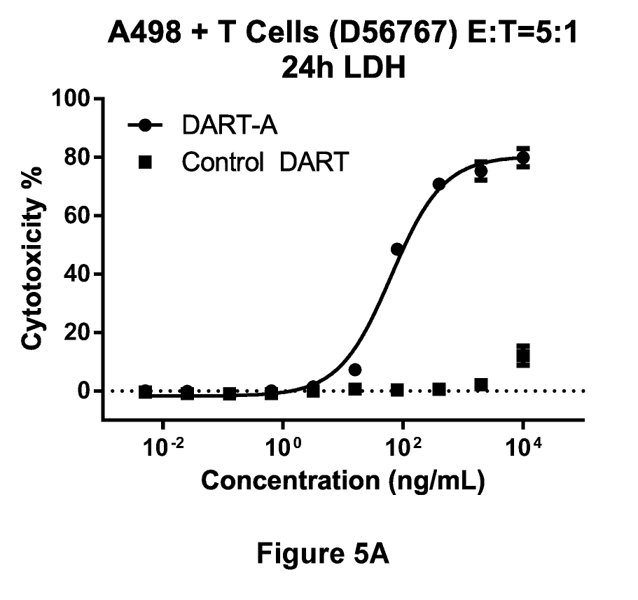

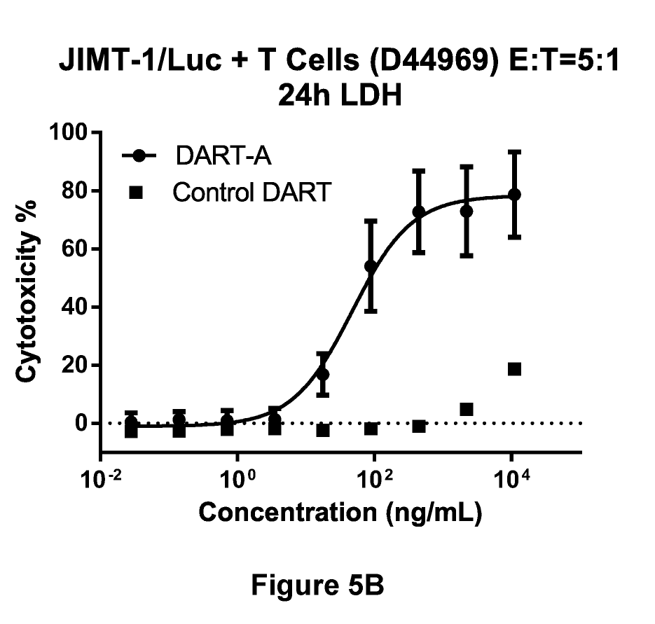

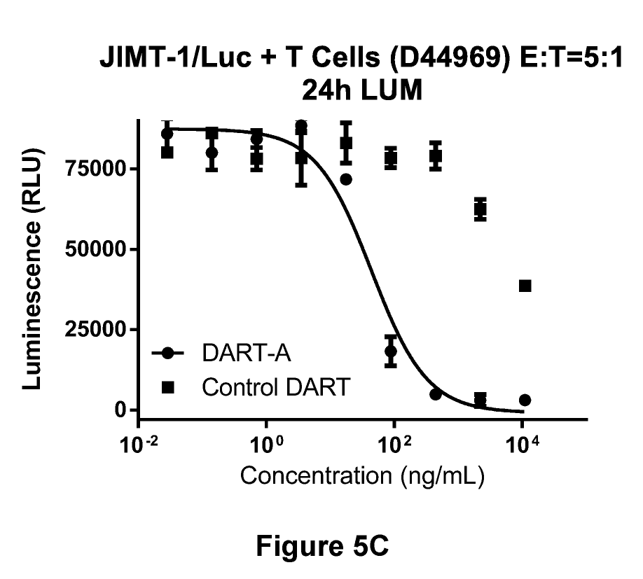

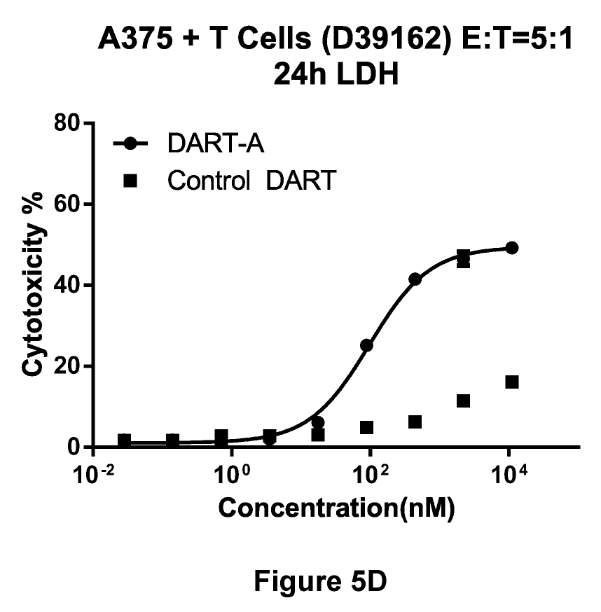

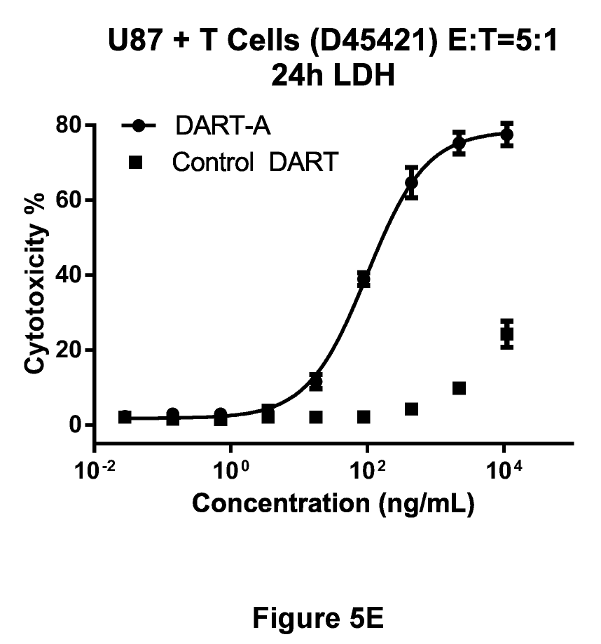

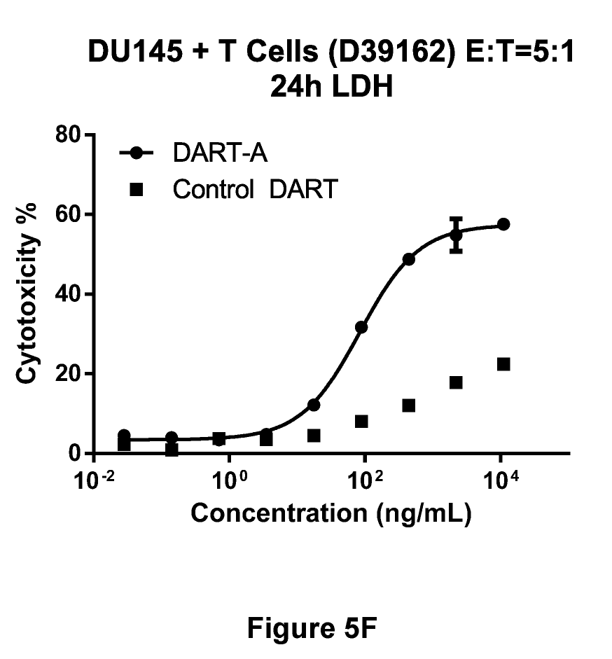

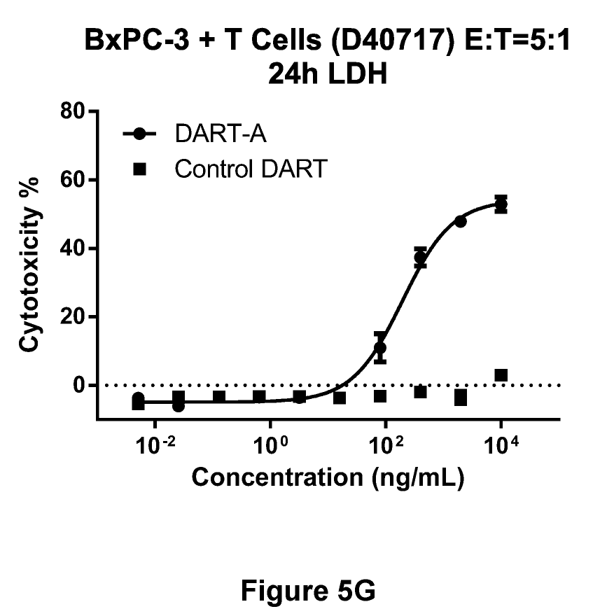

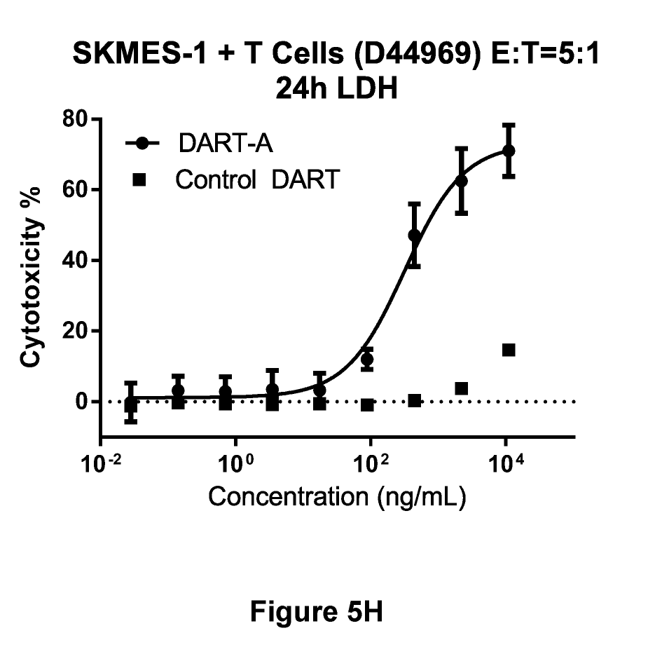

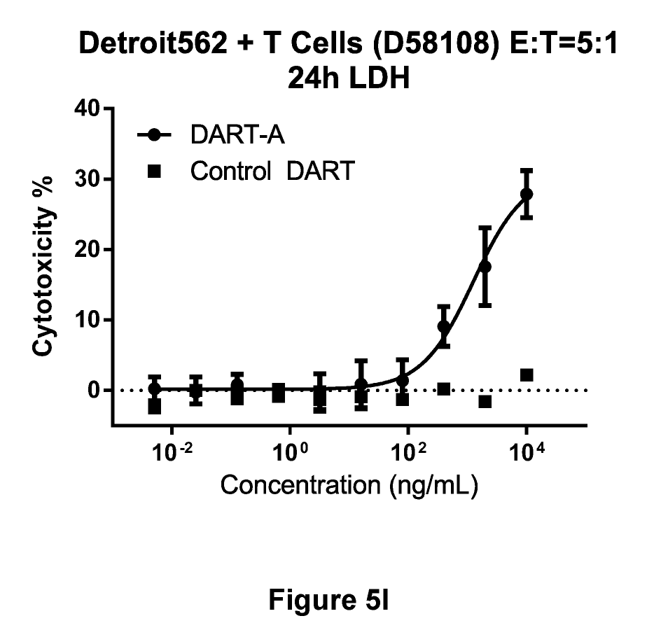

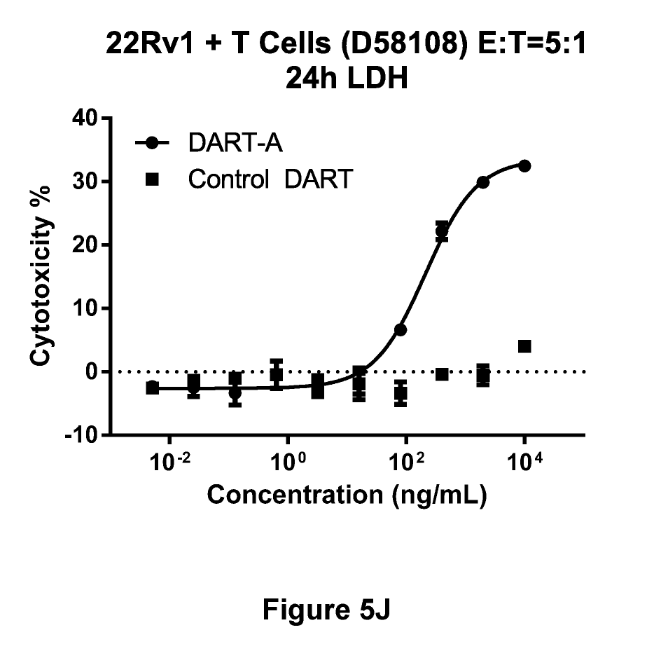

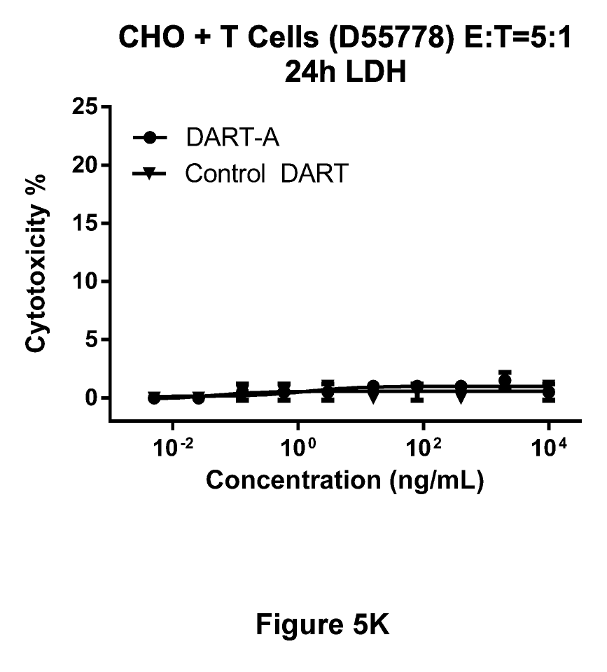

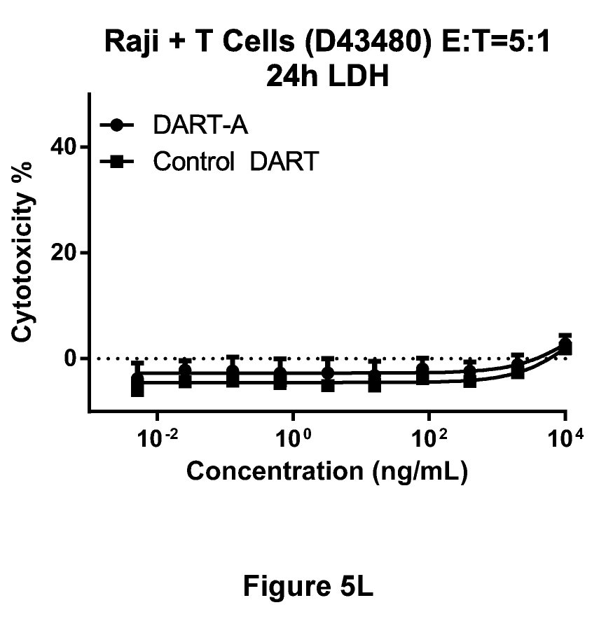

DART-A mediated potent, specific, redirected killing of B7-H3-expressing target cells using purified human T cells as effector cells (E:T cell ratio=5:1). DART-A dose-dependent killing of target cells with representative donor T cells is shown in FIGS. 5A-5J and EC50 values and maximum percent cytotoxicity (Emax) are presented in Table 3. DART-A EC50 concentrations ranging from 47 to 1275 ng/mL were observed across the 9 target cell lines evaluated, with JIMT-1/Luc being the most sensitive cell line (EC50=47 ng/mL). Near complete tumor cell killing was observed using the LUM assay, which specifically measures viable JIMT-1/Luc target cells (see FIG. 5C). DART-A activity was generally correlated with B7-H3 expression, as the lower EC50 values were observed for the target cell lines with higher B7-H3 expression. At the highest concentration evaluated (10,000 ng/mL), minimal or no activity was observed with the Control DART. No cytotoxicity was observed in the presence of DART-A in B7-H3 negative CHO cells (FIG. 5K) or Raji cells (FIG. 5L) confirming the specificity of DART-A activity to B7-H3 expressing target cells.

TABLE 3
Representative Eₘₐₓ and EC₅₀ Concentrations
of DART-A for Redirected Killing of Target
Cell Lines at E:T Cell Ratio of 5:1

| Target Cell | | Eₘₐₓ | EC₅₀ | B7-H3 |
|---|---|---|---|---|
| Line | Tumor type | (%) | (ng/mL) | Expression |
| JIMT-1/Luc | Breast cancer | 78 | 47 | High |
| A498 | Kidney cancer | 80 | 64 | High |
| DU145 | Prostate cancer | 57 | 84 | Medium |
| A375 | Melanoma | 49 | 97 | High |
| U87 | Glioblastoma | 79 | 99 | Low |
| BxPC-3 | Pancreatic cancer | 54 | 196 | Low |
| 22Rv1 | Prostate cancer | 34 | 212 | Medium |
| SKMES-1 | Lung cancer | 73 | 319 | Low |
| Detroit562 | Pharyngeal cancer | 31 | 1275 | Medium |

| | | | |
|---|---|---|---|
| Raji | B-lymphoma | No activity | Negative |
| CHO | Normal Chinese | No activity | Negative |
| | hamster ovary | | |

Similar studies performed with DART-C and DART-D using a number of different target cell lines including A498, and JIMT-1, demonstrated that these two molecules had similar maximum lysis levels (Emax) but were on average 20 times more potent in mediating redirected cell killing than DART-A. DART-B was also tested using a number of different target cell lines including A498, THP-1, and DU145, and was on average 6 times more potent than DART-A.

### Example 5

### CTL Activity of DART-A at Different Effector Cell: Target Cell (E:T) Ratios

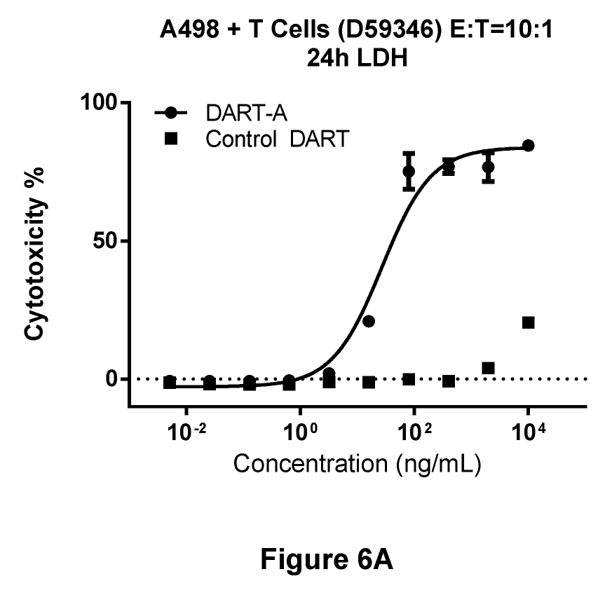

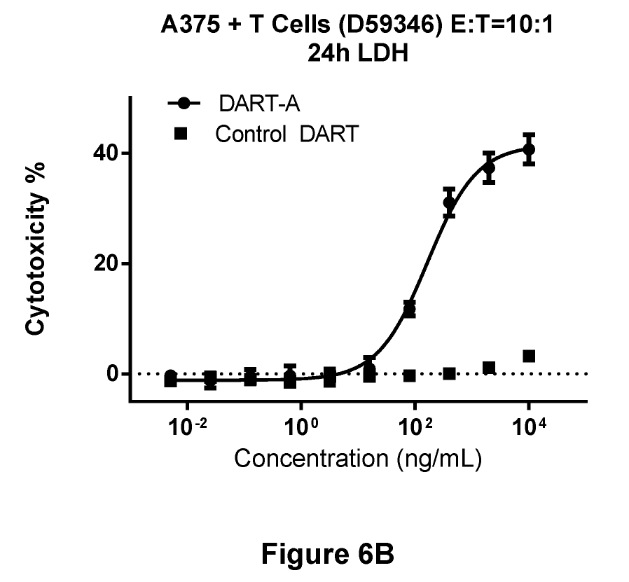

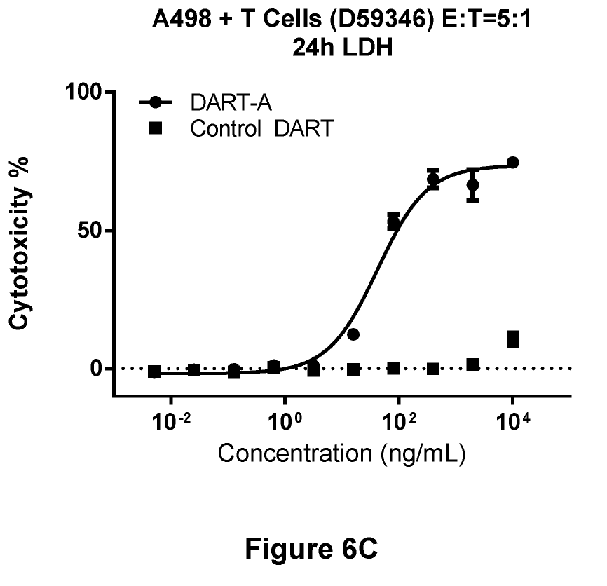

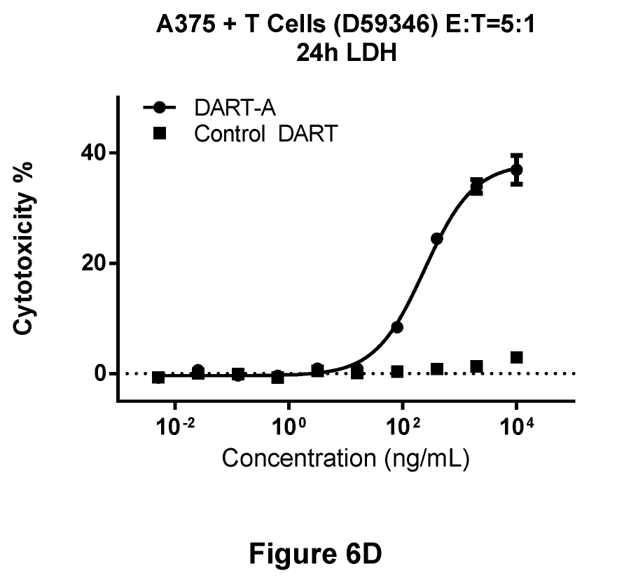

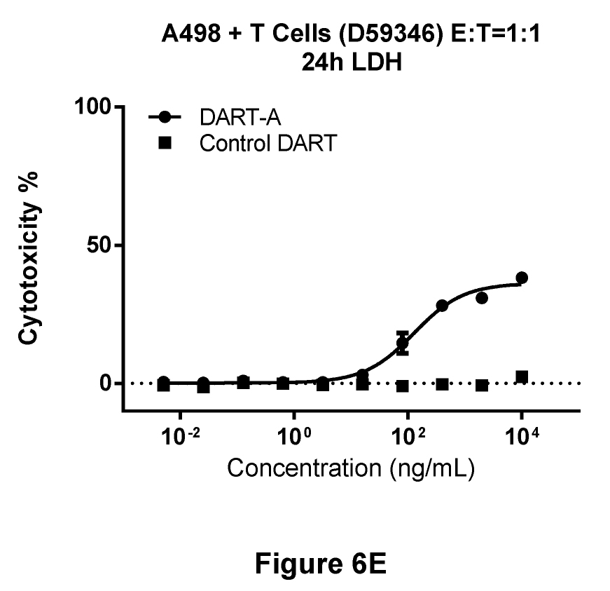

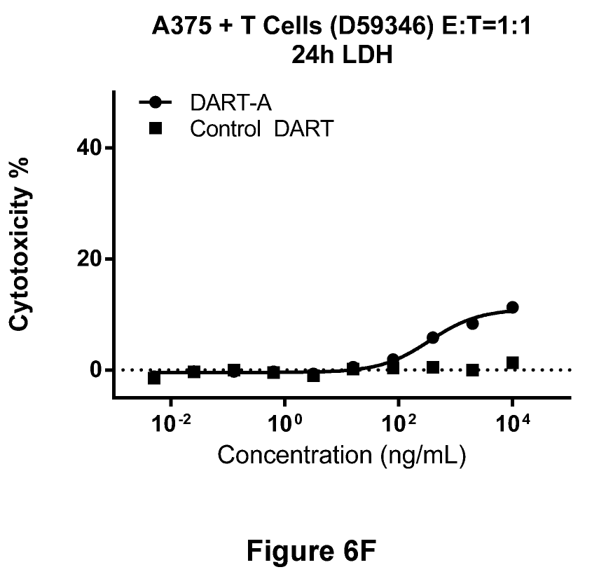

To demonstrate the relationship of E:T cell ratio with B7-H3×CD3 bispecific monovalent Fc diabody-mediated redirected killing, CTL activity of DART-A was further evaluated at E:T cell ratios of 10:1 (FIGS. 6A and 6B), 5:1 (FIGS. 6C and 6D), and 1:1 (FIGS. 6E and 6F) in the LDH assay using A498 (FIGS. 6A, 6C and 6E) and A375 (FIGS. 6B, 6D and 6F) target cells and purified human T cells as effector cells. DART-A showed the highest potency (EC50) and maximum percent cytotoxicity (Emax) at an E:T cell ratio of 10:1 (FIGS. 6A and 6B) and the potency and maximal cytotoxicity decreased with decreasing E:T cell ratios (FIGS. 6A-6F and Table 4). However, specific activity, albeit to a lesser extent, was observed even at the lowest E:T cell ratio evaluated (1:1) (FIGS. 6E and 6F and Table 4).

TABLE 4
Eₘₐₓ and EC₅₀ Concentrations of DART-A for Redirected Killing
of Target Cell Lines at Different E:T Cell Ratios

| | E:T = 10:1 | | E:T = 5:1 | | E:T = 1:1 | |
|---|---|---|---|---|---|---|
| | Eₘₐₓ | EC₅₀ | Eₘₐₓ | EC₅₀ | Eₘₐₓ | EC₅₀ |
| Target Cell Line | (%) | (ng/mL) | (%) | (ng/mL) | (%) | (ng/mL) |
| A498 | 83.91 | 27.2 | 73.56 | 42.51 | 36.3 | 128.5 |
| A375 | 41.5 | 165.6 | 38.1 | 242.7 | 11.01 | 352.5 |

### Example 6

### B7-H3×CD3 Bispecific Monovalent Fc Diabody-Mediated T Cell Activation Depends on Target Cell Engagement

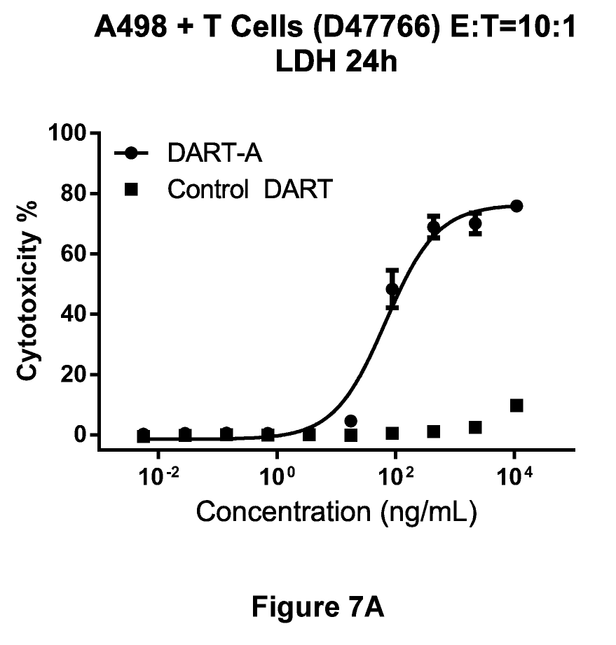

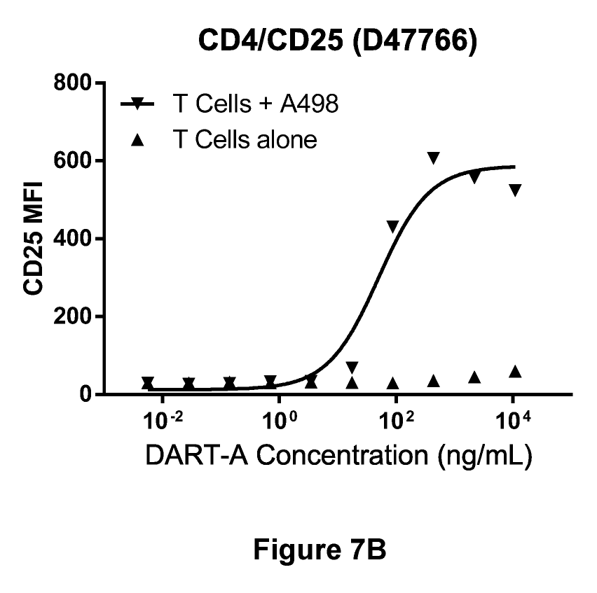

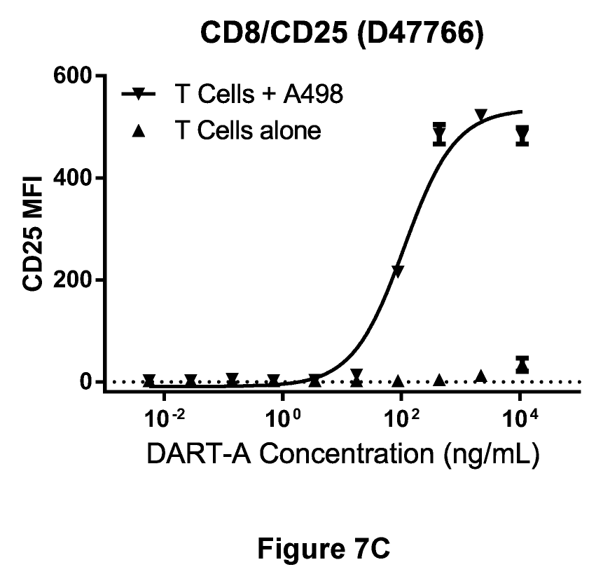

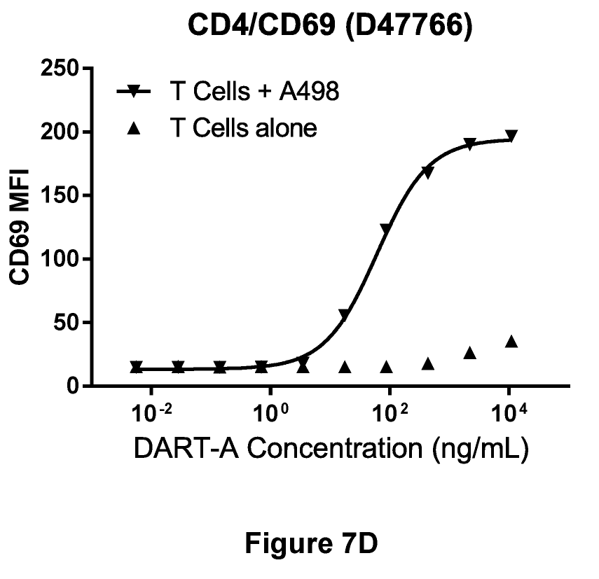

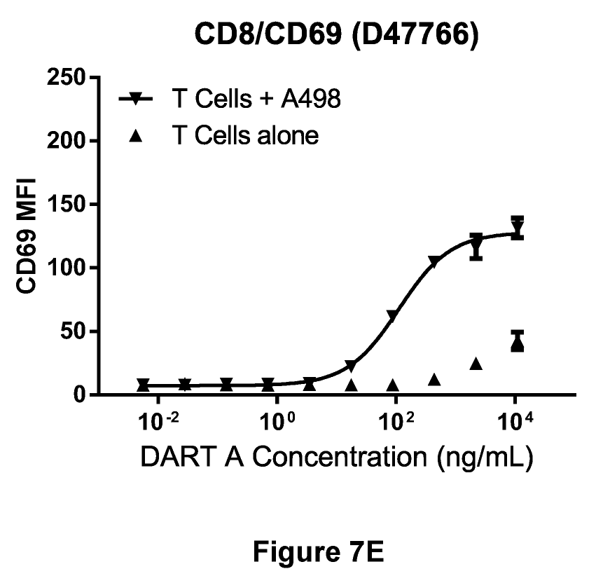

The level of T cell activation induced by B7-H3×CD3 bispecific monovalent Fc diabodies was evaluated in human T cells either alone or in the presence of B7-H3-expressing target cells (A498) at an E:T cell ratio of 10:1. The results of these studies using DART-A are shown in FIG. 7. Flow cytometry analyses revealed upregulation of CD25 (FIGS. 7B and 7C) and CD69 (FIGS. 7D and 7E), T cell activation markers, on CD4+(FIGS. 7B and 7D) and CD8+(FIGS. 7C and 7E) T cell subsets in a dose-dependent manner by DART-A in the presence of B7-H3-expressing target cells (FIGS. 7B-7D). DART-A-mediated T cell activation correlated with the cytotoxicity of target cells (FIG. 7A).

At all concentrations evaluated using either DART-A or the Control DART, no T cell activation was observed with T cells alone in the absence of B7-H3-expressing target cells (FIGS. 7B-7E). These data suggest that T cell activation mediated by B7-H3×CD3 bispecific monovalent Fc diabodies such as DART-A is dependent upon effector cell-target cell co engagement. Furthermore, no cytotoxicity was observed when T cells alone were incubated with DART-A (FIGS. 7B-7E) or the Control DART in the CTL assay. In contrast, significant DART-A-mediated cytoxicity was observed in the presence of target cells (FIG. 7A). Comparable results were found in similar studies performed using DART-B, DART-C or DART-D.

### Example 7

### B7-H3×CD3 Bispecific Monovalent Fc Diabody-Mediated T Cell Proliferation Upon Target Cell Co-Engagement

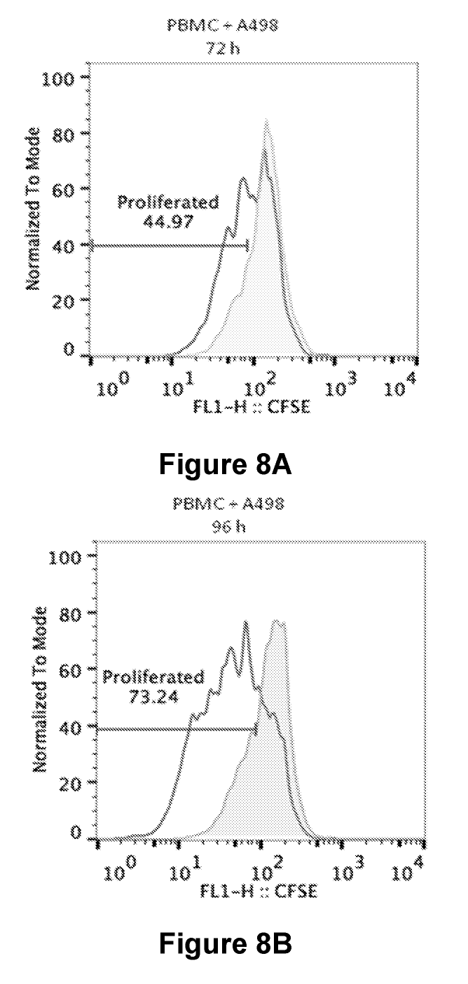

T cell expansion associated with CTL activity induced by a bispecific antibody has been previously reported (Klinger M et al. (2012) “Immunopharmacologic Response Of Patients With B-Lineage Acute Lymphoblastic Leukemia To Continuous Infusion Of T Cell-Engaging CD19/CD3-Bispecific BiTE Antibody Blinatumomab,” Blood 119(26):6226-6233). Therefore, after observing that treatment with B7-H3×CD3 bispecific monovalent Fc diabodies resulted in dose-dependent depletion of target cells accompanied by an increase in T cell activation markers, the expansion of T cells cultured with target cells and B7-H3×CD3 bispecific monovalent Fc diabodies was evaluated. To do this, human PBMCs were labeled with CFSE and co-cultured with A498 target cells at an E:T cell ratio of 10:1 in the presence of DART-A or Control DART at a concentration of 10 μg/mL for 72 or 96 hours. As each cell division reduces the level of dye contained in the daughter cells by half, proliferation of CFSE-labeled T cells was monitored by measuring levels of CFSE over time by FACS analysis. FIG. 8A (72 hours) and FIG. 8B (96 hours) show the CFSE-staining profiles following incubation after starting the co culture in the presence of DART-A or Control DART and target cells. The presence of DART-A led to proliferation of CFSE-labeled T cells with the percentages of proliferation around 45% after 72 hours (FIG. 8A) and 73% after 96 hours (FIG. 8B) incubation. In contrast, no proliferation of CFSE-labeled T cells was observed in the presence of the Control DART (FIGS. 8A and 8B).

### Example 8

### Characterization of Binding to Human and Cynomolgus Monkey B7-H3-Expressing CHO Cells and Redirected Killing

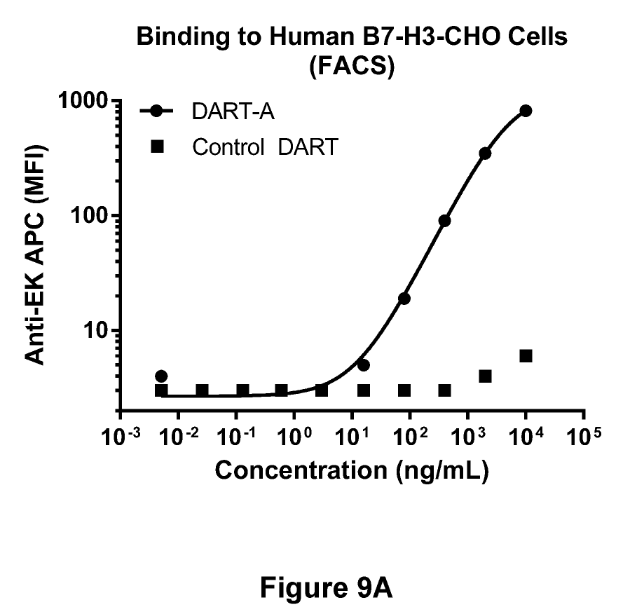

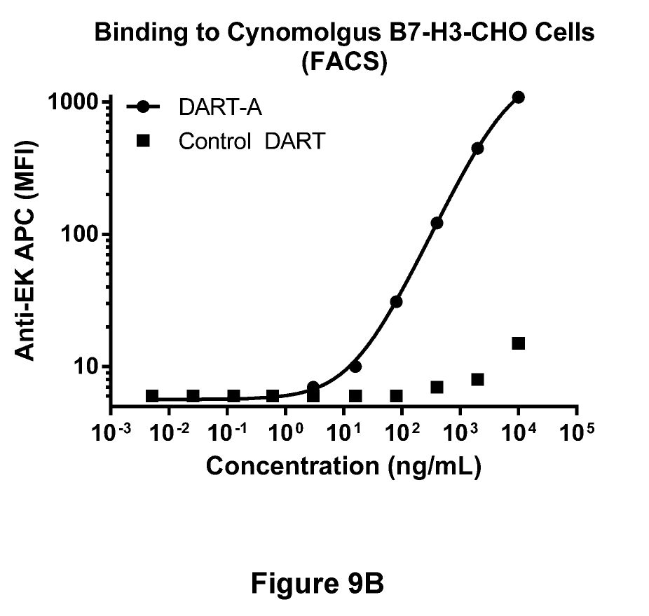

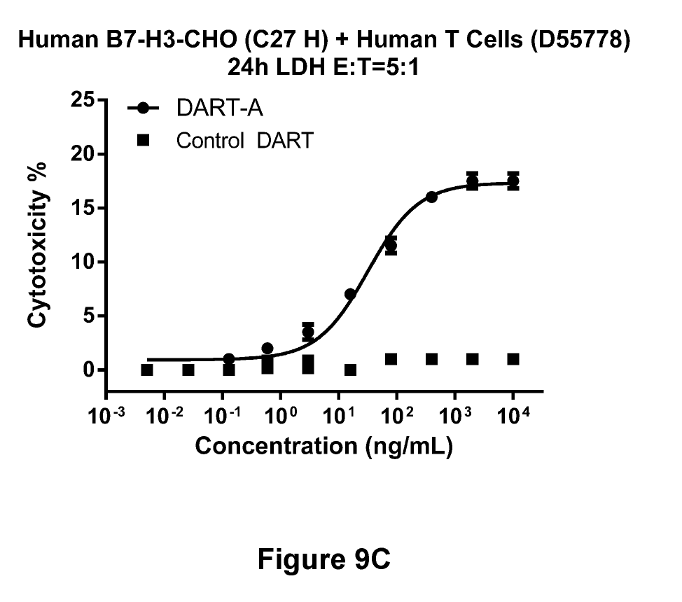

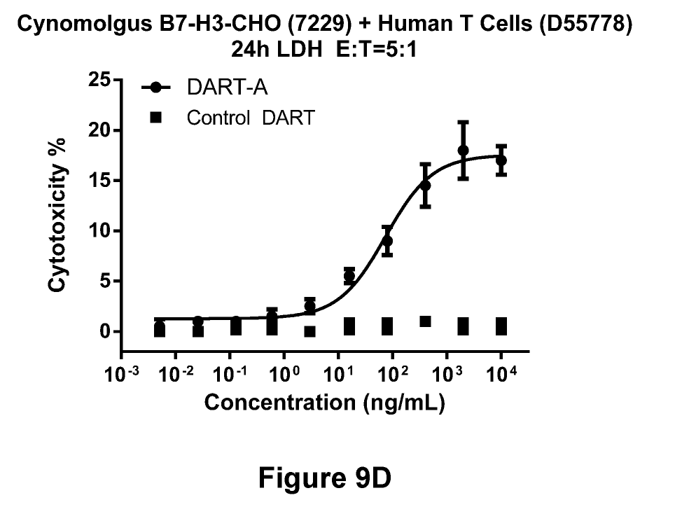

To confirm the capability of DART-A to cross-react with cynomolgus monkey B7-H3, DART-A binding to CHO cells transfected with human B7-H3 (huB7-H3-CHO) (FIG. 9A) or cynomolgus monkey B7-H3 (cyno-B7-H3-CHO) (FIG. 9B) was evaluated by flow cytometry. Cells were treated with 5-fold decreasing concentrations of DART-A or Control DART protein starting from 10 μg/mL and cell-bound DART-A was detected using the anti-EK-coil antibody. FIGS. 9A and 9B shows concentration-dependent binding of DART-A to both human B7-H3-expressing and cynomolgus monkey B7-H3-expressing CHO cells, respectively.

To evaluate B7-H3×CD3 bispecific monovalent Fc diabody-mediated redirected killing using CHO cells expressing human B7-H3 or cynomolgus monkey B7-H3, cells were incubated with varying concentrations of DART-A in the presence of human T cells at an E:T cell ratio of 5:1. As shown in FIGS. 9C and 9D, efficient DART-A-mediated killing was observed for both cynomolgus monkey (FIG. 9C) and human (FIG. 9D) B7-H3-expressing CHO cells.

### Example 9

### B7-H3 ×CD3 Bispecific Monovalent Fc Diabody-Mediated Redirected Killing of Target Cells with Cynomolgus Monkey PBMCs as Effector Cells

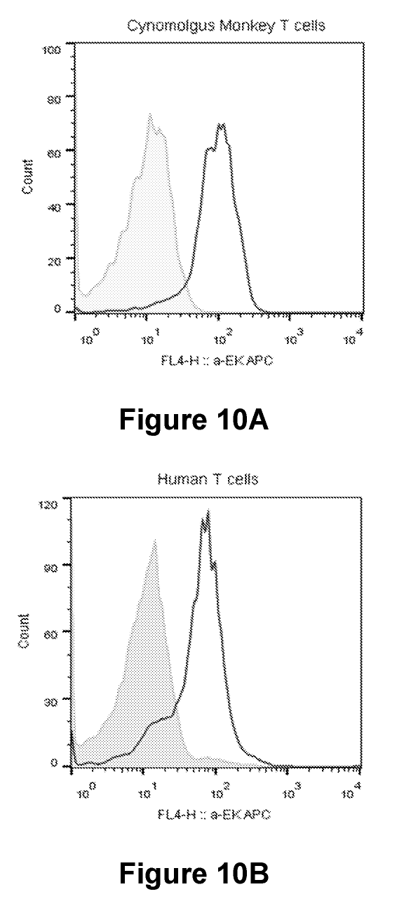

DART-A binding to cynomolgus monkey T cells was assessed by flow cytometry where the binding of DART-A was profiled in gated CD4+ and CD8+ T cell populations. DART-A binding to cynomolgus monkey T cells (FIG. 10A) was similar to human T cells (FIG. 10B).

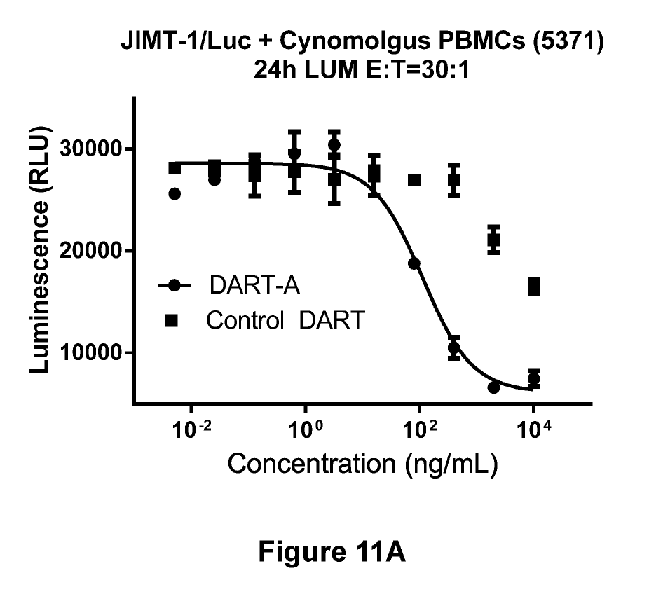

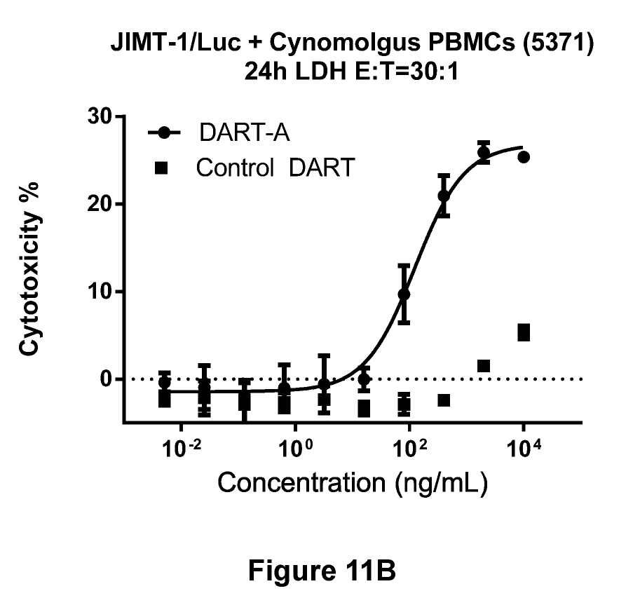

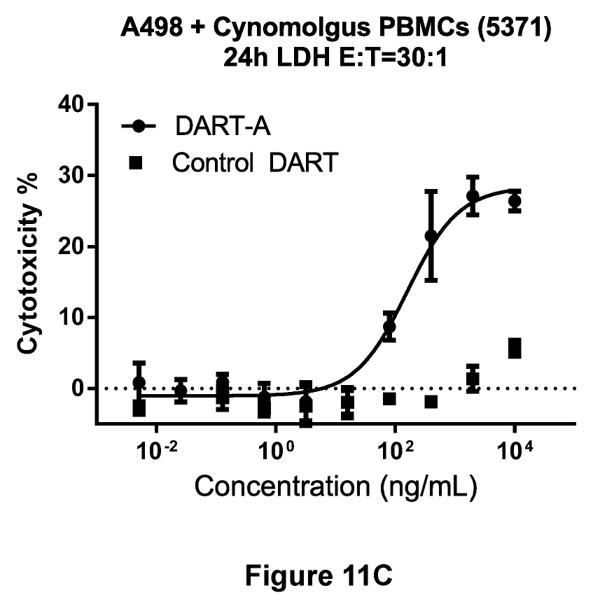

After confirming the cross-reactivity of DART-A with cynomolgus monkey T cells, DART-A-mediated ex vivo CTL activity was evaluated using cynomolgus monkey PBMCs. DART-A or Control DART was added to cynomolgus monkey PBMCs mixed with B7-H3-expressing target cells (JIMT-1/Luc (FIGS. 11A and 11B) or A498 (FIG. 11C)) at an E:T cell ratio of 30:1 and incubated for 24 hours. As shown in FIGS. 11A-11C, dose-dependent DART-A-mediated ex vivo cytotoxicity was observed using cynomolgus monkey PBMCs as effector cells against human B7-H3-expressing target cell lines.

### Example 10

### Co-Mix Xenograft Model of 22Rv1 Human Prostate Cancer

Human T cells were isolated from heparinized whole blood according to the manufacturer's protocol provided in the RosetteSep T cell isolation kit (STEMCELL Technologies, Vancouver, Canada). The purified T cells were subsequently activated by exposing the cells to anti-CD3 (OKT-3; 1 μg/mL) and anti-CD28 (66 μg/mL) antibodies or to anti-CD3/CD28 Dynabeads (1:1 ratio) for a period of 48 hours. Following stimulation, the cells were grown in RPMI 1640 medium with 10% FBS and 1% penicillin/streptomycin in the presence of IL2 (7.6 ng/mL) for up to 3 weeks.

Human T cells (1×10⁶) and 22Rv1 tumor cells (5×10⁶) were combined and injected subcutaneously (SC) on Day 0 after being resuspended in 200 μL of Ham's F12 medium. Following 22Rv1 tumor cell and T cell implantation, mice were treated IV with vehicle control (●), Control DART (0.5 mg/kg) (∘), or DART-A at 4 different dose levels (0.004 (♦), 0.02 (▾), 0.1 (▴), or 0.5 (▪) mg/kg) once daily for 4 days starting on the day of tumor cell implantation (Days 0, 1, 2, and 3).

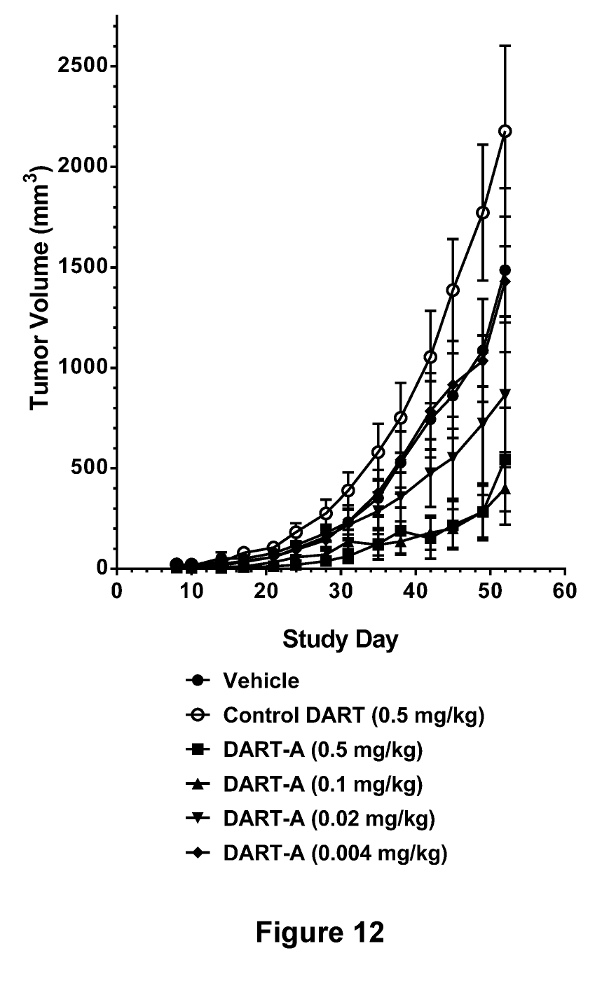

As shown in FIG. 12, the growth of 22Rv1 tumor cells was delayed and inhibited following IV treatment with DART-A once daily on Days 0 to 3 at a dose level of 0.1 (▴) or 0.5 (▪) mg/kg. While there was partial inhibition in tumor growth at the 0.02 mg/kg (▾) DART-A dose level, it did not reach significance. No inhibition in tumor growth was noted at the 0.004 mg/kg (♦) DART-A dose level.

### Example 11

### Co-Mix Xenograft Model of A498 Human Kidney Cancer

Human T cells were isolated and prepared as above. Human T cells (1×10⁶) and A498 tumor cells (5×10⁶) were combined and injected SC on Day 0 after being resuspended in 200 μL of Ham's F12 medium. Following A498 tumor cell and T cell implantation, mice were treated IV with vehicle control (●), Control DART (0.5 mg/kg) (∘), or DART-A at 4 different dose levels (0.004 (♦), 0.02 (▾), 0.1 (▴), or 0.5 (▪) mg/kg) once daily for 4 days starting on the day of tumor cell implantation (Days 0, 1, 2 and 3).

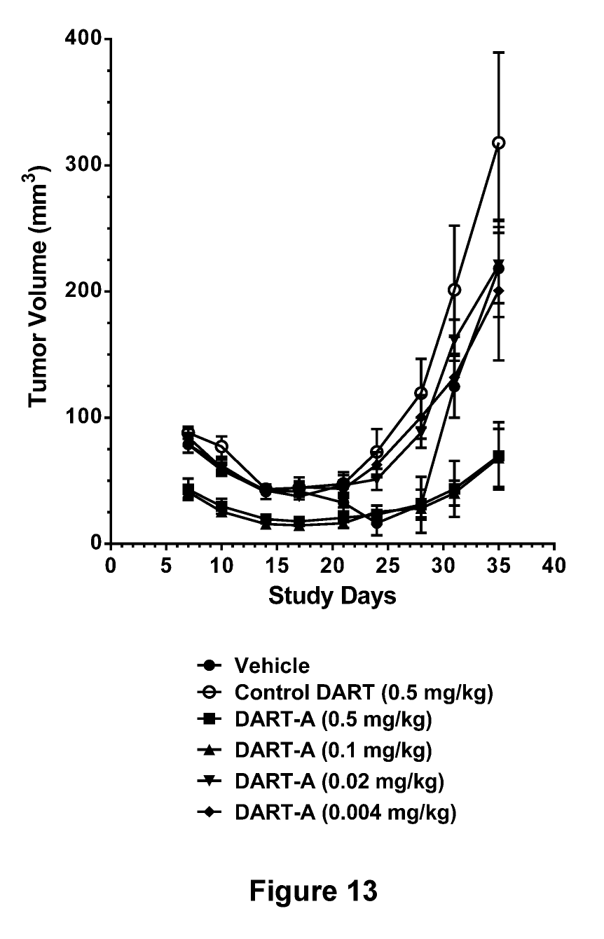

As shown in FIG. 13, A498 tumor cells injected on Day 0 in the presence of activated human T cells showed some initial tumor shrinkage even in control animals, the result of tumor adaptation to the in vivo environment. Brisk tumor growth, however, was noted at later time points in animals receiving vehicle control (●) and Control DART (∘) as well as in animals treated once daily on Days 0, 1, 2 and 3 with 0.004 (♦) or 0.02 (▾) mg/kg DART-A, with no inhibition in tumor growth compared to control animals. Tumor growth was delayed and inhibited following IV treatment with DART-A at a dose level of 0.1 (▴) or 0.5 (▪) mg/kg.

### Example 12

### Established A498 Human Kidney Cancer Model in Human PBMC-Reconstituted Mice

To evaluate the activity of DART-A on established tumor xenografts in NSG B2m−/− mice, a human effector cell-reconstituted model was employed. This model also provides an environment in which the anti-tumor activity depends on the recruitment of engrafted human T cells by DART-A to the established tumor.

Female NSG B2m−/− mice (Jackson Laboratory) were used for the established tumor studies (n=7-8/group). beta-2 microglobulin (B2m) knockout mice with impaired expression of MHC class I were employed in order to delay and minimize the incidence and severity of graft-versus-host disease (GVHD) associated with the engraftment of human peripheral blood mononuclear cells (PBMCs). It should be noted, however, that the lack of B2m expression in these mice is also associated with impaired expression of FcRn (Israel, E. J. et al. (1996) “Increased clearance of IgG in mice that lack beta 2-microglobulin: possible protective role of FcRn,” Immunology 89: 573-578). Hence, the half-life of DART-A in these mice was expected to be short compared to that in wild-type mice or primates. Based on previous studies in this strain of mice with molecules comprising a human Fc Region, a dosing frequency of once every 2 to 4 days was adopted to ensure adequate exposure in these studies.

Human PBMCs were isolated from heparinized whole blood using Ficoll-Paque according to the manufacturer's protocol. A498 tumor cells (5×10⁶ viable cells) were resuspended in 100 μL of Ham's F12 and injected intradermally (ID) on Day 0, followed by the intraperitoneal (IP) injection (200 μL, saline) of human PBMCs (1×10⁷ viable cells) on Day 13. The timing of PBMC inoculation with respect to tumor cell implantation related to the growth rate of the tumor cells and was empirically determined in order to obtain optimal human effector cell reconstitution with tumor sizes of approximately 150-300 mm³ at the time of randomization and treatment initiation. The treatment period was on Days 33, 35, 36, 39, 41, 43, 46, 48 and 50 for a total of 9 doses administered IV including vehicle control, Control DART (1 mg/kg), or DART-A at 4 different dose levels (0.001, 0.01, 0.1, or 1 mg/kg).

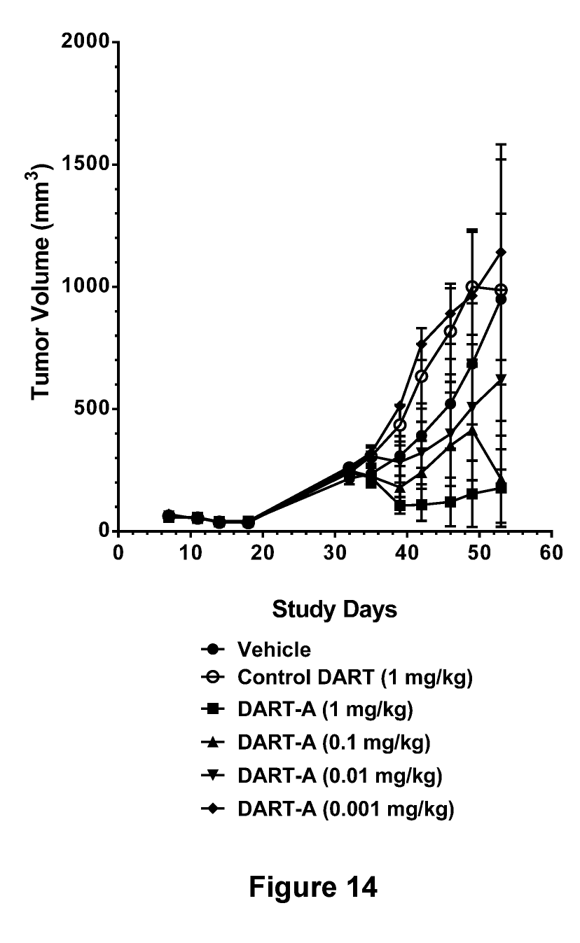

A498 tumors had reached an average approximate volume of 250 mm³ on Day 32 prior to treatment initiation (FIG. 14). In the group that received 1 mg/kg (▪) DART-A, tumor volume regressed from 242±19 mm³ on Day 32 to 106±35 mm³ by Day 39. In the group that received 0.1 mg/kg (▴) DART-A, there was a smaller reduction in tumor volume (249±25 to 181±87 mm³), while in the 0.01 mg/kg (▾) group there was a period of cytostasis during the same interval (Days 32 to 39). In the 1 mg/kg (▪) DART-A group, tumor growth continued to regress in 5/7 animals, but relapsed in the remaining 2 animals by the end of the study (Day 53). By the end of the study in the 0.1 (▴) and 0.01 (▾) mg/kg groups, tumor growth remained regressed in 5/7 and 4/7 animals, respectively. Progression of tumor growth was evident for all animals treated with 0.001 (♦) mg/kg DART-A. No effect on the growth of the tumors was noted with vehicle control (●) or the Control DART (∘).

A498 human kidney cancer model in human PBMC-reconstituted mice was also used to evaluate the activity of DART-B dosed at 0.02, 0.1 or 0.5 mg/kg, Control DART (0.5 mg/kg), or vehicle control. Animals treated with DART-B showed substantial inhibition of tumor growth at all doses, while no effect on the growth of the tumors was noted with vehicle control or the Control DART.

### Example 13

### Established Detroit562 Human Pharyngeal Cancer Model in Human PBMC-Reconstituted Mice

Human PBMCs were prepared as above. Detroit562 tumor cells (5×10⁶ viable cells) were resuspended in 100 μL of Ham's F12 and injected ID on Day 0. Human PBMCs (1×10⁷ viable cells) were implanted by IP injection (200 saline) on Day −1, one day prior to tumor cell implantation, in NSG B2m−/− mice. The treatment period was on Days 20, 22, 23, 26, 28, 30, 33, 35 and 37 for a total of 9 doses administered IV and included vehicle control, Control DART (0.5 mg/kg), or DART-A at 4 different dose levels (0.1, 0.25, 0.5, or 1 mg/kg).

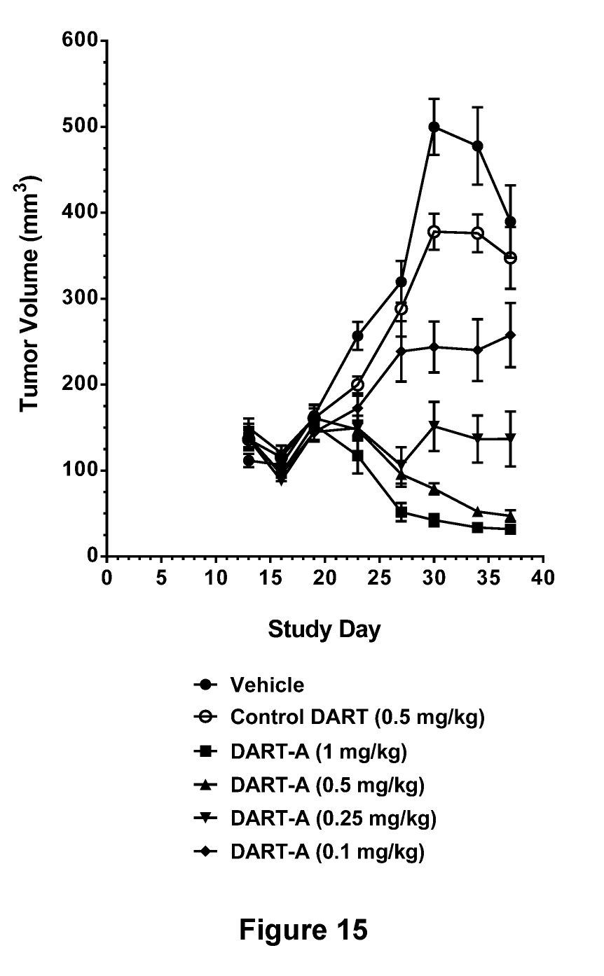

Detroit562 tumors had reached a tumor volume of approximately 150 mm³ on Day 19 prior to treatment initiation (FIG. 15). The tumors in the groups that received DART-A at the 1 (▪), 0.5 (▴), and 0.25 (▾) mg/kg dose levels decreased in size during the treatment period with the nadir being reached on Day 27 (106±21 mm³) for the 0.25 (▾) mg/kg group (FIG. 15). For the 1 (▪) and 0.5 (▴) mg/kg groups, the greatest tumor reduction was observed on Day 37, the last day of the study, with mean tumor volumes of 32±6 and 47±7 mm³, respectively (FIG. 15). In the 0.1 (♦) mg/kg DART-A group, reduced tumor growth was observed with a maximum tumor volume (258±37 mm³) that was reduced compared with that of the Control DART (∘) (347±36 mm³) and vehicle (390±42 mm³) groups at the end of the study (Day 37) (FIG. 15).

### Example 14

### Established Detroit562 Human Pharyngeal Cancer Model in Human PBMC-Reconstituted Mice Dosing Study

To evaluate the activity of DART-A on established tumor xenografts in MHCl1 −/− mice, a human effector cell-reconstituted model was employed. This model also provides an environment in which the anti-tumor activity depends on the recruitment of engrafted human T cells by DART-A to the established tumor. In these studies, weekly and bi-weekly (i.e., every other week) dosing regimens were examined.

Human PBMCs were prepared as described above. Detroit562 tumor cells (5×10⁶ viable cells) were re-suspended in 50 μL of Ham's F12 medium and combined with 50 μL of Matrigel, and then injected intradermally (ID) on Day 0. Human PBMCs (1×10⁷ viable cells) were implanted by IP injection (200 Ham's F12 medium) on Day 0, in MHCl1 −/− mice. The treatment period was initiated on Day 15. Group I mice were administered DART-A (0.5 mg/kg) once per week (Q1W) on days 15, 22, 29, 36 and 43 for a total of 5 doses administered IV. Group II mice were administered DART-A (0.5 mg/kg) once every two weeks (Q2W) on days 15, 29, and 43 for a total of 3 doses, administered IV. The vehicle control animals were dosed once per week.

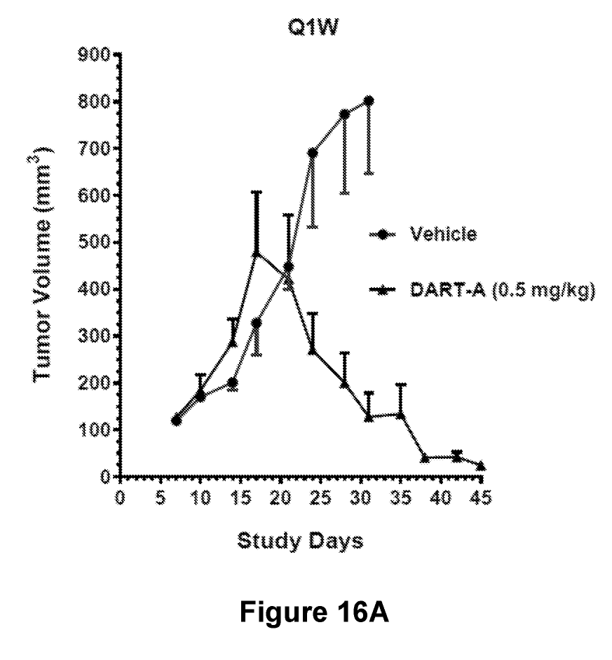

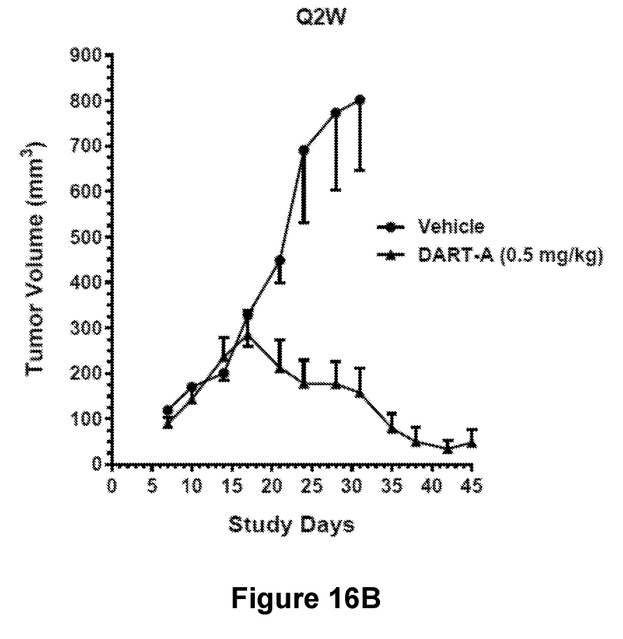

Detroit562 tumors ranged from 200.49±15.58 mm³ to 287.5±48.79 mm³ on Day 14 prior to treatment initiation. The tumors in DART-A-treated animals in both Groups I and II (▴) decreased in size during the treatment period as compared to vehicle treated animals (●) (FIGS. 16A and 16B). In Group I, DART-A-treated animals, reduced tumor growth was observed with a [maximum] tumor volume (24.3±9.5 mm³) on Day 45 that was reduced compared with that of the animals treated with vehicle (801.9±155.5 mm³) on Day 31 (FIG. 16A). In Group II, DART-A-treated animals, reduced tumor growth was observed with a [maximum] tumor volume (47.6±28.5 mm³) that was reduced compared with that of the animals treated with vehicle (801.9±155.5 mm³) on Day 31 (FIG. 16B).

### Example 15

### Toxicokinetic Studies in Cynomolgus Monkeys

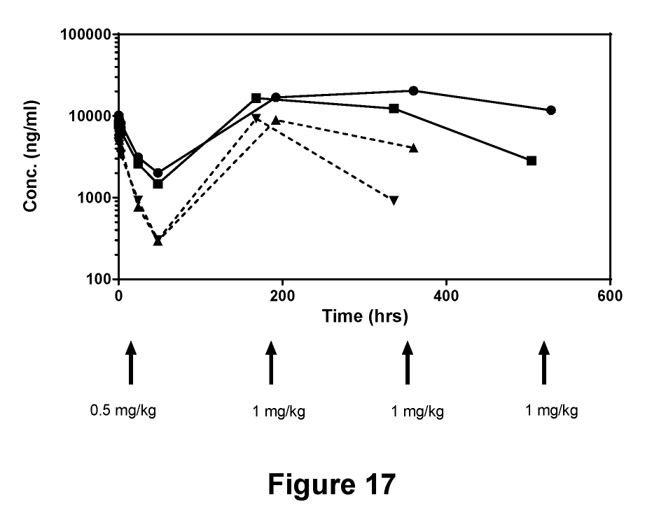

Three non-GLP, pilot, exploratory, dosing studies of DART-A, DART-B, DART-C, or DART-D were performed in cynomolgus monkeys. In one dose-escalation study two groups of cynomolgus monkeys (n=2/group; 1M/1F) were administered one dose of 0.5 mg/kg then three weekly doses of 1.0 mg/kg of DART-A or DART-B. There were no significant findings for either group, although transient increases in IL-6 levels were observed following infusion. There were no apparent clinical observations associated with increased cytokine levels. The serum concentrations of DART-A and DART-B were examined during the course of the study (FIG. 17). As shown in FIG. 17 both molecules exhibited acceptable pharmacokinetic profiles. The serum levels of DART-A showed less variability over the course of the study.

In a single dose study two groups of cynomolgus monkeys (n=2/group; 1M/1F) were administered one dose of 0.5 mg/kg of DART-D or one dose of 0.5 mg/kg then three weekly doses of 1.0 mg/kg of the Control DART. Both animals treated with DART-D at 0.5 mg/kg exhibited abnormal clinical signals. In a further study four groups of cynomolgus monkeys (n=2/group; 1M/1F) were administered four weekly doses of either 0.075, 0.15 or 0.3 mg/kg of DART-D, or four weekly doses of 0.5 mg/kg of DART-C. In these studies all the doses were tolerated. However, there was an apparent transient effect on platelet levels at 0.15 and 0.3 mg/kg of DART-D, and at 0.5 mg/kg of DART-C.

All publications and patents mentioned in this specification are herein incorporated by reference to the same extent as if each individual publication or patent application was specifically and individually indicated to be incorporated by reference in its entirety. While the invention has been described in connection with specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth.

What is claimed is:

1. A B7-H3×CD3 bispecific monovalent Fc diabody capable of specific binding to B7-H3 and to CD3, wherein the diabody comprises a first, a second and a third polypeptide chain, wherein said polypeptide chains form a covalently bonded complex, and wherein:
(A) said first polypeptide chain has the amino acid sequence of SEQ ID NO:53;
(B) said second polypeptide chain has the amino acid sequence of SEQ ID NO:55; and
(C) said third polypeptide chain has the amino acid sequence of SEQ ID NO:57.

2. A pharmaceutical composition comprising the B7-H3×CD3 bispecific monovalent Fc diabody of claim 1 and a physiologically acceptable carrier.

3. Use of the B7-H3×CD3 bispecific monovalent Fc diabody of claim 1 or the pharmaceutical composition of claim 2 in the treatment of a disease or condition associated with or characterized by the expression of B7-H3.

4. The use of claim 3, wherein said disease or condition associated with or characterized by the expression of B7-H3 is cancer.

5. The use of claim 4, wherein said cancer is selected from the group consisting of: an acute myeloid leukemia, an adrenal gland tumor, an AIDS-associated cancer, an alveolar soft part sarcoma, an astrocytic tumor, bladder cancer, bone cancer, a brain and spinal cord cancer, a metastatic brain tumor, a breast cancer, a carotid body tumors, a cervical cancer, a chondrosarcoma, a chordoma, a chromophobe renal cell carcinoma, a clear cell carcinoma, a colon cancer, a colorectal cancer, a cutaneous benign fibrous histiocytoma, a desmoplastic small round cell tumor, an ependymoma, a Ewing's tumor, an extraskeletal myxoid chondrosarcoma, a fibrogenesis imperfecta ossium, a fibrous dysplasia of the bone, a gallbladder or bile duct cancer, gastric cancer, a gestational trophoblastic disease, a germ cell tumor, a head and neck cancer, hepatocellular carcinoma, a glioblastoma, an islet cell tumor, a Kaposi's Sarcoma, a kidney cancer, a leukemia, a lipoma/benign lipomatous tumor, a liposarcoma/malignant lipomatous tumor, a liver cancer, a lymphoma, a lung cancer, a medulloblastoma, a melanoma, a meningioma, a malignant mesothelioma, a multiple endocrine neoplasia, a multiple myeloma, a myelodysplastic syndrome, a neuroblastoma, a neuroendocrine tumors, a non-small cell lung cancer, an ovarian cancer, a pancreatic cancer, a pharyngeal cancer, a papillary thyroid carcinoma, a parathyroid tumor, a pediatric cancer, a peripheral nerve sheath tumor, a phaeochromocytoma, a pituitary tumor, a prostate cancer, a posterious uveal melanoma, a rare hematologic disorder, a renal cell carcinoma, a renal metastatic cancer, a rhabdoid tumor, a rhabdomysarcoma, a sarcoma, a skin cancer, a soft-tissue sarcoma, a squamous cell cancer, a stomach cancer, a synovial sarcoma, a testicular cancer, a thymic carcinoma, a thymoma, a thyroid metastatic cancer, and a uterine cancer.

6. The use of claim 5, wherein said cancer is selected from the group consisting: bladder cancer, breast cancer, colorectal cancer, gastric cancer, glioblastoma, kidney cancer, lung cancer, melanoma, neuroblastoma, ovarian cancer, pancreatic cancer, pharyngeal cancer, prostate cancer, renal cell carcinoma, rhabdomyosarcoma, and squamous cell cancer of the head and neck (SCCHN).

7. A B7-H3×CD3 bispecific monovalent Fc diabody capable of specific binding to B7-H3 and to CD3, wherein the diabody comprises a first, a second and a third polypeptide chain, wherein said polypeptide chains form a covalently bonded complex, and wherein:
(A) said first polypeptide chain has the amino acid sequence of SEQ ID NO:59;
(B) said second polypeptide chain has the amino acid sequence of SEQ ID NO:60; and
(C) said third polypeptide chain has the amino acid sequence of SEQ ID NO:57.

8. A pharmaceutical composition comprising the B7-H3×CD3 bispecific monovalent Fc diabody of claim 7 and a physiologically acceptable carrier.

9. Use of the B7-H3×CD3 bispecific monovalent Fc diabody of claim 7 or the pharmaceutical composition of claim 8 in the treatment of a disease or condition associated with or characterized by the expression of B7-H3.

10. The use of claim 9, wherein said disease or condition associated with or characterized by the expression of B7-H3 is cancer.

11. The use of claim 10, wherein said cancer is selected from the group consisting of: an acute myeloid leukemia, an adrenal gland tumor, an AIDS-associated cancer, an alveolar soft part sarcoma, an astrocytic tumor, bladder cancer, bone cancer, a brain and spinal cord cancer, a metastatic brain tumor, a breast cancer, a carotid body tumors, a cervical cancer, a chondrosarcoma, a chordoma, a chromophobe renal cell carcinoma, a clear cell carcinoma, a colon cancer, a colorectal cancer, a cutaneous benign fibrous histiocytoma, a desmoplastic small round cell tumor, an ependymoma, a Ewing's tumor, an extraskeletal myxoid chondrosarcoma, a fibrogenesis imperfecta ossium, a fibrous dysplasia of the bone, a gallbladder or bile duct cancer, gastric cancer, a gestational trophoblastic disease, a germ cell tumor, a head and neck cancer, hepatocellular carcinoma, a glioblastoma, an islet cell tumor, a Kaposi's Sarcoma, a kidney cancer, a leukemia, a lipoma/benign lipomatous tumor, a liposarcoma/malignant lipomatous tumor, a liver cancer, a lymphoma, a lung cancer, a medulloblastoma, a melanoma, a meningioma, a malignant mesothelioma, a multiple endocrine neoplasia, a multiple myeloma, a myelodysplastic syndrome, a neuroblastoma, a neuroendocrine tumors, a non-small cell lung cancer, an ovarian cancer, a pancreatic cancer, a pharyngeal cancer, a papillary thyroid carcinoma, a parathyroid tumor, a pediatric cancer, a peripheral nerve sheath tumor, a phaeochromocytoma, a pituitary tumor, a prostate cancer, a posterious uveal melanoma, a rare hematologic disorder, a renal cell carcinoma, a renal metastatic cancer, a rhabdoid tumor, a rhabdomysarcoma, a sarcoma, a skin cancer, a soft-tissue sarcoma, a squamous cell cancer, a stomach cancer, a synovial sarcoma, a testicular cancer, a thymic carcinoma, a thymoma, a thyroid metastatic cancer, and a uterine cancer.

12. The use of claim 11, wherein said cancer is selected from the group consisting: bladder cancer, breast cancer, colorectal cancer, gastric cancer, glioblastoma, kidney cancer, lung cancer, melanoma, neuroblastoma, ovarian cancer, pancreatic cancer, pharyngeal cancer, prostate cancer, renal cell carcinoma, rhabdomyosarcoma, and squamous cell cancer of the head and neck (SCCHN).

13. A B7-H3×CD3 bispecific monovalent Fc diabody capable of specific binding to B7-H3 and to CD3, wherein the diabody comprises a first, a second and a third polypeptide chain, wherein said polypeptide chains form a covalently bonded complex, and wherein:
(A) said first polypeptide chain has the amino acid sequence of SEQ ID NO:61;
(B) said second polypeptide chain has the amino acid sequence of SEQ ID NO:62; and
(C) said third polypeptide chain has the amino acid sequence of SEQ ID NO:57.

14. A pharmaceutical composition comprising the B7-H3×CD3 bispecific monovalent Fc diabody of claim 15 and a physiologically acceptable carrier.

15. Use of the B7-H3×CD3 bispecific monovalent Fc diabody of claim 13 or the pharmaceutical composition of claim 14 in the treatment of a disease or condition associated with or characterized by the expression of B7-H3.

16. The use of claim 15, wherein said disease or condition associated with or characterized by the expression of B7-H3 is cancer.

17. The use of claim 16, wherein said cancer is selected from the group consisting of: an acute myeloid leukemia, an adrenal gland tumor, an AIDS-associated cancer, an alveolar soft part sarcoma, an astrocytic tumor, bladder cancer, bone cancer, a brain and spinal cord cancer, a metastatic brain tumor, a breast cancer, a carotid body tumors, a cervical cancer, a chondrosarcoma, a chordoma, a chromophobe renal cell carcinoma, a clear cell carcinoma, a colon cancer, a colorectal cancer, a cutaneous benign fibrous histiocytoma, a desmoplastic small round cell tumor, an ependymoma, a Ewing's tumor, an extraskeletal myxoid chondrosarcoma, a fibrogenesis imperfecta ossium, a fibrous dysplasia of the bone, a gallbladder or bile duct cancer, gastric cancer, a gestational trophoblastic disease, a germ cell tumor, a head and neck cancer, hepatocellular carcinoma, a glioblastoma, an islet cell tumor, a Kaposi's Sarcoma, a kidney cancer, a leukemia, a lipoma/benign lipomatous tumor, a liposarcoma/malignant lipomatous tumor, a liver cancer, a lymphoma, a lung cancer, a medulloblastoma, a melanoma, a meningioma, a malignant mesothelioma, a multiple endocrine neoplasia, a multiple myeloma, a myelodysplastic syndrome, a neuroblastoma, a neuroendocrine tumors, a non-small cell lung cancer, an ovarian cancer, a pancreatic cancer, a pharyngeal cancer, a papillary thyroid carcinoma, a parathyroid tumor, a pediatric cancer, a peripheral nerve sheath tumor, a phaeochromocytoma, a pituitary tumor, a prostate cancer, a posterious uveal melanoma, a rare hematologic disorder, a renal cell carcinoma, a renal metastatic cancer, a rhabdoid tumor, a rhabdomysarcoma, a sarcoma, a skin cancer, a soft-tissue sarcoma, a squamous cell cancer, a stomach cancer, a synovial sarcoma, a testicular cancer, a thymic carcinoma, a thymoma, a thyroid metastatic cancer, and a uterine cancer.

18. The use of claim 17, wherein said cancer is selected from the group consisting: bladder cancer, breast cancer, colorectal cancer, gastric cancer, glioblastoma, kidney cancer, lung cancer, melanoma, neuroblastoma, ovarian cancer, pancreatic cancer, pharyngeal cancer, prostate cancer, renal cell carcinoma, rhabdomyosarcoma, and squamous cell cancer of the head and neck (SCCHN).

19. A B7-H3×CD3 bispecific monovalent Fc diabody capable of specific binding to B7-H3 and to CD3, wherein the diabody comprises a first, a second and a third polypeptide chain, wherein said polypeptide chains form a covalently bonded complex, and wherein:
(A) said first polypeptide chain has the amino acid sequence of SEQ ID NO:63;
(B) said second polypeptide chain has the amino acid sequence of SEQ ID NO:64; and
(C) said third polypeptide chain has the amino acid sequence of SEQ ID NO:57.

20. A pharmaceutical composition comprising the B7-H3×CD3 bispecific monovalent Fc diabody of claim 19 and a physiologically acceptable carrier.

21. Use of the B7-H3×CD3 bispecific monovalent Fc diabody of claim 19 or the pharmaceutical composition of claim 20 in the treatment of a disease or condition associated with or characterized by the expression of B7-H3.

22. The use of claim 21, wherein said disease or condition associated with or characterized by the expression of B7-H3 is cancer.

23. The use of claim 22, wherein said cancer is selected from the group consisting of: an acute myeloid leukemia, an adrenal gland tumor, an AIDS-associated cancer, an alveolar soft part sarcoma, an astrocytic tumor, bladder cancer, bone cancer, a brain and spinal cord cancer, a metastatic brain tumor, a breast cancer, a carotid body tumors, a cervical cancer, a chondrosarcoma, a chordoma, a chromophobe renal cell carcinoma, a clear cell carcinoma, a colon cancer, a colorectal cancer, a cutaneous benign fibrous histiocytoma, a desmoplastic small round cell tumor, an ependymoma, a Ewing's tumor, an extraskeletal myxoid chondrosarcoma, a fibrogenesis imperfecta ossium, a fibrous dysplasia of the bone, a gallbladder or bile duct cancer, gastric cancer, a gestational trophoblastic disease, a germ cell tumor, a head and neck cancer, hepatocellular carcinoma, a glioblastoma, an islet cell tumor, a Kaposi's Sarcoma, a kidney cancer, a leukemia, a lipoma/benign lipomatous tumor, a liposarcoma/malignant lipomatous tumor, a liver cancer, a lymphoma, a lung cancer, a medulloblastoma, a melanoma, a meningioma, a malignant mesothelioma, a multiple endocrine neoplasia, a multiple myeloma, a myelodysplastic syndrome, a neuroblastoma, a neuroendocrine tumors, a non-small cell lung cancer, an ovarian cancer, a pancreatic cancer, a pharyngeal cancer, a papillary thyroid carcinoma, a parathyroid tumor, a pediatric cancer, a peripheral nerve sheath tumor, a phaeochromocytoma, a pituitary tumor, a prostate cancer, a posterious uveal melanoma, a rare hematologic disorder, a renal cell carcinoma, a renal metastatic cancer, a rhabdoid tumor, a rhabdomysarcoma, a sarcoma, a skin cancer, a soft-tissue sarcoma, a squamous cell cancer, a stomach cancer, a synovial sarcoma, a testicular cancer, a thymic carcinoma, a thymoma, a thyroid metastatic cancer, and a uterine cancer.

24. The use of claim 23, wherein said cancer is selected from the group consisting: bladder cancer, breast cancer, colorectal cancer, gastric cancer, glioblastoma, kidney cancer, lung cancer, melanoma, neuroblastoma, ovarian cancer, pancreatic cancer, pharyngeal cancer, prostate cancer, renal cell carcinoma, rhabdomyosarcoma, and squamous cell cancer of the head and neck (SCCHN).


SEQUENCE LISTING (68 sequences)

SEQ ID NO: 1 - protein, 8 aa, Artificial Sequence
  Linker 1
GGGSGGGG

SEQ ID NO: 2 - protein, 6 aa, Artificial Sequence
  Linker 2
GGCGGG

SEQ ID NO: 3 - protein, 5 aa, Artificial Sequence
  Alternative Linker 2
ASTKG

SEQ ID NO: 4 - protein, 10 aa, Artificial Sequence
  Linker 3
DKTHTCPPCP

SEQ ID NO: 5 - protein, 13 aa, Artificial Sequence
  Spacer-Linker 3
GGGDKTHTCPPCP

SEQ ID NO: 6 - protein, 7 aa, Artificial Sequence
  Heterodimer-Promoting Domain
GVEPKSC

SEQ ID NO: 7 - protein, 6 aa, Artificial Sequence
  Heterodimer-Promoting Domain
VEPKSC

SEQ ID NO: 8 - protein, 7 aa, Artificial Sequence
  Heterodimer-Promoting Domain
GFNRGEC

SEQ ID NO: 9 - protein, 6 aa, Artificial Sequence
  Heterodimer-Promoting Domain
FNRGEC

SEQ ID NO: 10 - protein, 28 aa, Artificial Sequence
  “E-coil” Heterodimer-Promoting Domain
EVAALEKEVAALEKEVAALEKEVAALEK

SEQ ID NO: 11 - protein, 28 aa, Artificial Sequence
  “K-coil” Heterodimer-Promoting Domain
KVAALKEKVAALKEKVAALKEKVAALKE

SEQ ID NO: 12 - protein, 28 aa, Artificial Sequence
  Cysteine-Containing “E-coil” Heterodimer-Promoting Domain
EVAACEKEVAALEKEVAALEKEVAALEK

SEQ ID NO: 13 - protein, 28 aa, Artificial Sequence
  Cysteine-Containing “K-coil” Heterodimer- Promoting Domain
KVAACKEKVAALKEKVAALKEKVAALKE

SEQ ID NO: 14 - protein, 217 aa, Homo sapiens
  MISC_FEATURE (1)..(217): Human IgG1 CH2-CH3 Domain
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 217 aa, Artificial Sequence
  “Knob-Bearing” Human IgG1 CH2-CH3 Domain
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 217 aa, Artificial Sequence
  “Hole-Bearing” Human IgG1 CH2-CH3 Domain
APEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRYTQKSLSLSPGK

SEQ ID NO: 17 - protein, 107 aa, Mus musculus
  MISC_FEATURE (1)..(107): Light Chain Variable Domain of B7-H3 mAb A
DIQLTQSPSFLSASVGDRVTITCKASQNVDTNVAWYQQKPGKAPKALIYSASYRYSGVPSRFSGSGSGTDFTLTISSLQPEDFATYYCQQYNNYPFTFGQGTKLEIK

SEQ ID NO: 18 - protein, 11 aa, Mus musculus
  MISC_FEATURE (1)..(11): Light Chain CDR1 of B7-H3 mAb A
KASQNVDTNVA

SEQ ID NO: 19 - protein, 7 aa, Mus musculus
  MISC_FEATURE (1)..(7): Light Chain CDR2 of B7-H3 mAb A
SASYRYS

SEQ ID NO: 20 - protein, 9 aa, Mus musculus
  MISC_FEATURE (1)..(9): Light Chain CDR3 of B7-H3 mAb A
QQYNNYPFT

SEQ ID NO: 21 - protein, 122 aa, Mus musculus
  MISC_FEATURE (1)..(122): Heavy Chain Variable Domain of B7-H3 mAb A
EVQLVESGGGLVQPGGSLRLSCAASGFTFSSFGMHWVRQAPGKGLEWVAYISSDSSAIYYADTVKGRFTISRDNAKNSLYLQMNSLRDEDTAVYYCGRGRENIYYGSRLDYWGQGTTVTVSS

SEQ ID NO: 22 - protein, 5 aa, Mus musculus
  MISC_FEATURE (1)..(5): Heavy Chain CDR1 of B7-H3 mAb A
SFGMH

SEQ ID NO: 23 - protein, 17 aa, Mus musculus
  MISC_FEATURE (1)..(17): Heavy Chain CDR2 of B7-H3 mAb A
YISSDSSAIYYADTVKG

SEQ ID NO: 24 - protein, 13 aa, Mus musculus
  MISC_FEATURE (1)..(13): Heavy Chain CDR3 of B7-H3 mAb A
GRENIYYGSRLDY

SEQ ID NO: 25 - protein, 107 aa, Mus musculus
  MISC_FEATURE (1)..(107): Light Chain Variable Domain of B7-H3 mAb B
DIQMTQSPSSLSASVGDRVTITCRASQDISNYLNWYQQKPGKAPKLLIYYTSRLHSGVPSRFSGSGSGTDFTLTISSLQPEDIATYYCQQGNTLPPTFGGGTKLEIK

SEQ ID NO: 26 - protein, 11 aa, Mus musculus
  MISC_FEATURE (1)..(11): Light Chain CDR1 of B7-H3 mAb B
RASQDISNYLN

SEQ ID NO: 27 - protein, 7 aa, Mus musculus
  MISC_FEATURE (1)..(7): Light Chain CDR2 of B7-H3 mAb B
YTSRLHS

SEQ ID NO: 28 - protein, 9 aa, Mus musculus
  MISC_FEATURE (1)..(9): Light Chain CDR3 of B7-H3 mAb B
QQGNTLPPT

SEQ ID NO: 29 - protein, 120 aa, Mus musculus
  MISC_FEATURE (1)..(120): Heavy Chain Variable Domain of B7-H3 mAb B
QVQLVQSGAEVKKPGASVKVSCKASGYTFTSYWMQWVRQAPGQGLEWMGTIYPGDGDTRYTQKFKGRVTITADKSTSTAYMELSSLRSEDTAVYYCARRGIPRLWYFDVWGQGTTVTVSS

SEQ ID NO: 30 - protein, 5 aa, Mus musculus
  MISC_FEATURE (1)..(5): Heavy Chain CDR1 of B7-H3 mAb B
SYWMQ

SEQ ID NO: 31 - protein, 17 aa, Mus musculus
  MISC_FEATURE (1)..(17): Heavy Chain CDR2 of B7-H3 mAb B
TIYPGDGDTRYTQKFKG

SEQ ID NO: 32 - protein, 11 aa, Mus musculus
  MISC_FEATURE (1)..(11): Heavy Chain CDR3 of B7-H3 mAb B
RGIPRLWYFDV

SEQ ID NO: 33 - protein, 107 aa, Mus musculus
  MISC_FEATURE (1)..(107): Light Chain Variable Domain of B7-H3 mAb C
DIQMTQSPSSLSASVGDRVTITCRASQSISSYLNWYQQKPGKAPKLLIYYTSRLQSGVPSRFSGSGSGTDFTLTISSLQPEDIATYYCQQGNTLPPTFGGGTKLEIK

SEQ ID NO: 34 - protein, 11 aa, Mus musculus
  MISC_FEATURE (1)..(11): Light Chain CDR1 of B7-H3 mAb C
RASQSISSYLN

SEQ ID NO: 35 - protein, 7 aa, Mus musculus
  MISC_FEATURE (1)..(7): Light Chain CDR2 of B7-H3 mAb C
YTSRLQS

SEQ ID NO: 36 - protein, 9 aa, Mus musculus
  MISC_FEATURE (1)..(9): Light Chain CDR3 of B7-H3 mAb C
QQGNTLPPT

SEQ ID NO: 37 - protein, 120 aa, Mus musculus
  MISC_FEATURE (1)..(120): Heavy Chain Variable Domain of B7-H3 mAb C
QVQLVQSGAEVKKPGASVKVSCKASGYTFTSYWMQWVRQAPGQGLEWMGTIYPGGGDTRYTQKFQGRVTITADKSTSTAYMELSSLRSEDTAVYYCARRGIPRLWYFDVWGQGTTVTVSS

SEQ ID NO: 38 - protein, 5 aa, Mus musculus
  MISC_FEATURE (1)..(5): Heavy Chain CDR1 of B7-H3 mAb C
SYWMQ

SEQ ID NO: 39 - protein, 17 aa, Mus musculus
  MISC_FEATURE (1)..(17): Heavy Chain CDR2 of B7-H3 mAb C
TIYPGGGDTRYTQKFQG

SEQ ID NO: 40 - protein, 11 aa, Mus musculus
  MISC_FEATURE (1)..(11): Heavy Chain CDR3 of B7-H3 mAb C
RGIPRLWYFDV

SEQ ID NO: 41 - protein, 110 aa, Mus musculus
  MISC_FEATURE (1)..(110): Light Chain Variable Domain of CD3 mAb A
QAVVTQEPSLTVSPGGTVTLTCRSSTGAVTTSNYANWVQQKPGQAPRGLIGGTNKRAPWTPARFSGSLLGGKAALTITGAQAEDEADYYCALWYSNLWVFGGGTKLTVLG

SEQ ID NO: 42 - protein, 14 aa, Mus musculus
  MISC_FEATURE (1)..(14): Light Chain CDR1 of CD3 mAb A
RSSTGAVTTSNYAN

SEQ ID NO: 43 - protein, 7 aa, Mus musculus
  MISC_FEATURE (1)..(7): Light Chain CDR2 of CD3 mAb A
GTNKRAP

SEQ ID NO: 44 - protein, 9 aa, Mus musculus
  MISC_FEATURE (1)..(9): Light Chain CDR3 of CD3 mAb A
ALWYSNLWV

SEQ ID NO: 45 - protein, 125 aa, Mus musculus
  MISC_FEATURE (1)..(125): Heavy Chain Variable Domain of CD3 mAb A
EVQLVESGGGLVQPGGSLRLSCAASGFTFSTYAMNWVRQAPGKGLEWVGRIRSKYNNYATYYADSVKDRFTISRDDSKNSLYLQMNSLKTEDTAVYYCVRHGNFGNSYVSWFAYWGQGTLVTVSS

SEQ ID NO: 46 - protein, 5 aa, Mus musculus
  MISC_FEATURE (1)..(5): Heavy Chain CDR1 of CD3 mAb A
TYAMN

SEQ ID NO: 47 - protein, 19 aa, Mus musculus
  MISC_FEATURE (1)..(19): Heavy Chain CDR2 of CD3 mAb A
RIRSKYNNYATYYADSVKD

SEQ ID NO: 48 - protein, 14 aa, Mus musculus
  MISC_FEATURE (1)..(14): Heavy Chain CDR3 of CD3 mAb A
HGNFGNSYVSWFAY

SEQ ID NO: 49 - protein, 19 aa, Artificial Sequence
  Heavy Chain CDR3 of CD3 mAb A Containing Aspartate to Glycine Substitution at Kabat Position 65
RIRSKYNNYATYYADSVKG

SEQ ID NO: 50 - protein, 125 aa, Artificial Sequence
  Heavy Chain Variable Domain of CD3 mAb A Containing Aspartate to Glycine Substitution at Kabat Position 65
EVQLVESGGGLVQPGGSLRLSCAASGFTFSTYAMNWVRQAPGKGLEWVGRIRSKYNNYATYYADSVKGRFTISRDDSKNSLYLQMNSLKTEDTAVYYCVRHGNFGNSYVSWFAYWGQGTLVTVSS

SEQ ID NO: 51 - protein, 5 aa, Artificial Sequence
  Linker 4
APSSS

SEQ ID NO: 52 - protein, 8 aa, Artificial Sequence
  Alternative Linker 4
APSSSPME

SEQ ID NO: 53 - protein, 504 aa, Artificial Sequence
  First Polypeptide Chain of B7-H3 x CD3 Bispecific DART-A Diabody
DIQLTQSPSFLSASVGDRVTITCKASQNVDTNVAWYQQKPGKAPKALIYSASYRYSGVPSRFSGSGSGTDFTLTISSLQPEDFATYYCQQYNNYPFTFGQGTKLEIKGGGSGGGGEVQLVESGGGLVQPGGSLRLSCAASGFTFSTYAMNWVRQAPGKGLEWVGRIRSKYNNYATYYADSVKDRFTISRDDSKNSLYLQMNSLKTEDTAVYYCVRHGNFGNSYVSWFAYWGQGTLVTVSSGGCGGGEVAALEKEVAALEKEVAALEKEVAALEKGGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 54 - DNA, 1512 bp, Artificial Sequence
  Polynucleotide Encoding the First Polypeptide Chain of B7-H3 x CD3 Bispecific DART-A Diabody
gacatccagctgacccagtccccctccttcctgtctgcctccgtgggcgacagagtgaccatcacatgcaaggcctcccagaacgtggacaccaacgtggcctggtatcagcagaagcctggcaaggcccctaaggcgctgatctactccgcctcctaccggtactccggcgtgccttccaggttctccggctccggctctggcaccgacttcaccctgaccatctccagcctgcagcctgaggacttcgccacctactactgccagcagtacaacaactaccctttcaccttcggccagggcaccaagctggaaatcaagggaggcggatccggcggcggaggcgaggtgcagctggtggagtctgggggaggcttggtccagcctggagggtccctgagactctcctgtgcagcctctggattcaccttcagcacatacgctatgaattgggtccgccaggctccagggaaggggctggagtgggttggaaggatcaggtccaagtacaacaattatgcaacctactatgccgactctgtgaaggatagattcaccatctcaagagatgattcaaagaactcactgtatctgcaaatgaacagcctgaaaaccgaggacacggccgtgtattactgtgtgagacacggtaacttcggcaattcttacgtgtcttggtttgcttattggggacaggggacactggtgactgtgtcttccggaggatgtggcggtggagaagtggccgcactggagaaagaggttgctgctttggagaaggaggtcgctgcacttgaaaaggaggtcgcagccctggagaaaggcggcggggacaaaactcacacatgcccaccgtgcccagcacctgaagccgcggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgtggtgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 55 - protein, 274 aa, Artificial Sequence
  Second Polypeptide Chain of B7-H3 x CD3 Bispecific DART-A Diabody
QAVVTQEPSLTVSPGGTVTLTCRSSTGAVTTSNYANWVQQKPGQAPRGLIGGTNKRAPWTPARFSGSLLGGKAALTITGAQAEDEADYYCALWYSNLWVFGGGTKLTVLGGGGSGGGGEVQLVESGGGLVQPGGSLRLSCAASGFTFSSFGMHWVRQAPGKGLEWVAYISSDSSAIYYADTVKGRFTISRDNAKNSLYLQMNSLRDEDTAVYYCGRGRENIYYGSRLDYWGQGTTVTVSSGGCGGGKVAALKEKVAALKEKVAALKEKVAALKE

SEQ ID NO: 56 - DNA, 822 bp, Artificial Sequence
  Polynucleotide Encoding the Second Polypeptide Chain of B7-H3 x CD3 Bispecific DART-A Diabody
caggctgtggtgactcaggagccttcactgaccgtgtccccaggcggaactgtgaccctgacatgcagatccagcacaggcgcagtgaccacatctaactacgccaattgggtgcagcagaagccaggacaggcaccaaggggcctgatcgggggtacaaacaaaagggctccctggacccctgcacggttttctggaagtctgctgggcggaaaggccgctctgactattaccggggcacaggccgaggacgaagccgattactattgtgctctgtggtatagcaatctgtgggtgttcgggggtggcacaaaactgactgtgctgggagggggtggatccggcggcggaggcgaggtgcagctggtcgagtctggcggaggactggtgcagcctggcggctccctgagactgtcttgcgccgcctccggcttcaccttctccagcttcggcatgcactgggtccgccaggctccaggcaagggactggaatgggtggcctacatctcctccgactcctccgccatctactacgccgacaccgtgaagggcaggttcaccatctcccgggacaacgccaagaactccctgtacctgcagatgaactccctgcgggacgaggacaccgccgtgtactactgcggcagaggccgggagaatatctactacggctcccggctggattattggggccagggcaccaccgtgaccgtgtcctccggaggatgtggcggtggaaaagtggccgcactgaaggagaaagttgctgctttgaaagagaaggtcgccgcacttaaggaaaaggtcgcagccctgaaagag

SEQ ID NO: 57 - protein, 227 aa, Artificial Sequence
  Third Polypeptide Chain of B7-H3 x CD3 Bispecific DART-A Diabody
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLSCAVKGFYPSDIAVEWESNGQPENNYKTTPPVLDSDGSFFLVSKLTVDKSRWQQGNVFSCSVMHEALHNRYTQKSLSLSPGK

SEQ ID NO: 58 - DNA, 681 bp, Artificial Sequence
  Polynucleotide Encoding the Third Polypeptide Chain of B7-H3 x CD3 Bispecific DART-A Diabody
gacaaaactcacacatgcccaccgtgcccagcacctgaagccgcggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgagttgcgcagtcaaaggcttctatcccagcgacatcgccgtggagtgggagagcaatgggcagccggagaacaactacaagaccacgcctcccgtgctggactccgacggctccttcttcctcgtcagcaagctcaccgtggacaagagcaggtggcagcaggggaacgtcttctcatgctccgtgatgcatgaggctctgcacaaccgctacacgcagaagagcctctccctgtctccgggtaaa

SEQ ID NO: 59 - protein, 504 aa, Artificial Sequence
  First Polypeptide Chain of B7-H3 x CD3 Bispecific DART-B Diabody
DIQMTQSPSSLSASVGDRVTITCRASQDISNYLNWYQQKPGKAPKLLIYYTSRLHSGVPSRFSGSGSGTDFTLTISSLQPEDIATYYCQQGNTLPPTFGGGTKLEIKGGGSGGGGEVQLVESGGGLVQPGGSLRLSCAASGFTFSTYAMNWVRQAPGKGLEWVGRIRSKYNNYATYYADSVKDRFTISRDDSKNSLYLQMNSLKTEDTAVYYCVRHGNFGNSYVSWFAYWGQGTLVTVSSGGCGGGEVAALEKEVAALEKEVAALEKEVAALEKGGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 60 - protein, 272 aa, Artificial Sequence
  Second Polypeptide Chain of B7-H3 x CD3 Bispecific DART-B Diabody
QAVVTQEPSLTVSPGGTVTLTCRSSTGAVTTSNYANWVQQKPGQAPRGLIGGTNKRAPWTPARFSGSLLGGKAALTITGAQAEDEADYYCALWYSNLWVFGGGTKLTVLGGGGSGGGGQVQLVQSGAEVKKPGASVKVSCKASGYTFTSYWMQWVRQAPGQGLEWMGTIYPGDGDTRYTQKFKGRVTITADKSTSTAYMELSSLRSEDTAVYYCARRGIPRLWYFDVWGQGTTVTVSSGGCGGGKVAALKEKVAALKEKVAALKEKVAALKE

SEQ ID NO: 61 - protein, 504 aa, Artificial Sequence
  First Polypeptide Chain of B7-H3 x CD3 Bispecific DART-C Diabody
DIQMTQSPSSLSASVGDRVTITCRASQSISSYLNWYQQKPGKAPKLLIYYTSRLQSGVPSRFSGSGSGTDFTLTISSLQPEDIATYYCQQGNTLPPTFGGGTKLEIKGGGSGGGGEVQLVESGGGLVQPGGSLRLSCAASGFTFSTYAMNWVRQAPGKGLEWVGRIRSKYNNYATYYADSVKGRFTISRDDSKNSLYLQMNSLKTEDTAVYYCVRHGNFGNSYVSWFAYWGQGTLVTVSSGGCGGGEVAALEKEVAALEKEVAALEKEVAALEKGGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 62 - protein, 272 aa, Artificial Sequence
  Second Polypeptide Chain of B7-H3 x CD3 Bispecific DART-C Diabody
QAVVTQEPSLTVSPGGTVTLTCRSSTGAVTTSNYANWVQQKPGQAPRGLIGGTNKRAPWTPARFSGSLLGGKAALTITGAQAEDEADYYCALWYSNLWVFGGGTKLTVLGGGGSGGGGQVQLVQSGAEVKKPGASVKVSCKASGYTFTSYWMQWVRQAPGQGLEWMGTIYPGGGDTRYTQKFQGRVTITADKSTSTAYMELSSLRSEDTAVYYCARRGIPRLWYFDVWGQGTTVTVSSGGCGGGKVAALKEKVAALKEKVAALKEKVAALKE

SEQ ID NO: 63 - protein, 503 aa, Artificial Sequence
  First Polypeptide Chain of B7-H3 x CD3 Bispecific DART-D Diabody
DIQMTQSPSSLSASVGDRVTITCRASQSISSYLNWYQQKPGKAPKLLIYYTSRLQSGVPSRFSGSGSGTDFTLTISSLQPEDIATYYCQQGNTLPPTFGGGTKLEIKGGGSGGGGEVQLVESGGGLVQPGGSLRLSCAASGFTFSTYAMNWVRQAPGKGLEWVGRIRSKYNNYATYYADSVKGRFTISRDDSKNSLYLQMNSLKTEDTAVYYCVRHGNFGNSYVSWFAYWGQGTLVTVSSASTKGEVAACEKEVAALEKEVAALEKEVAALEKGGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 64 - protein, 271 aa, Artificial Sequence
  Second Polypeptide Chain of B7-H3 x CD3 Bispecific DART-D Diabody
QAVVTQEPSLTVSPGGTVTLTCRSSTGAVTTSNYANWVQQKPGQAPRGLIGGTNKRAPWTPARFSGSLLGGKAALTITGAQAEDEADYYCALWYSNLWVFGGGTKLTVLGGGGSGGGGQVQLVQSGAEVKKPGASVKVSCKASGYTFTSYWMQWVRQAPGQGLEWMGTIYPGGGDTRYTQKFQGRVTITADKSTSTAYMELSSLRSEDTAVYYCARRGIPRLWYFDVWGQGTTVTVSSASTKGKVAACKEKVAALKEKVAALKEKVAALKE

SEQ ID NO: 65 - protein, 112 aa, Mus musculus
  MISC_FEATURE (1)..(112): Light Chain Varible Domain of Anti-Fluorescein Antibody 4-4-20
DVVMTQTPFSLPVSLGDQASISCRSSQSLVHSNGNTYLRWYLQKPGQSPKVLIYKVSNRFSGVPDRFSGSGSGTDFTLKISRVEAEDLGVYFCSQSTHVPWTFGGGTKLEIK

SEQ ID NO: 66 - protein, 118 aa, Mus musculus
  MISC_FEATURE (1)..(118): Heavy Chain Varible Domain of Anti-Fluorescein Antibody 4-4-20
EVKLDETGGGLVQPGRPMKLSCVASGFTFSDYWMNWVRQSPEKGLEWVAQIRNKPYNYETYYSDSVKGRFTISRDDSKSSVYLQMNNLRVEDMGIYYCTGSYYGMDYWGQGTSVTVSS

SEQ ID NO: 67 - protein, 509 aa, Artificial Sequence
  First Polypeptide of Anti-Fluorescein Diabody (Control DART)
DVVMTQTPFSLPVSLGDQASISCRSSQSLVHSNGNTYLRWYLQKPGQSPKVLIYKVSNRFSGVPDRFSGSGSGTDFTLKISRVEAEDLGVYFCSQSTHVPWTFGGGTKLEIKGGGSGGGGEVQLVESGGGLVQPGGSLRLSCAASGFTFSTYAMNWVRQAPGKGLEWVGRIRSKYNNYATYYADSVKGRFTISRDDSKNSLYLQMNSLKTEDTAVYYCVRHGNFGNSYVSWFAYWGQGTLVTVSSGGCGGGEVAALEKEVAALEKEVAALEKEVAALEKGGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 68 - protein, 270 aa, Artificial Sequence
  Second Polypeptide of Anti-Fluorescein Diabody (Control DART)
QAVVTQEPSLTVSPGGTVTLTCRSSTGAVTTSNYANWVQQKPGQAPRGLIGGTNKRAPWTPARFSGSLLGGKAALTITGAQAEDEADYYCALWYSNLWVFGGGTKLTVLGGGGSGGGGEVKLDETGGGLVQPGRPMKLSCVASGFTFSDYWMNWVRQSPEKGLEWVAQIRNKPYNYETYYSDSVKGRFTISRDDSKSSVYLQMNNLRVEDMGIYYCTGSYYGMDYWGQGTSVTVSSGGCGGGKVAALKEKVAALKEKVAALKEKVAALKE